(19) United States
(12) Patent Application Publication
(10) Pub. No.: US 2018/0271837 A1
(43) Pub. Date: Sep. 27, 2018

(54) ISOXAZOLE CARBOXAMIDE COMPOUNDS AND USES THEREOF

(71) Applicant: NOVARTIS AG, Basel (CH)

(72) Inventors: Rohan Eric John BECKWITH, Cambridge, MA (US); Hua JIANG, Shanghai (CN); Ce WANG, Shanghai (CN)

(73) Assignee: NOVARTIS AG, Basel (CH)

(21) Appl. No.: 15/934,176

(22) Filed: Mar. 23, 2018

(30) Foreign Application Priority Data
Mar. 24, 2017 (CN) PCT/CN2017/078060

(51) Int. Cl. A61K 31/422; A61K 31/16; A61P 27/16

(52) U.S. Cl. CPC A61K 31/422; A61K 31/5377; A61P 27/16; A61K 31/16

(57) ABSTRACT

A compound of Formula (I)) or or a pharmaceutically acceptable salt thereof, is provided that has been shown to be useful for treating hearing loss or balance disorder:

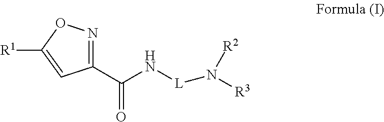

wherein R¹ through R³, and L are as defined herein.

### CLAIM OF PRIORITY

This application claims priority from PCT/CN2017/078060 filed Mar. 24, 2017, which is incorporated herein by reference in its entirety.

### FIELD OF THE INVENTION

The present disclosure relates to compounds, compositions comprising such compounds, and their use for the treatment of hearing loss or balance disorder.

### BACKGROUND OF THE INVENTION

Hair cells in the inner ear are essential for hearing and balance. If hair cells are damaged in any way, human beings would suffer hearing loss or balance disorder. The human inner ear contains only about 15,000 hair cells per cochlea at birth, and, although these cells can be lost as a result of various genetic or environmental factors, the lost or damaged cells cannot be replaced. However, overexpression of the transcription factor, Atoh1, can induce sensory hair cells from epithelial cells in the sensory organ of the cochlea and the organ of Corti (Zheng and Gao, Nat Neurosci 2000; 3:580-586; Kawamoto et al., J Neurosci 2003; 23:4395-4400; Izumikawa M et al., Nat Med. 2005; 11: 271-276; Gubbels et al., Nature 2008; 455:537-541). Therefore, there is a need to discover therapeutic compositions and methods that induce Atoh1 expression and promote mammalian hair cell regeneration.

### SUMMARY OF THE INVENTION

The present disclosure provides compounds, pharmaceutically acceptable salts thereof, pharmaceutical compositions thereof and combinations thereof, which are useful to treat hearing loss or balance disorder. The present disclosure further provides methods of treating hearing loss or balance disorder, comprising administering to a subject in need thereof an effective amount of a compound of the present disclosure, or a pharmaceutically acceptable salt thereof.

One aspect of the present disclosure provides a compound of Formula (I) or a pharmaceutically acceptable salt thereof:

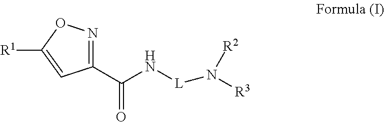

Another aspect of the present disclosure provides a pharmaceutical composition comprising a therapeutically effective amount of a compound of Formula (I) or a pharmaceutically acceptable salt thereof, or subformulae thereof, and one or more pharmaceutically acceptable carriers.

In yet another aspect of present disclosure, a pharmaceutical combination is provided which comprises a therapeutically effective amount of a compound of Formula (I) or a pharmaceutically acceptable salt thereof, or subformulae thereof, and one or more therapeutically active agents.

In yet another aspect of present disclosure, a method is provided for treating hearing loss or balance disorder, which comprises administering to a subject in need thereof a therapeutically effective amount of a compound of Formula (I) or a pharmaceutically acceptable salt thereof, or subformulae thereof.

In yet another aspect of the present disclosure, processes are provided for preparing compounds of Formula (I) or a pharmaceutically acceptable salt thereof, or subformulae thereof.

### DETAILED DESCRIPTION

Various (enumerated) embodiments of the disclosure are described herein. It will be recognized that features specified in each embodiment may be combined with other specified features to provide further embodiments of the present disclosure.

### Embodiment 1

A compound of Formula (I)

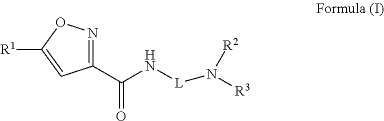

or a pharmaceutically acceptable salt thereof, wherein:

R¹ is selected from phenyl, thienyl, and furanyl, which are each independently optionally substituted by 1-2 F;

L is C₅-C₆ alkylene optionally substituted with 1-4 substituents independently selected from C₁₋₆ alkyl and halogen, wherein optionally a C₁₋₆ alkyl substituent is taken together with the carbon atoms to which it is attached to form a 3-membered cycloalkyl ring;

R² and R³ are taken together with the nitrogen atom to which they are attached form a 4- to 10-membered heterocyclyl comprising carbon atoms and 1-3 heteroatoms independently selected from N and O, which is optionally substituted with 1-4 R⁴;

each R⁴ is independently selected from C₁₋₆ alkyl, C₃₋₈ cycloalkyl, halogen, (C₀-C₃ alkylene)-CN, C₁₋₆ haloalkyl, C₁-C₆ haloalkoxy, (C₀-C₆ alkylene)-OR⁵, (═O), NH(C═O)R⁵, NH(C═O)OR⁷, NH(C═O)N(R⁵)₂, (C═O)N(R⁷)₂, (C═O)R⁵, (C═O)O(C₁₋₆alkyl), (C═O)O(C₃₋₈ cycloalkyl), S(═O)₂R⁵, S(═O)₂N(R⁷)₂, NHS(═O)₂R⁵, phenyl optionally substituted with 1-3 R⁶ and 5- to 6-membered heteroaryl comprising carbon atoms and 1-3 heteroatoms independently selected from N, O and S optionally substituted with 1-3 R⁶;

each R⁵ is independently selected from H, C₁₋₆ alkyl and C₃₋₈ cycloalkyl;

each R⁶ is independently selected from C₁₋₆ alkyl, C₃₋₈ cycloalkyl, halogen, CN, C₁₋₆ haloalkyl, C₁-C₆ haloalkoxy, OR⁵, N(R⁵)₂, NH(C═O)R⁵, (C═O)N(R⁵)₂, (C═O)R⁵, (C═O)OR⁵, S(═O)₂R⁵ and S(═O)₂N(R⁵)₂; and

each R⁷ is independently selected from H, C₁₋₆ alkyl, C₃₋₈ cycloalkyl optionally substituted with 1-2 OR⁵, (C₀-C₃ alkylene)-CN and (C₀-C₃ alkylene)-OR⁵.

### Embodiment 2

A compound or a pharmaceutically acceptable salt thereof according to Embodiment 1, wherein R¹ is selected from phenyl, phenyl substituted with one F, 2-thienyl, 3-thienyl, 2-furanyl and 3-furanyl.

### Embodiment 3

A compound or a pharmaceutically acceptable salt thereof according to Embodiment 1 or 2, wherein R¹ is

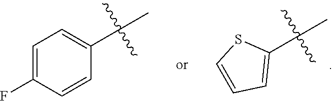 [SMILES: *c1ccc(F)cc1 ; *c1cccs1]

### Embodiment 4

A compound or a pharmaceutically acceptable salt thereof according to Embodiment 1, wherein L is C₅ alkylene optionally substituted with 1-4 halogen.

### Embodiment 5

A compound or a pharmaceutically acceptable salt thereof according to any one of the Embodiments 1-4, wherein L is C₅ alkylene optionally substituted with two F.

### Embodiment 6

A compound or a pharmaceutically acceptable salt thereof according to any one of the Embodiments 1-5, wherein R² and R³ are taken together with the nitrogen atom to which they are attached form a 4- to 10-membered heterocyclyl having the structure selected from:

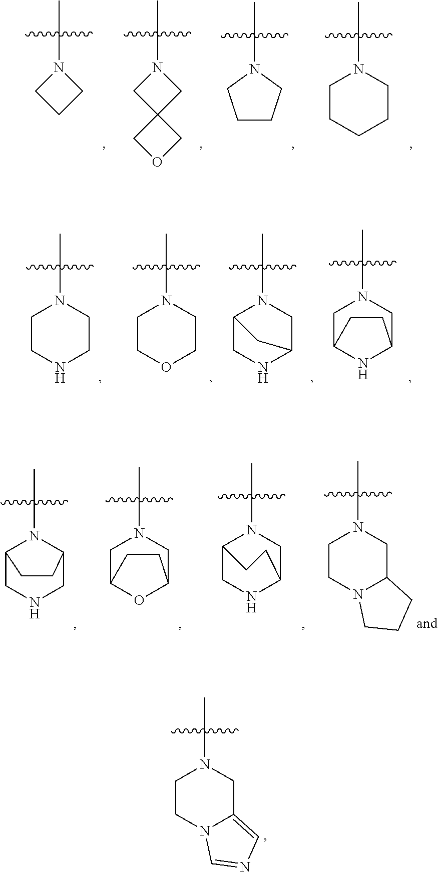

which are each independently optionally substituted with 1-2 R⁴.

### Embodiment 7

A compound or a pharmaceutically acceptable salt thereof according to any one of the Embodiments 1-6, wherein R² and R³ are taken together with the nitrogen atom to which they are attached form a 4- to 10-membered heterocyclyl having the structure selected from:

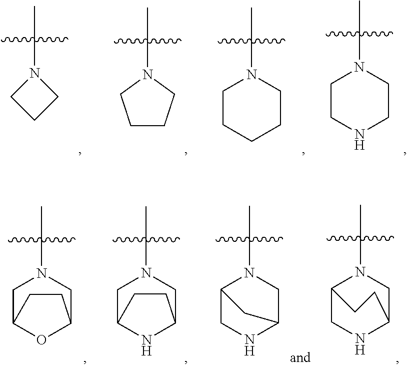

which are each independently optionally substituted with 1-2 R⁴.

### Embodiment 8

A compound or a pharmaceutically acceptable salt thereof according to any one of the Embodiments 1-7, wherein each R⁴ is independently selected C₁₋₆ alkyl, halogen, (C₀-C₃ alkylene)-CN, (C₀-C₆ alkylene)-OR⁵, (═O), NH(C═O)R⁵, NH(C═O)OR⁷, NH(C═O)N(R⁵)₂, (C═O)N(R⁷)₂, (C═O)R⁵, (C═O)O(C₁₋₆ alkyl), (C═O)O(C₃₋₈cycloalkyl), S(═O)₂N(R⁷)₂, NHS(═O)₂R⁵, phenyl optionally substituted with 1-3 R⁶ and 5- to 6-membered heteroaryl comprising carbon atoms and 1-3 heteroatoms independently selected from N, O and S optionally substituted with 1-3 R⁶.

### Embodiment 9

A compound or a pharmaceutically acceptable salt thereof according to any one of the Embodiments 1-8, wherein each R⁴ is independently selected from CH₃, CH₂CH(CH₃)₂, F, CN, CH₂—CN, OH, OCH₃, CH₂—OH, (CH₂)₂—OH, NH(C═O)OCH₃, NH(C═O)CH₃, NH(C═O)NHCH₃, (C═O)NH₂, (C═O)NHCH₃, (C═O)NH(cyclopentyl-OH), (C═O)NH(CH₂—CN), (C═O)NH(CH₂CH₂—CN), (C═O)NH(CH₂CH₂—OH), C(═O)CH₃, S(═O)₂NH₂, NHS(═O)₂CH₃, phenyl and imidazolyl.

### Embodiment 10

A compound or a pharmaceutically acceptable salt thereof according to any one of the Embodiments 1-9, wherein each R⁴ is independently selected from CH₃, F, (CH₂)₂—OH, (C═O)NH₂, S(═O)₂NH₂, (C═O)NH(CH₂—CN), (C═O)NH(CH₂CH₂—CN), (C═O)NH(cyclopentyl-OH) and NHS(═O)₂CH₃.

### Embodiment 11

A compound or a pharmaceutically acceptable salt thereof according to Embodiment 1 selected from:
Example 8: N-(5-(4-methylpiperazin-1-yl)pentyl)-5-(thiophen-2-yl)isoxazole-3-carboxamide;
Example 19: N-(5-(3-(methylsulfonamido)azetidin-1-yl)pentyl)-5-(thiophen-2-yl)isoxazole-3-carboxamide;
Example 27: N-(5-(3-carbamoylazetidin-1-yl)pentyl)-5-(thiophen-2-yl)isoxazole-3-carboxamide;
Example 45: (S)—N-(5-(3-fluoropyrrolidin-1-yl)pentyl)-5-(thiophen-2-yl)isoxazole-3-carboxamide;
Example 51: N-(5-(8-oxa-3-azabicyclo[3.2.1]octan-3-yl)pentyl)-5-(thiophen-2-yl)isoxazole-3-carboxamide;
Example 52: N-(5-(5-methyl-2,5-diazabicyclo[2.2.2]octan-2-yl)pentyl)-5-(thiophen-2-yl)isoxazole-3-carboxamide;
Example 54: N-(5-(4-(2-hydroxyethyl)piperazin-1-yl)pentyl)-5-(thiophen-2-yl)isoxazole-3-carboxamide;
Example 61: N-(5-(3-(methylcarbamoyl)azetidin-1-yl)pentyl)-5-(thiophen-2-yl)isoxazole-3-carboxamide;
Example 65: N-(5-(3-carbamoylazetidin-1-yl)pentyl)-5-(4-fluorophenyl)isoxazole-3-carboxamide;
Example 72: N-(5-(3-sulfamoylazetidin-1-yl)pentyl)-5-(thiophen-2-yl)isoxazole-3-carboxamide;
Example 73: 5-(5-fluorothiophen-2-yl)-N-(5-(4-methylpiperazin-1-yl)pentyl)isoxazole-3-carboxamide;
Example 74: N-(3,3-difluoro-5-(4-methylpiperazin-1-yl)pentyl)-5-(thiophen-2-yl)isoxazole-3-carboxamide;
Example 77: N-(5-(3-((cyanomethyl)carbamoyl)azetidin-1-yl)pentyl)-5-(thiophen-2-yl)isoxazole-3-carboxamide;
Example 83: N-(5-(3-((2-hydroxycyclopentyl)carbamoyl)azetidin-1-yl)pentyl)-5-(thiophen-2-yl) isoxazole-3-carboxamide;
Example 85: 5-(4-fluorophenyl)-N-(5-(3-(methylcarbamoyl)azetidin-1-yl)pentyl)isoxazole-3-carboxamide; and
Example 88: N-(5-(3-((Cyanomethyl)carbamoyl)azetidin-1-yl)pentyl)-5-(4-fluorophenyl)isoxazole-3-carboxamide.

### Embodiment 12

A compound or a pharmaceutically acceptable salt thereof, according to Embodiment 1, wherein said compound is selected from any one or more exemplified examples.

### Embodiment 13

A pharmaceutical composition, comprising:

a therapeutically effective amount of a compound of Formula (I) according to any one of the Embodiments 1-12 or a pharmaceutically acceptable salt thereof, and

one or more pharmaceutically acceptable carriers.

### Embodiment 14

A pharmaceutical combination, comprising:

a therapeutically effective amount of a compound of Formula (I) according to any one of the Embodiments 1-12 or a pharmaceutically acceptable salt thereof, and

one or more therapeutically active agents.

### Embodiment 15

A method of treating hearing loss or balance disorder, comprising administering to a subject in need thereof a therapeutically effective amount of a compound according to any one of the Embodiments 1-12 or a pharmaceutically acceptable salt thereof.

### Embodiment 16

A method according to Embodiment 15, wherein the subject has a partial or complete loss of hearing.

### Embodiment 17

A method according to Embodiment 15 or 16, wherein the hearing loss is acquired hearing loss.

### Embodiment 18

A method according to any one of the Embodiments 15-17, wherein the hearing loss is sensorineural hearing loss.

### Embodiment 19

A method according to any one of the Embodiments 15-18, wherein the hearing loss or balance disorder is associated with damage or loss of sensory hair cells.

### Embodiment 20

A method according to any one of the Embodiments 15-19, wherein the hearing loss or balance disorder is caused by acute or chronic exposure to ototoxic compounds, acute or chronic exposure to noise, aging, autoimmune disease, physical trauma, inflammation or virus.

### Embodiment 21

A method according to any one of the Embodiments 15-20, wherein the compound or a pharmaceutically acceptable salt thereof, promotes, stimulates or induces sensory hair cells regeneration.

### Embodiment 22

A compound according to any one of the Embodiments 1-12, or a pharmaceutically acceptable salt thereof, for use as a medicament.

### Embodiment 23

A use of a compound according to any one of Embodiments 1-12, or a pharmaceutically acceptable salt thereof, in the manufacture of a medicament for the treatment of hearing loss or balance disorder.

Other features of the present disclosure should become apparent in the course of the above descriptions of exemplary embodiments that are given for illustration of the disclosure and are not intended to be limiting thereof.

### Definitions

For purposes of interpreting this specification, the following definitions will apply, and whenever appropriate, terms used in the singular will also include the plural. Terms used in the specification have the following meanings unless the context clearly indicates otherwise.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the present disclosure otherwise claimed.

The term “a,” “an,” “the” and similar terms used in the context of the present disclosure (especially in the context of the claims) are to be construed to cover both the singular and plural unless otherwise indicated herein or clearly contradicted by the context.

As used herein, the term “heteroatoms” refers to nitrogen (N), oxygen (O) or sulfur (S) atoms, in particular nitrogen or oxygen.

Unless otherwise indicated, any heteroatom with unsatisfied valences is assumed to have hydrogen atoms sufficient to satisfy the valences.

As used herein, the terms “alkyl” refers to a hydrocarbon radical of the general formula CₙH₂ₙ₊₁. The alkane radical may be straight or branched. For example, the term “C₁-C₆ alkyl” or “C₁ to C₆ alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like).

The term “C₀-C₆ alkylene” refers to a bond (when the number of carbon atom is 0) or a divalent alkylene group (may be straight or branched) containing 1 to 6 carbon atoms (e.g., methylene (—CH₂—), ethylene (—CH₂CH₂—), n-propylene (—CH₂CH₂CH₂—), iso-propylene (—CH(CH₃)CH₂—), n-butylene (—CH₂CH₂CH₂CH₂—), iso-butylene, tert-butylene, n-pentylene, isopentylene, neopentylene, n-hexylene and the like).

The term “alkoxy” refers to an alkyl linked to an oxygen, which may also be represented as —O—R or —OR, wherein the R represents the alkyl group. “C₁-C₆ alkoxy” or “C₁ to C₆ alkoxy” is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

“Halogen” or “halo” may be fluorine, chlorine, bromine or iodine (preferred halogens as substituents are fluorine and chlorine).

“Haloalkyl” is intended to include both branched and straight-chain saturated aliphatic hydrocarbon groups having the specified number of carbon atoms, substituted with one or more halogens. Thus, “C₁-C₆ haloalkyl” or “C₁ to C₆ haloalkyl” is intended to include, but not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl.

“Haloalkoxy” represents a haloalkyl group as defined above with the indicated number of carbon atoms attached through an oxygen bridge. For example, “C₁-C₆ haloalkoxy” or “C₁ to C₆ haloalkoxy” is intended to include, but not limited to, trifluoromethoxy, difluoromethoxy, 2,2,2-trifluoroethoxy, and pentafluorothoxy. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—.

The term “cycloalkyl” refers to nonaromatic carbocyclic ring that is fully hydrogenated ring, including mono-, bi- or poly-cyclic ring systems having the specified number of carbon atoms. Thus, “C₃-C₈ cycloalkyl” or “C₃ to C₈ cycloalkyl” is intended to include, but not limited to, cyclopropyl, cyclobutyl, cyclopentyl, cyclohexyl, and norbornyl.

The term “aryl” refers to 6- to 10-membered aromatic carbocyclic moieties having a single (e.g., phenyl) or a fused ring system (e.g., naphthalene.). A typical aryl group is phenyl group.

The term “heteroaryl” refers to aromatic moieties containing at least one heteroatom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, benzopyranyl, benzothiophenyl, benzoimidazolyl, benzoxazolyl, 1H-benzo[d][1,2,3]triazolyl, and the like.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a hetereoaryl fused to an aryl (e.g., phenyl).

The term “heterocyclyl” refers to a saturated or partially saturated, but not aromatic, ring or ring systems, which include a monocyclic ring, fused rings, bridged rings and spirocyclic rings having the specified number of ring atoms. For example, heterocyclyl includes, but not limited to, 5- to 6-membered heterocyclyl, 4- to 10-membered heterocyclyl, 4- to 14-membered heterocyclyl and 5- to 14-membered heterocyclyl. Unless otherwise specified, the heterocyclyl contain 1 to 7, 1 to 5, 1 to 3, or 1 to 2 heteroatoms independently selected from the group consisting of nitrogen, oxygen and sulphur as ring members, where the N and S can also optionally be oxidized to various oxidation states. The heterocyclic group can be attached at a heteroatom or a carbon atom. Examples of such heterocyclyl include, but are not limited to, azetidine, oxetane, piperidine, piperazine, pyrroline, pyrrolidine, imidazolidine, imidazoline, morpholine, tetrahydrofuran, tetrahydrothiophene, tetrahydrothiopyran, tetrahydropyran, 1,4-dioxane, 1,4-oxathiane, hexahydropyrimidinyl, 3-azabicyclo[3.1.0]hexane, azepane, 3-azabicyclo[3.2.2]nonane, decahydroisoquinoline, 2-azaspiro[3.3]heptane, 2-oxa-6-azaspiro[3.3]heptane, 2,6-diazaspiro[3.3]heptane, 8-aza-bicyclo[3.2.1]octane, 3,8-diazabicyclo[3.2.1]octane, 3-Oxa-8-aza-bicyclo[3.2.1]octane, 8-Oxa-3-aza-bicyclo[3.2.1]octane, 2-Oxa-5-aza-bicyclo[2.2.1]heptane, 2,5-Diazabicyclo[2.2.1]heptane, 1,4-dioxa-8-aza-spiro[4.5]decane, 3-oxa-1,8-diazaspiro[4.5]decane, octahydropyrrolo[3,2-b]pyrrol, and the like.

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may be unsubstituted or substituted with up to three R groups, and at each occurrence R is selected independently from the definition of R.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent.

Combinations of substituents and/or variables are permissible only if such combinations result in stable compounds.

As a person of ordinary skill in the art would be able to understand, for example, a ketone (—CH—C═O) group in a molecule may tautomerize to its enol form (—C═C—OH). Thus, this disclosure is intended to cover all possible tautomers even when a structure depicts only one of them.

The phrase “pharmaceutically acceptable” indicates that the substance or composition must be compatible chemically and/or toxicologically, with the other ingredients comprising a formulation, and/or the mammal being treated therewith.

Unless specified otherwise, the term “compounds of the present disclosure” refers to compounds of Formula (I) and subformulae thereof, as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

It will be recognized by those skilled in the art that the compounds of the present disclosure may contain chiral centers and as such may exist in different isomeric forms. As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)— or (S)—.

Geometric isomers may occur when a compound contains a double bond or some other feature that gives the molecule a certain amount of structural rigidity. If the compound contains a double bond, the substituent may be E or Z configuration. If the compound contains a disubstituted cycloalkyl, the cycloalkyl substituent may have a cis- or trans-configuration.

Conformational isomers (or conformers) are isomers that can differ by rotations about one or more a bonds. Rotamers are conformers that differ by rotation about only a single a bond.

The term “atropisomer” refers to a structural isomer based on axial or planar chirality resulting from restricted rotation in the molecule.

Unless specified otherwise, the compounds of the present disclosure are meant to include all such possible isomers, including racemic mixtures, optically pure forms and intermediate mixtures. Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. or other equivalent columns, using the appropriate solvent or mixture of solvents to achieve good separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated within the scope of the present disclosure.

As used herein, “pharmaceutically acceptable salts” refer to derivatives of the disclosed compounds wherein the parent compound is modified by making acid or base salts thereof. For example, pharmaceutically acceptable salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate or xinafoate salt form.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence the present disclosure further provides co-crystals comprising a compound of the present disclosure.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and idodine, such as ²H, ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation) or at least 6633.3 (99.5% deuterium incorporation).

Isotopically labeled compounds of this present disclosure can generally be prepared by conventional techniques known to those skilled in the art or by processes disclosed in the schemes or in the examples and preparations described below (or analogous process to those described herein), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

The term “solvate” means a physical association of a compound of this disclosure with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

As used herein, “polymorph(s)” refer to crystalline form(s) having the same chemical structure/composition but different spatial arrangements of the molecules and/or ions forming the crystals. Compounds of the present disclosure can be provided as amorphous solids or crystalline solids. Lyophilization can be employed to provide the compounds of the present disclosure as a solid.

The term “hearing loss” refers to a sudden or gradual decrease in how well a subject can hear.

The term “balance disorder” refers to disruption in the labyrinth (the inner ear organ) that controls the balance system, which allows a subject to know where his/her body is in the environment. Such disruption generally causes the subject to feel unsteady and/or dizzy.

The term “partial or complete hearing loss” refers to different degree of a decrease in the ability to perceive sounds.

The term “acquired hearing loss” refers to loss of hearing that occurs or develops some time during the lifespan but is not present at birth.

The term “sensorineural hearing loss” refers to hearing loss caused by damage to the sensory cells and/or nerve fibers of the inner ear.

As used herein, the term “patient” encompasses all mammalian species.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. Exemplary subjects include human beings of any age with risk factors for cancer disease.

As used herein, a subject is “in need of” a treatment if such subject would benefit biologically, medically or in quality of life from such treatment (preferably, a human).

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat’, “treating” or “treatment” of any disease/disorder refers the treatment of the disease/disorder in a mammal, particularly in a human, and include: (a) ameliorating the disease/disorder, (i.e., slowing or arresting or reducing the development of the disease/disorder, or at least one of the clinical symptoms thereof); (b) relieving or modulating the disease/disorder, (i.e., causing regression of the disease/disorder), either physically, (e.g., stabilization of a discernible symptom), physiologically, (e.g., stabilization of a physical parameter), or both); (c) alleviating or ameliorating at least one physical parameter including those which may not be discernible by the subject; and/or (d) preventing or delaying the onset or development or progression of the disease or disorder from occurring in a mammal, in particular, when such mammal is predisposed to the disease or disorder but has not yet been diagnosed as having it.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to at least partially alleviate, inhibit, prevent and/or ameliorate hearing loss and/or balance disorder.

The effective amount can vary depending on such factors as the size and weight of the subject, the type of illness, or the particular compound of the present disclosure. One of ordinary skill in the art would be able to study the factors contained herein and make the determination regarding the effective amount of the compounds of the present disclosure without undue experimentation.

The regimen of administration can affect what constitutes an effective amount. The compound of the present disclosure can be administered to the subject either prior to or after the onset of hearing loss and/or balance disorder. Further, several divided dosages, as well as staggered dosages, can be administered daily or sequentially, or the dose can be continuously infused, or can be a bolus injection. Further, the dosages of the compound(s) of the present disclosure can be proportionally increased or decreased as indicated by the exigencies of the therapeutic or prophylactic situation.

### Preparation of Compounds

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd}-ed., Wiley-VCH Weinheim, Germany (1999), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

The following abbreviations used herein below have the corresponding meanings:

| | |
|---|---|
| CDI | di(1H-imidazol-1-yl)methanone |
| CH₃CN/MeCN | acetonitrile |
| CH₃MgBr | methyl magnesium bromide |
| CH₃NH₂ | methanamine |
| (COCl)₂ | oxalyl dichloride |
| (COOEt)₂ | diethyl oxalate |
| CuI | copper(I) iodate |
| DCM/CH₂Cl₂ | dichloromethane |
| DIAD | diisopropyl azodiformate |
| DIEA/DIPEA | N-ethyl-N-isopropylpropan-2-amine |
| DMF | dimethylformamide |
| DMP | Dess-Martin periodinane |
| DMSO | dimethylsulfoxide |
| EDCl | 1-(3-dimethylaminopropyl)-3-ethylcarbodiimide |
| | hydrochloride |
| Et₃N | triethylamine |
| EtOAc | ethyl acetate |
| EtOH | ethanol |
| H₂ | hydrogen |
| H₂O | water |
| HAUT | 2-(7-aza-1H-benzotriazole-1-yl)-1,1,3,3- |
| | tetramethyluronium hexafluorophosphate |
| HCl | hydrochloric acid |
| HOAc | acetic acid |
| HOBt | 1-Hydroxybenzotriazole |
| HPLC | high performance liquid chromatography |
| K₂CO₃ | potassium carbonate |
| KI | Potassium iodide |
| LiOH•H₂O | lithium hydroxide hydrate |
| m-CPBA | 3-chloroperoxybenzoic acid |
| Me₃Al | trimethylaluminium |
| MeOH | methanol |
| MgSO₄ | magnesium sulphate |
| mL | millilitre |
| MS | mass spectrometer |
| MsCl | methanesulfonyl chloride |
| N₂ | nitrogen |
| NaBH₃CN | sodium cyanoborohydride |
| NaB(OAc)₃H | sodium triacetoxyhydroborate |
| NaHCO₃ | sodium bicarbonate |
| Na₂SO₄ | sodium sulfate |
| Na₂SO₃ | sodium sulfite |
| NH₃•H₂O/NH₄OH | ammonia |
| NH₂OH•HCl | hydroxylamine hydrochloride |
| NBS | N-Bromosuccinimide |
| Pd(OH)₂/C | palladium hydroxide on carbon |
| PPh₃ | triphenylphosphine |
| rt | room temperature |
| t-BuOK | potassium tert-butoxide |
| TFA | trifluoroacetic acid |
| THF | tetrahydrofuran |

### LC/MS Methods Employed in Characterization of Examples

LC/MS data were recorded using Agilent 1100 HPLC systems with Waters Micromass ZQ, or Waters ACQUITY UPLC with Waters SQ detector or with Waters ACQUITY QDa detector.

### NMR Employed in Characterization of Examples

¹H NMR spectra were obtained with Bruker Fourier transform spectrometers operating at frequencies as follows: ¹H NMR: 400 MHz (Bruker). ¹³C NMR: 100 MHz (Bruker). Spectra data are reported in the format: chemical shift (multiplicity, number of hydrogens). Chemical shifts are specified in ppm downfield of a tetramethylsilane internal standard (δ units, tetramethylsilane=0 ppm) and/or referenced to solvent peaks, which in ¹H NMR spectra appear at 2.50 ppm for CD₃SOCD₃, 3.31 ppm for CD₃OD, 1.94 for CD₃CN, 4.79 for D₂O, 5.32 for CD₂Cl₂, and 7.26 ppm for CDCl₃, and which in ¹³C NMR spectra appear at 39.7 ppm for CD₃SOCD₃, 49.0 ppm for CD₃OD, 1.32 and/or 118.26 for CD₃CN, 53.84 for CD₂Cl₂, and 77.0 ppm for CDCl₃. All ¹³C NMR spectra were proton decoupled.

### Methods Employed in the Purification of the Examples

Purification of intermediates and final products was carried out via either normal or reverse phase chromatography. Normal phase chromatography was carried out using prepacked SiO₂ cartridges (e.g., RediSep® Rf columns from Teledyne Isco, Inc.) eluting with gradients of appropriate solvent systems (e.g., hexanes and ethyl acetate; DCM and MeOH; or unless otherwise indicated). Reverse phase preparative HPLC was carried out using the methods described in individual example experimental procedure with corresponding information on column, basic/neutral/acidic condition, and acetonitrile gradient range.

### General Synthetic Schemes

Schemes 1-4 (shown below) describe potential routes for preparing the compounds of the present disclosure which include compounds of Formula (I) and subformulae thereof. The starting materials for the below reaction scheme are commercially available or can be prepared according to methods known to one skilled in the art or by methods disclosed herein. Compounds of Formula (I) can be made substantially optically pure by either using substantially optically pure starting material or by separation chromatography, recrystallization or other separation techniques well-known in the art. For a more detailed description, see the Example section below.

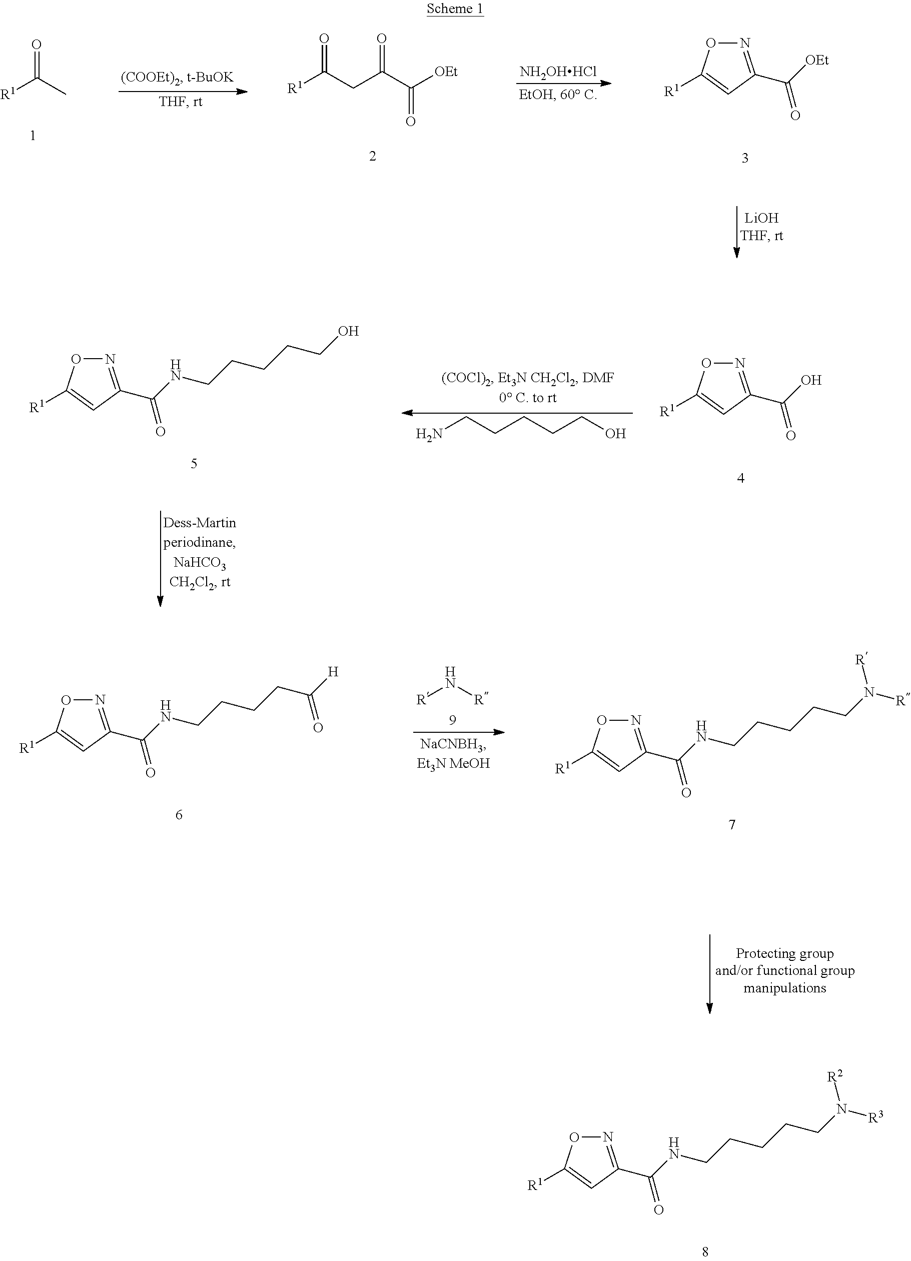

As depicted in scheme 1, aromatic methyl ketone 1 is treated with strong base (such as t-BuOK) and diethyl oxalate to yield α-ketyl ester 2, which cyclizes with hydroxylamine hydrochloride to give isoxazole ester 3. Subsequent hydrolysis of compound 3 by LiOH furnishes acid 4, which is converted to the corresponding acid chloride via oxalyl chloride and then couples with 5-aminopentan-1-ol to generate amide 5. The alcohol of compound 5 is further oxidized by Dess-Martin periodinane to give aldehyde 6, which undergoes reductive amination with various amine 9 (R′ and R″ each represent various substitutents on the N of the amine 9) in the presence of NaCNBH₃ or NaBH(OAc)₃ to generate corresponding tertiary amine 7. Depending on the structure of amine 9, compound 7 can go through protecting group and/or functional group manipulations to provide target molecule 8.

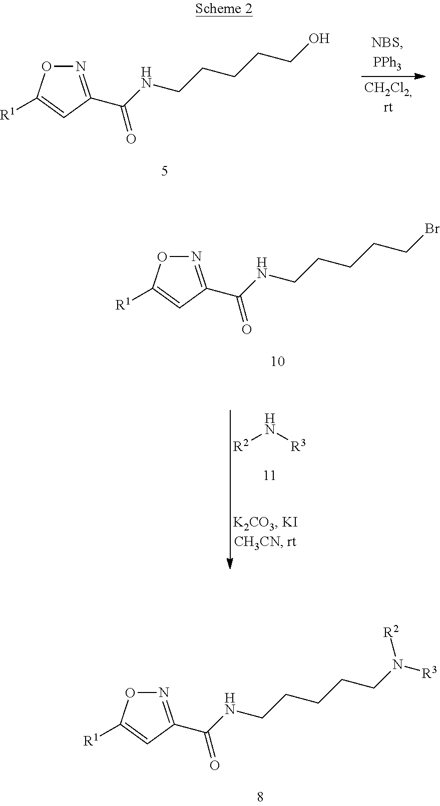

Alternatively in Scheme 2, alcohol 5 is converted to the corresponding bromide 10 via NBS, which undergoes alkylation with various amines 11 in the presence of weak base (such as K₂CO₃) to provide the target molecule 8.

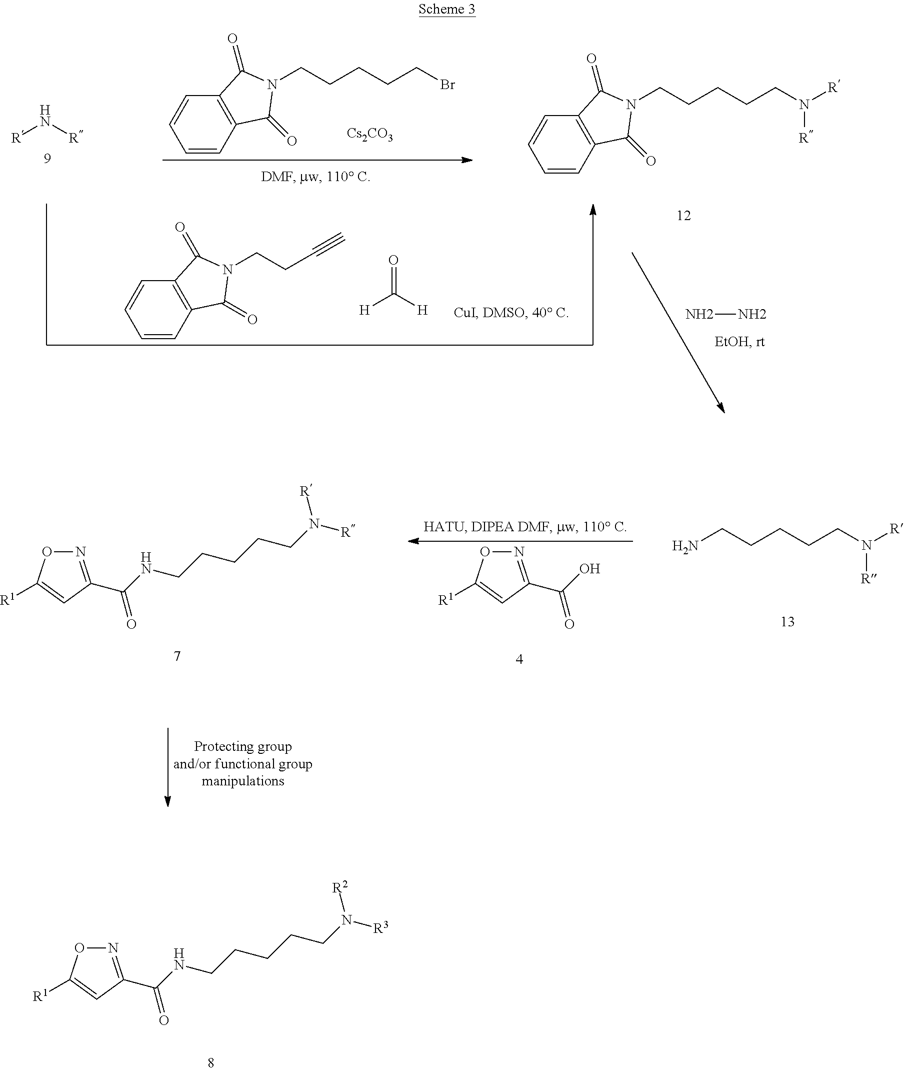

In addition, as shown in Scheme 3, secondary amine 9 (R′ and R″ each represent various substitutents on the N of the amine 9) either undergoes alkylation in the presence of base (such as Cs₂CO₃) with 2-(5-bromopentyl)isoindoline-1,3-dione, or goes through three component coupling reaction with 2-(but-3-yn-1-yl)isoindoline-1,3-dione and formaldehyde in the presence of catalytic copper iodide to form tertiary amine 12. Compound 12 is de-protected with hydrazine to provide primary amine 13, which then reacts with acid 4 under general amide coupling conditions (such as HATU, EDCI/HOBt, etc.) to provide tertiary amine 7. Depending on the structure of amine 9, compound 7 can go through protecting group and/or functional group manipulations to provide target molecule 8.

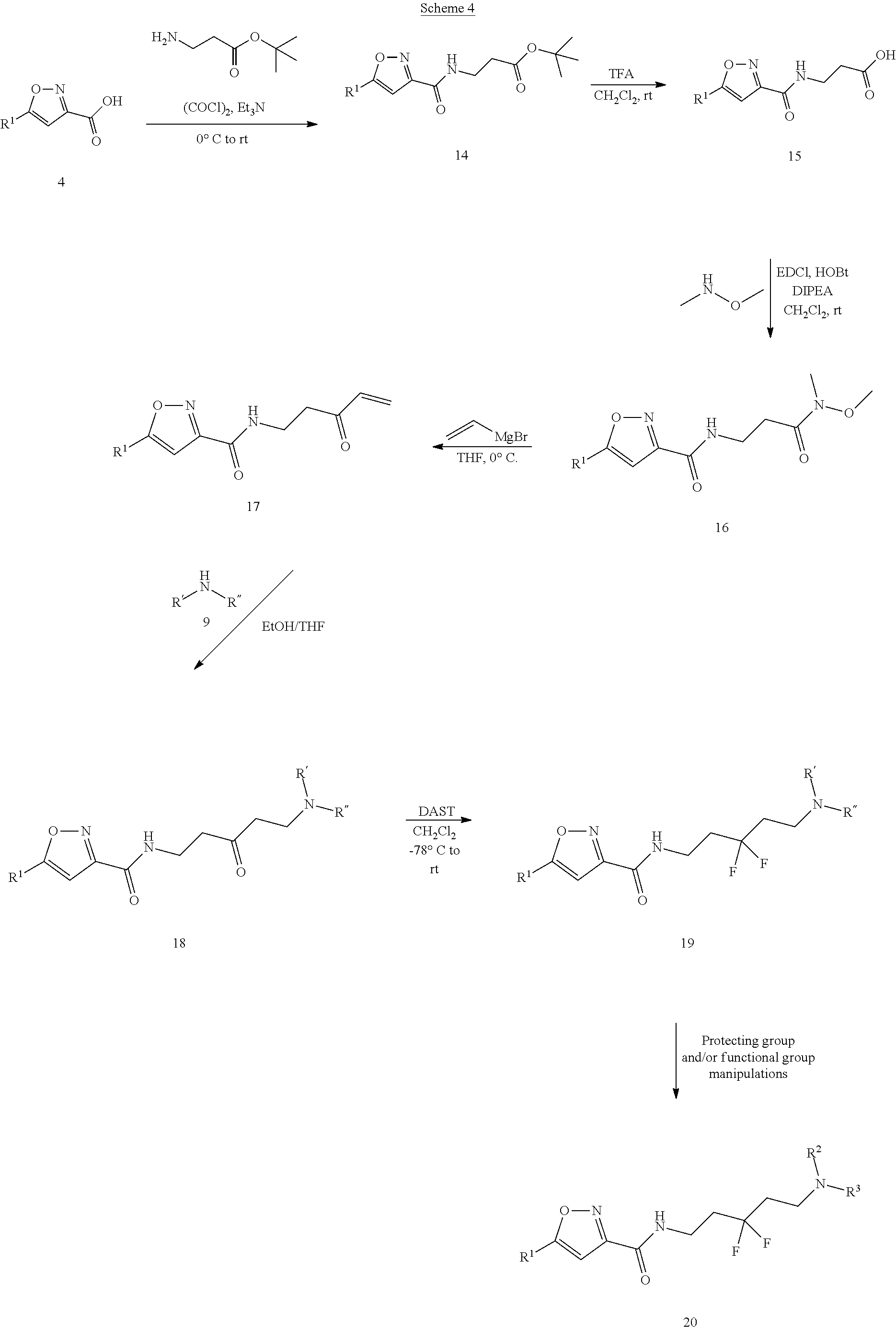

As illustrated in Scheme 4, acid 4 is converted to corresponding acid chloride via oxalyl chloride and then couples with tert-butyl 3-aminopropanoate to yield amide 14, which is hydrolyzed under acidic conditions (such as TFA) to generate acid 15. Compound 15 is converted to Weinreb amide 16 under general amide coupling conditions (such as EDCI/HOBt, HATU, etc.) with N, O-dimethyl hydroxylamine. Compound 16 undergoes nucleophilic addition with vinyl Grignard to form α, β unsaturated ketone 17, which functions as Michael acceptor and can be added by various amines 9 (R′ and R″ each represent various substitutents on the N of the amine 9) to form β-ketyl amine 18. The carbonyl group of compound 18 undergoes fluorination via DAST to provide compound 19, which can go through protecting and/or functional group manipulations to provide the di-F substituted target molecule 20.

### EXAMPLES

The following Examples have been prepared, isolated and characterized using the methods disclosed herein. The following examples demonstrate a partial scope of the disclosure and are not meant to be limiting of the scope of the disclosure.

Unless specified otherwise, starting materials are generally available from a non-limiting commercial sources such as TCI Fine Chemicals (Japan), Shanghai Chemhere Co., Ltd. (Shanghai, China), Aurora Fine Chemicals LLC (San Diego, Calif.), FCH Group (Ukraine), Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), AstraZeneca Pharmaceuticals (London, England), Chembridge Corporation (USA), Matrix Scientific (USA), Conier Chem & Pharm Co., Ltd (China), Enamine Ltd (Ukraine), Combi-Blocks, Inc. (San Diego, USA), Oakwood Products, Inc. (USA), Apollo Scientific Ltd. (UK), Allichem LLC. (USA) and Ukrorgsyntez Ltd (Latvia).

### INTERMEDIATES

### Intermediate A: 5-(Thiophen-2-yl)isoxazole-3-carboxylic Acid

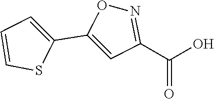 [SMILES: O=C(O)c1cc(-c2cccs2)on1]

### Step 1: Ethyl 2,4-dioxo-4-(thiophen-2-yl)butanoate

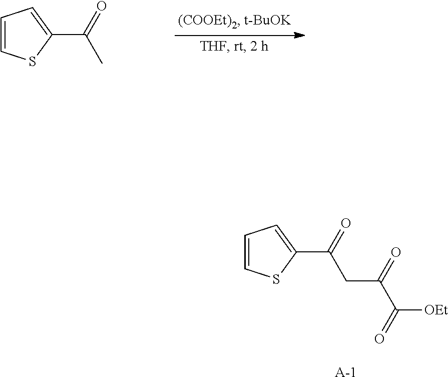

To a solution of 1-(thiophen-2-yl)ethan-1-one (50 g, 396.2 mmol, 1.0 eq) and (COOEt)₂ (72.39 g, 495.3 mmol, 1.25 eq) in anhydrous THF (2.0 L) was added t-BuOK (57.8 g, 515.1 mmol, 1.3 eq) in small portions at 15-25° C. Then the mixture was stirred at rt for 2 hours. The mixture was poured into water (800 mL), acidified to pH 2 with 1N HCl, and then the mixture was extracted with ethyl acetate (3*500 mL). The organic layer was separated and washed with brine (1 L), dried over anhydrous sodium sulfate, and concentrated to give the crude title product (100 g) as a yellow solid which was used without further purification.

### Step 2: Ethyl 5-(thiophen-2-yl)isoxazole-3-carboxylate

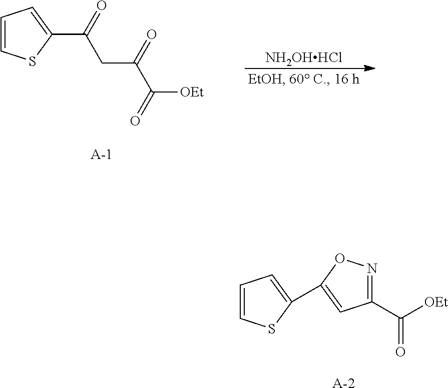 [SMILES: CCOC(=O)C(=O)CC(=O)c1cccs1 ; "NH2OH·HCl" ; "A-1" ; CCOC(=O)c1cc(-c2cccs2)on1 ; "A-2"]

To a solution of compound A-1 (89 g, 393.3 mmol, 1.0 eq) in anhydrous ethanol (2 L) was added compound NH₂OH.HCl (54.64 g, 786.7 mmol, 2 eq). The mixture was stirred at 60° C. for 16 hours. The reaction mixture was concentrated. Water (200 mL) was added and the mixture was extracted with EtOAc (3*200 mL). The organic layer was concentrated under the vacuum to afford the crude title product (90 g) which was used without further purification.

### Step 3: 5-(Thiophen-2-yl)isoxazole-3-carboxylic Acid

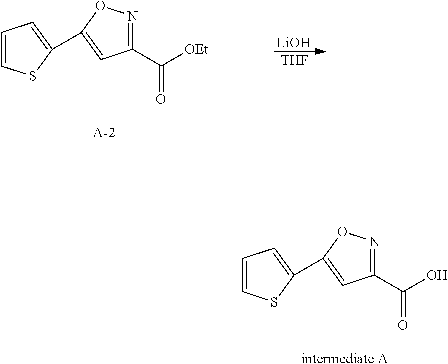 [SMILES: CCOC(=O)c1cc(-c2cccs2)on1 ; "LiOH" ; "A-2" ; O=C(O)c1cc(-c2cccs2)on1 ; "intermediate A" ; "THF"]

To a solution of compound A-2 (80 g, 358.3 mmol, 1.0 eq) in THF (200 mL) was added a solution of LiOH.H₂O (17.16 g, 716.6 mmol, 2.0 eq) in water (358.3 mL). The resulting mixture was stirred at 15-22° C. for 2 hours. The reaction mixture was concentrated under reduced pressure to remove THF. The residue was acidified to pH 1 with 1 N HCl and extracted with EtOAc (3*300 mL). The combined organic layers were concentrated under the vacuum. The solid was triturated with EtOAc, filtered and dried to give the title compound (42.6 g, 60.9% yield) as a white solid.

¹H NMR (400 MHz, CDCl₃) δ ppm 7.60-7.59 (dd, J=3.6, 1.2 Hz, 1H), 7.54-7.52 (dd, J=4.8, 1.2 Hz, 1H), 7.18-7.16 (dd, J=4.8, 3.6 Hz, 1H), 6.84 (s, 1H).

### Intermediate B: N-(5-Oxopentyl)-5-(thiophen-2-yl)isoxazole-3-carboxamide

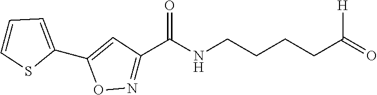 [SMILES: O=CCCCCNC(=O)c1cc(-c2cccs2)on1]

### Step 1: N-(5-Hydroxypentyl)-5-(thiophen-2-yl)isoxazole-3-carboxamide

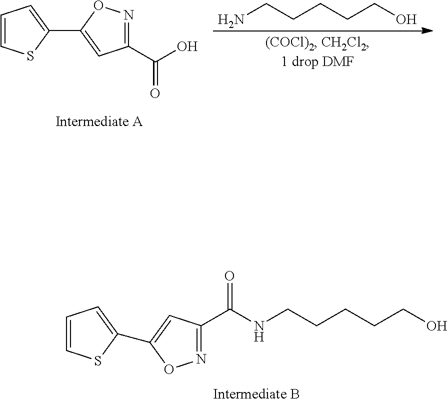

To a solution of compound Intermediate A (10 g, 51.23 mmol, 1.0 eq) in anhydrous CH₂Cl₂ (100 mL) was added (COCl)₂ (19.5 g 13.1 mL, 153.6 mmol, 3.0 eq) dropwise under N₂ protection, then one drop DMF was added at 0° C. The mixture was stirred at rt for 2 hours. Then the mixture was concentrated under the vacuum and the residue was diluted with CH₂Cl₂ (50 mL), then the mixture was added to a solution of 5-aminopentan-1-ol (7.93 g, 76.85 mmol, 1.5 eq) and Et₃N (15.5 g, 153.69 mmol, 3.0 eq) in CH₂Cl₂ (100 mL) dropwise at 0° C. The resulted mixture was stirred at rt for 1 hour. Then the reaction was quenched with water (50 mL) and extracted with CH₂Cl₂ (3*50 mL). The organic layers were dried over anhydrous Na₂SO₄, filtered and concentrated under the vacuum to afford the title compound (12.5 g, 87.03% yield) as a white solid.

MS (ESI) m/z 302.9 [M+Na]⁺.

### Step 2: N-(5-Oxopentyl)-5-(thiophen-2-yl)isoxazole-3-carboxamide

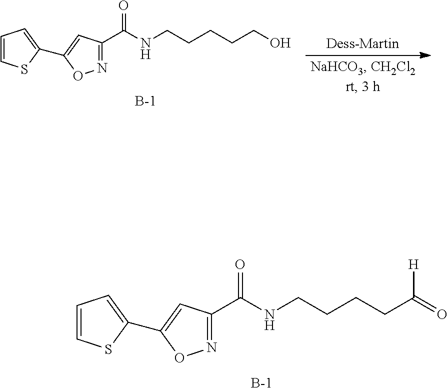

To a solution of compound B-1 (10 g, 35.67 mmol, 1.0 eq) in CH₂Cl₂ (200 mL) was added NaHCO₃ (13.48 g, 160.5 mmol, 4.5 eq), followed by DMP (22.69 g, 53.5 mmol, 1.5 eq). The resulting mixture was stirred at rt for 3 hours. The mixture was slowly poured into saturated NaHCO₃ aqueous solution (100 mL) and extracted with CH₂Cl₂ (3*100 mL). The combined organic layers were dried over anhydrous sodium sulfate, filtered and concentrated in vacuum and the residue was purified by silica gel chromatography eluting with petroleum/EtOAc from 100/0 to 1/1 to give the title compound (4.5 g, 45.3% yield) as a white solid. MS (ESI) m/z 300.9 [M+Na]⁺.

### Intermediate C: 5-(4-Fluorophenyl)-N-(5-oxopentyl)isoxazole-3-carboxamide

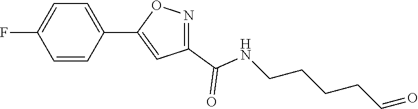 [SMILES: O=CCCCCNC(=O)c1cc(-c2ccc(F)cc2)on1]

The title compound was prepared by using a procedure similar to that of Intermediate of B by replacing of intermediate A with 5-(4-fluorophenyl)isoxazole-3-carboxylic acid (which was made using the similar method as intermediate A) in 28% yield as a white solid. MS (ESI) m/z 312.9 [M+H]⁺. ¹H NMR (400 MHz, CDCl₃) δ ppm 9.81 (t, J=1.2 Hz, 1H), 7.82-7.78 (m, 2H), 7.23-7.16 (m, 2H), 6.92 (s, 1H), 3.50 (q, J=6.4 Hz, 2H), 2.59-2.50 (m, 2H), 1.80-1.64 (m, 4H).

### Intermediate D: N-Methylazetidine-3-carboxamide

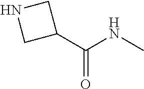

### Step 1: 1-Benzhydryl-N-methylazetidine-3-carboxamide

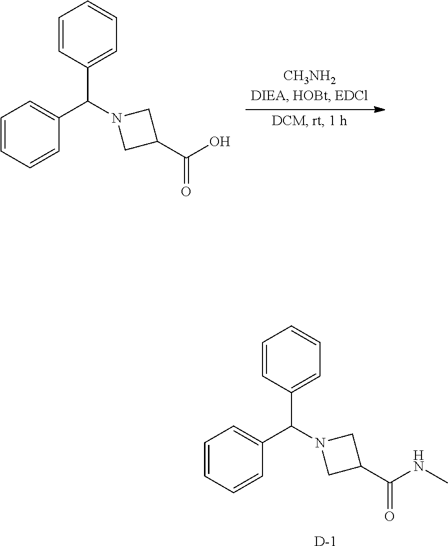

To a solution of 1-benzhydrylazetidine-3-carboxylic acid (4.0 g, 14.96 mmol, 1.0 eq), and CH₃NH₂ (8.98 mL, 17.96 mmol, 1.2 eq, 2 M in THF) in CH₂Cl₂ (60 mL) was added EDCI (5.74 g, 29.93 mmol, 2.0 eq), HOBt (3.03 g, 22.44 mmol, 1.5 eq) and DIEA (9.89 mmol, 59.85 mmol, 4.0 eq) sequently. The resulting mixture was stirred at 23° C. for 1 hour. The mixture was diluted with water (60 mL), then the organic phase was washed with brine (3*60 mL), dried over anhydrous Na₂SO₄, filtered and concentrated. The crude product was purified by silica gel chromatography eluting with DCM/methanol to give the title compound (3.70 g, 88.2% yield) as a white solid. ¹H NMR (400 MHz, CDCl₃) δ ppm 7.36-7.34 (m, 4H), 7.24-7.22 (m, 4H), 7.18-7.14 (m, 2H), 6.06 (s, 1H), 4.39 (s, 1H), 3.32 (t, J=8.0 Hz, 2H), 3.25 (t, J=6.0 Hz, 2H), 3.06-2.98 (m, 1H), 2.82 (d, J=4.8 Hz, 3H).

### Step 2: N-Methylazetidine-3-carboxamide

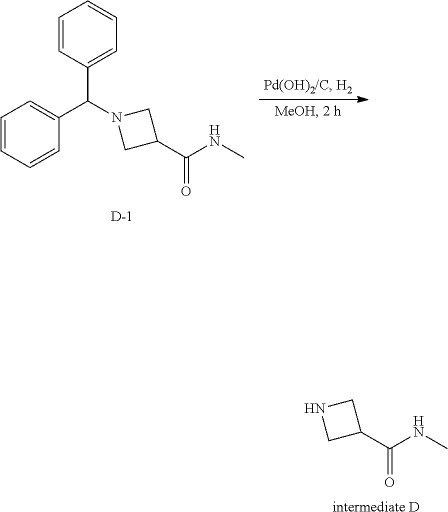

To a solution of intermediate D-1 (3.0 g, 10.70 mmol, 1.0 eq) in methanol (50 mL) was added Pd(OH)/C (300 mg, 10% wt) and the resulting mixture was stirred at 50° C. under H₂ (50 psi) for 12 hours. The mixture was filtered and the filtrate was concentrated and the crude product was purified by silica gel chromatography eluting with DCM/methanol to give the title compound (1.10 g, 90.1% yield) as a brown oil. ¹H NMR (CDCl₃, 400M Hz) δ ppm 6.29 (s, 1H), 5.05 (s, 1H), 3.84 (t, J=8.0 Hz, 2H), 3.65 (t, J=8.4 Hz, 2H), 3.37-3.29 (m, 1H), 2.79 (d, J=4.8 Hz, 3H).

### Intermediate E: N-Cyclopropylazetidine-3-carboxamide

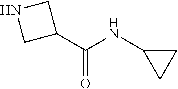

The title compound was prepared by using a procedure similar to that of Intermediate of D by replacing of methyl amine with cyclopropanamine as a light yellow oil. MS (ESI) m/z 141.0 [M+H]⁺.

### Intermediate F: 3-(Methylsulfonyl)azetidine

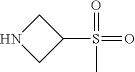

### Step 1: tert-Butyl 3-((methylsulfonyl)oxy)azetidine-1-carboxylate

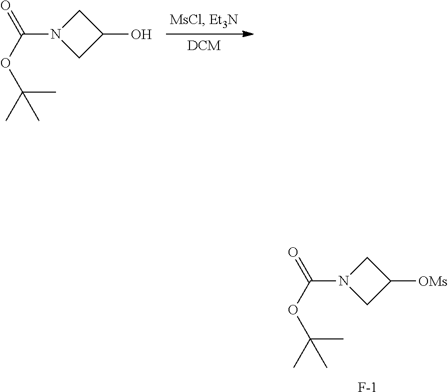

To a solution of tert-butyl 3-hydroxyazetidine-1-carboxylate (3.0 g, 17.32 mmol, 1.0) in CH₂Cl₂ (40 mL) was added Et₃N (2.63 g, 25.98 mmol, 1.5 eq), and then MsCl (2.38 g, 20.78 mmol, 1.2 eq) was added at 0° C. The mixture was stirred at rt for 14 hours. The reaction mixture was diluted with CH₂Cl₂ (40 mL). The organic phase was washed with water (40 mL), 1.0 N HCl (20 mL) and brine (20 mL) successively. The organic layer was dried over Na₂SO₄, filtered and concentrated under reduced pressure to give the crude title compound (4.2 g, 96.5% yield) as a light yellow oil which was used without further purification.

### Step 2: tert-Butyl 3-(methylthio)azetidine-1-carboxylate

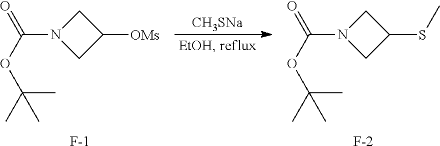

To a solution of compound F-1 (2.17 g, 8.64 mmol, 1.0 eq) in EtOH (12 mL) was added sodium methanethiolate (907.8 mg, 12.9 mmol, 1.5 eq). The mixture was heated under reflux for 2 hours. The mixture was diluted with water (30 mL). The aqueous phase was extracted with EtOAc (3*20 mL). The combined organic phase was dried over Na₂SO₄, filtered. The filtrate was concentrated under reduced pressure. The residue was purified by silica gel chromatography eluting with petroleum/EtOAc from 50/1 to 5/1 to give the title compound (1.2 g, 68% yield) as a light yellow oil. ¹H NMR (400 MHz, CDCl₃) δ ppm 4.23 (t, J=8.8 Hz, 1H), 3.84-3.81 (m, 2H), 3.58-3.54 (m, 1H), 2.11 (s, 3H), 1.43 (s, 9H).

### Step 3: tert-Butyl 3-(methylsulfonyl)azetidine-1-carboxylate

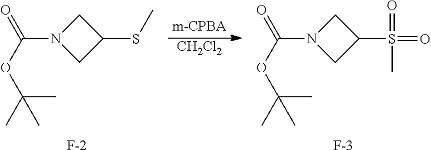

To a ice-cooled solution of compound F-2 (0.7 g, 3.44 mmol, 1.0 eq) in CH₂Cl₂ (10 mL) was added m-CPBA (1.54 g, 7.57 mmol, 2.2 eq) in small portions at 0-5° C. The mixture was stirred at 0° C. for 3 hours. The mixture was quenched with saturated NaHCO₃ aqueous solution (20 mL). The organic phase was washed with saturated Na₂SO₃ aqueous solution (2*20 mL), dried over Na₂SO₄, and filtered. The filtrate was concentrated under reduced pressure and the residue was purified by silica gel chromatography eluting with petroleum/EtOAc to afford the title compound (0.6 g, 74% yield) as an off-white solid. ¹H NMR (400 MHz, CDCl₃) δ ppm 4.26-4.19 (m, 4H), 3.91-3.89 (m, 1H), 2.90 (s, 3H), 1.44 (s, 9H).

### Step 4: 3-(Methylsulfonyl)azetidine

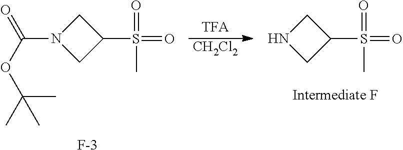

To a solution of compound F-3 (0.6 g, 2.55 mmol, 1.0 eq) in CH₂Cl₂ (4 mL) was added TFA (1.48 g, 13.0 mmol, 5.1 eq) at 25° C. The mixture was stirred at 25° C. for 14 hours. The volatile was removed under reduced pressure to afford the crude title compound which was directly used in the next step.

### Intermediate G: Azetidine-3-sulfonamide

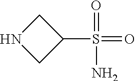

### Step 1: Benzyl 3-(acetylthio)azetidine-1-carboxylate

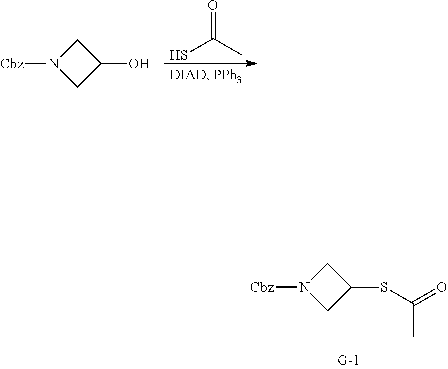

To a solution of PPh₃ (7.91 g, 30.16 mmol, 1.25 eq) in THF (30 mL) at −78° C. was added DIAD (5.95 g, 29.44 mmol, 1.22 eq) in THF (20 mL). After stirred for 10 min, thioacetic acid (2.39 g, 2.24 mL, 31.37 mmol, 1.3 eq) in THF (20 mL) was added. After additional 10 min, a solution of benzyl 3-hydroxyazetidine-1-carboxylate (5 g, 24.13 mmol, 1.0 eq) in THF (30 mL) was added. The reaction was stirred at −78° C. for 1 hour and then allowed to warm to 25° C. for 14 hours. The reaction mixture was quenched with brine (30 mL). The aqueous phase was extracted with EtOAc (3*20 mL). The combined organic phase was dried over Na₂SO₄, filtered and concentrated under reduced pressure. The residue was purified by silica gel column chromatography eluting with petroleum/EtOAc from 50/1 to 5/1 to afford the title compound (2.0 g, 31% yield) as a light yellow oil.

¹H NMR (400 MHz, CDCl₃) δ ppm 7.38-7.28 (m, 5H), 5.11 (s, 2H), 4.49-4.45 (m, 2H), 4.24-4.21 (m, 1H), 3.94-3.90 (m, 2H), 2.35 (s, 3H).

### Step 2: Benzyl 3-(chlorosulfonyl)azetidine-1-carboxylate

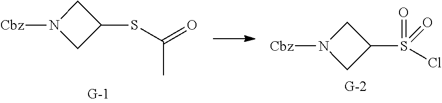 [SMILES: CC(=O)SC1CN(C(=O)OCc2ccccc2)C1 ; O=C(OCc1ccccc1)N1CC(S(=O)(=O)Cl)C1]

To a solution of compound G-1 (1.1 g, 4.15 mmol, 1.0 eq) in CH₂Cl₂ (20 mL) was added water (5 mL). The mixture was cooled to 0° C. and chlorine gas was bubbled through at 0-5° C. with stirring for 1 hour. The layers were separated and the DCM layer containing compound G-2 (4.15 mmol) was used directly in the next step.

### Step 3: Benzyl 3-sulfamoylazetidine-1-carboxylate

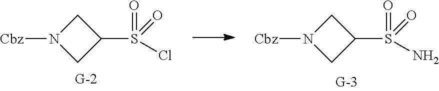 [SMILES: O=C(OCc1ccccc1)N1CC(S(=O)(=O)Cl)C1 ; NS(=O)(=O)C1CN(C(=O)OCc2ccccc2)C1]

To a solution of NH₃.H₂O (40 mL, 0.34 mol, 28% wt, 82.7 eq) was added a solution of compound G-2 (4.15 mmol, 1.0 eq) in CH₂Cl₂ (20 mL) at 0-5° C. The mixture was stirred at 26° C. for 14 hours. The aqueous phase was extracted with CH₂Cl₂ (2*40 mL). The combined organic phase was dried over Na₂SO₄, filtered, concentrated. The residue was purified by acidic preparative HPLC (Boston Green ODS 150*30 5 u, gradient: 22-32% B (A=0.1% TFA/water), B=CH₃CN), flow rate: 30 mL/min) to afford the title compound (0.35 g, 31.2% yield) as a light yellow solid. MS (ESI) m/z 292.9 [M+23]⁺. ¹H NMR (400 MHz, CDCl₃) δ ppm 7.36-7.31 (m, 5H), 5.13 (s, 2H), 5.10 (s, 2H), 4.32-4.22 (m, 4H), 4.02-4.00 (m, 1H).

### Step 4: Azetidine-3-sulfonamide

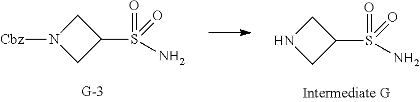 [SMILES: NS(=O)(=O)C1CN(C(=O)OCc2ccccc2)C1 ; NS(=O)(=O)C1CNC1]

To a solution of compound G-3 (0.35 g, 1.29 mmol, 1.0 eq) in MeOH (3 mL) was added Pd/C (0.1 g, 10% wt). The mixture was stirred at 25° C. under hydrogen atmosphere (15 psi) for 4 hours. The mixture was filtered, and the cake was washed with MeOH (2*5 mL). The filtrate was concentrated to give the title compound (160 mg, 90.7% yield) as a light yellow solid. MS (ESI) m/z 136.9 [M+1]⁺. ¹H NMR (400 MHz, DMSO-d₆) δ ppm 6.90 (brs, 2H), 4.10-4.04 (m, 1H), 3.74-3.70 (m, 2H), 3.60-3.56 (m, 2H).

### Example 1: N-(5-(3-Phenylpiperazin-1-yl)pentyl)-5-(thiophen-2-yl)isoxazole-3-carboxamide

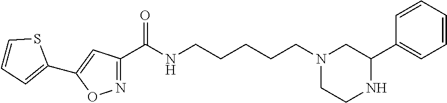 [SMILES: O=C(NCCCCCN1CCNC(c2ccccc2)C1)c1cc(-c2cccs2)on1]

### Step 1: Preparation of tert-Butyl 4-(5-(1,3-dioxoisoindolin-2-yl)pentyl)-2-phenylpiperazine-1-carboxylate

In a microwave vial, tert-butyl 2-phenylpiperazine-1-carboxylate (500 mg, 1.906 mmol, 1 eq), cesium carbonate (1863 mg, 5.72 mmol, 3 eq), and 2-(5-bromopentyl)isoindoline-1,3-dione (564 mg, 1.906 mmol, 1 eq) were dissolved in DMF (3 mL). The reaction was put in the microwave for 25 min at 110° C. The mixture was taken up in EtOAc and water extracted with EtOAc. The combined organics were washed with brine, dried over MgSO₄, filtered, and concentrated, and purified by silica gel chromatography to give the title compound (460 mg, 50.5% yield) as a colorless oil.

### Step 2. Preparation of tert-Butyl 4-(5-aminopentyl)-2-phenylpiperazine-1-carboxylate

A solution of tert-butyl 4-(5-(1,3-dioxoisoindolin-2-yl)pentyl)-2-phenylpiperazine-1-carboxylate (450 mg, 0.942 mmol, 1 eq), and hyrazine (0.148 mL, 4.71 mmol, 5 eq) in EtOH (10 mL) was stirred at rt overnight. The mixture was concentrated and the residue was triturated with DCM, filtered and the filtration was concentrated to give the title compound as a white solid which was used without further purification.

### Step 3: Preparation of tert-Butyl 2-phenyl-4-(5-(5-(thiophen-2-yl)isoxazole-3-carboxamido)pentyl)piperazine-1-carboxylate

5-(Thiophen-2-yl)isoxazole-3-carboxylic acid (172 mg, 0.883 mmol, 1 eq) was dissolved in DMF to which HATU (403 mg, 1.060 mmol, 1.2 eq) was added. Then a solution of the DIPEA (617 μl, 3.53 mmol, 4 eq) and tert-butyl 4-(5-aminopentyl)-2-phenylpiperazine-1-carboxylate (307 mg, 0.883 mmol, 1 eq) in DMF (2 mL) was added. The microwave vial was capped and put in the microwave for 15 minutes at 110° C. The reaction mixture was taken up in EtOAc and washed several times with water. The combined organics were washed with brine, dried over MgSO₄, filtered, and rotary evaporated, and purified by silica gel chromatography eluting with heptane/EtOAc to give the title compound which was used without further purification.

### Step 4. Preparation of N-(5-(3-Phenylpiperazin-1-yl)pentyl)-5-(thiophen-2-yl)isoxazole-3-carboxamide

tert-Butyl 2-phenyl-4-(5-(5-(thiophen-2-yl)isoxazole-3-carboxamido)pentyl)piperazine-1-carboxylate (258 mg, 0.492 mmol, 1 eq) was dissolved in DCM (5 mL) to which TFA (0.758 mL, 9.83 mmol, 20 eq) was added. The reaction mixture was stirred at rt for several hours, then rotary evaporated and purified by neutral preparative HPLC to give the title compound (98.23 mg, 47.1% yield). MS (ESI) m/z 425.3 [M+H]⁺. ¹H NMR (400 MHz, DMSO-d₆) δ ppm 8.53 (t, J=5.56 Hz, 1H), 7.84 (dd, J=5.05, 1.01 Hz, 1H), 7.75 (dd, J=3.79, 1.26 Hz, 1H), 7.39-7.29 (m, 5H), 7.25 (dd, J=5.05, 3.54 Hz, 1H), 7.07 (s, 1H), 3.41 (dd, J=11.37, 2.78 Hz, 1H), 3.32 (d, J=11.62 Hz, 1H), 3.22-3.02 (m, 6H), 2.92-2.83 (m, 1H), 2.42-2.27 (m, 2H), 1.94 (dt, J=12.63, 6.32 Hz, 1H), 1.46-1.28 (m, 4H), 1.27-0.99 (m, 2H).

### Example 2: tert-Butyl 4-(5-(5-(thiophen-2-yl)isoxazole-3-carboxamido)pentyl)piperazine-1-carboxylate

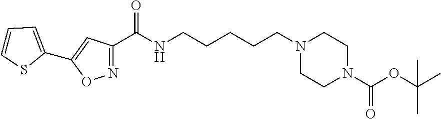 [SMILES: CC(C)(C)OC(=O)N1CCN(CCCCCNC(=O)c2cc(-c3cccs3)on2)CC1]

The title compound was prepared by using a procedure similar to that of Example 1. MS (ESI) m/z 449.3 [M+H]⁺. ¹H NMR (400 MHz, DMSO-d₆) δ ppm 8.59 (brs, 1H), 7.84 (dd, J=4.80, 1.26 Hz, 1H), 7.75 (dd, J=3.54, 1.01 Hz, 1H), 7.25 (dd, J=4.80, 3.79 Hz, 1H), 7.09 (s, H), 3.36 (brs, 4H), 3.30-3.23 (m, 2H), 3.16 (brs, 4H), 1.62-1.47 (m, 4H), 1.39 (s, 9H), 1.37-1.22 (m, 4H).

### Example 3: N-(5-(Piperazin-1-yl)pentyl)-5-(thiophen-2-yl)isoxazole-3-carboxamide

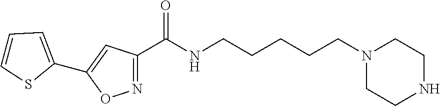 [SMILES: O=C(NCCCCCN1CCNCC1)c1cc(-c2cccs2)on1]

The title compound was prepared by using a procedure similar to that of Example 1. MS (ESI) m/z 349.1 [M+H]⁺. ¹H NMR (400 MHz, DMSO-d₆) δ ppm 8.61 (t, J=5.56 Hz, 1H), 7.84 (dd, J=5.05, 1.01 Hz, 1H), 7.75 (dd, J=3.54, 1.01 Hz, 1H), 7.25 (dd, J=5.05, 3.54 Hz, 1H), 7.09 (s, 1H), 3.27 (q, J=6.57 Hz, 2H), 3.22-3.13 (m, 4H), 2.84 (brs, 4H), 2.61 (brs, 2H), 1.55 (tt, J=13.96, 7.26 Hz, 4H), 1.41-1.26 (m, 2H).

### Example 4: N-(5-(2,5-Diazabicyclo[2.2.1]heptan-2-yl)pentyl)-5-(thiophen-2-yl)isoxazole-3-carboxamide

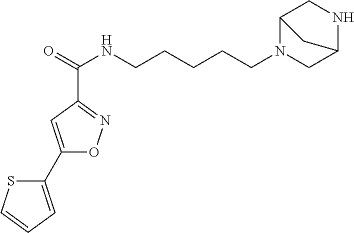 [SMILES: O=C(NCCCCCN1CC2CC1CN2)c1cc(-c2cccs2)on1]

The title compound was prepared by using a procedure similar to that of Example 1. MS (ESI) m/z 361.1 [M+H]⁺. ¹H NMR (400 MHz, DMSO-d₆) δ ppm 8.63 (t, J=5.56 Hz, 1H), 7.84 (dd, J=4.80, 1.26 Hz, 1H), 7.75 (dd, J=3.79, 1.26 Hz, 1H), 7.25 (dd, J=5.05, 3.54 Hz, 1H), 7.09 (s, 1H), 4.40 (d, J=4.55 Hz, 1H), 4.29 (brs, 1H), 3.52 (d, J=12.63 Hz, 1H), 3.39-3.22 (m, 4H), 3.15-2.91 (m, 2H), 2.25 (brs, 1H), 1.98 (d, J=11.62 Hz, 1H), 1.67-1.52 (m, 4H), 1.47-1.29 (m, 3H), 0.84 (t, J=7.33 Hz, 1H).

### Example 5: (R)—N-(5-(2-Methylpiperazin-1-yl)pentyl)-5-(thiophen-2-yl)isoxazole-3-carboxamide

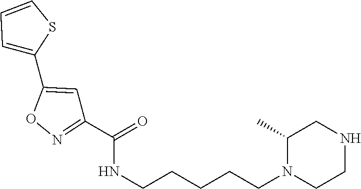 [SMILES: C[C@@H]1CNCCN1CCCCCNC(=O)c1cc(-c2cccs2)on1]

The title compound was prepared by using a procedure similar to that of Example 1. HRMS: 362.1776. ¹H NMR (400 MHz, DMSO-d₆) δ ppm 8.58 (t, J=5.05 Hz, 1H), 7.84 (dd, J=5.05, 1.01 Hz, 1H), 7.75 (dd, J=3.79, 1.26 Hz, 1H), 7.25 (dd, J=5.05, 3.54 Hz, 1H), 7.08 (s, 1H), 3.30-3.22 (m, 2H), 3.04 (dd, J=19.20, 11.12 Hz, 2H), 2.92-2.81 (m, 2H), 2.75-2.62 (m, 2H), 2.60-2.52 (m, 2H), 2.38-2.21 (m, 2H), 1.55 (quin, J=7.07 Hz, 2H), 1.48-1.37 (m, 2H), 1.36-1.24 (m, 2H), 1.01 (d, J=6.06 Hz, 3H).

### Example 6: (S)—N-(5-(3-isobutylpiperazin-1-yl)pentyl)-5-(thiophen-2-yl)isoxazole-3-carboxamide

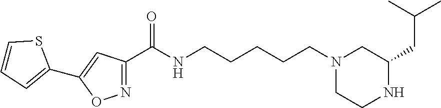 [SMILES: CC(C)C[C@H]1CN(CCCCCNC(=O)c2cc(-c3cccs3)on2)CCN1]

The title compound was prepared by using a procedure similar to that of Example 1. MS (ESI) m/z 405.3 [M+H]⁺. HRMS: 424.1933. ¹H NMR (400 MHz, DMSO-d₆) δ ppm 8.60 (t, J=5.56 Hz, 1H), 7.84 (dd, J=5.05, 1.01 Hz, 1H), 7.75 (dd, J=3.79, 1.26 Hz, 1H), 7.25 (dd, J=5.05, 4.04 Hz, 1H), 7.09 (s, 1H), 3.32-3.14 (m, 5H), 3.11-2.92 (m, 4H), 2.47-2.42 (m, 1H), 2.42-2.30 (m, 1H), 2.26-2.08 (m, 1H), 1.70 (dquin, J=13.48, 6.73, 6.73, 6.73, 6.73 Hz, 1H), 1.61-1.45 (m, 4H), 1.43-1.26 (m, 4H), 0.88 (t, J=6.32 Hz, 6H).

### Example 7: N-(5-(Pyrrolidin-1-yl)pentyl)-5-(thiophen-2-yl)isoxazole-3-carboxamide

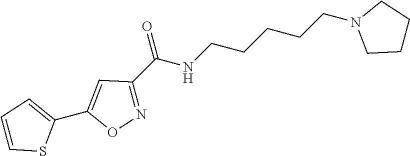 [SMILES: O=C(NCCCCCN1CCCC1)c1cc(-c2cccs2)on1]

### Step 1: Preparation of 2-(5-(Pyrrolidin-1-yl)pent-3-yn-1-yl)isoindoline-1,3-dione

To a 40 mL vial with magnetic stir bar was added 2-(but-3-yn-1-yl)isoindoline-1,3-dione (1 g, 5.02 mmol, 1 eq) followed by CuI (0.019 g, 0.100 mmol, 0.02 eq). The flask was evacuated and placed under a nitrogen atmosphere. The solids were suspended in dimethylsulfoxide (10.04 mL) and to this was added pyrrolidine (0.498 mL, 6.02 mmol, 1.2 eq) and formaldehyde (2 mL, 26.9 mmol, 5.35 eq). The reaction mixture stirred overnight at 40° C. at which time the green solution was filtered over celite and concentrated. The remaining liquid was taken in ethyl acetate and washed thrice with brine. The organic layer was dried over MgSO₄, filtered, and concentrated. The crude material was purified by silica gel chromatography eluting with 0-10% methanol/dichloromethane to give the title compound (1.42 g, 100% yield). MS (ESI) m/z 283.1 [M+H]⁺. ¹H NMR (400 MHz, CDCl₃) δ ppm 7.94-7.79 (d, 2H), 7.80-7.67 (d, 2H), 3.96-3.81 (t, 2H), 3.32 (s, 2H), 2.72-2.56 (t, 2H), 2.57-2.41 (m, 4H), 1.79-1.65 (m, 4H)

### Step 2. Preparation of 5-(Pyrrolidin-1-yl)pent-3-yn-1-amine

The title compound was prepared by using a procedure similar to that of the step 2 of Example 1 by replacing of tert-Butyl 4-(5-(1,3-dioxoisoindolin-2-yl)pentyl)-2-phenylpiperazine-1-carboxylate with 2-(5-(pyrrolidin-1-yl)pent-3-yn-1-yl)isoindoline-1,3-dione in 100% yield. MS (ESI) m/z 153.2 [M+H]⁺.

### Step 3. Preparation of N-(5-(Pyrrolidin-1-yl)pent-3-yn-1-yl)-5-(thiophen-2-yl)isoxazole-3-carboxamide

The title compound was prepared by using a procedure similar to that of the step 3 of Example 1 by replacing of tert-butyl 4-(5-aminopentyl)-2-phenylpiperazine-1-carboxylate with 5-(pyrrolidin-1-yl)pent-3-yn-1-amine. MS (ESI) m/z 330.2 [M+H]⁺. ¹H NMR (400 MHz, CD₃OD) δ ppm 7.69 (ddd, J=9.47, 4.42, 1.26 Hz, 2H), 7.22 (dd, J=5.05, 4.04 Hz, 1H), 6.93 (s, 1H), 3.53 (d, J=13.64 Hz, 3H), 3.38 (d, J=4.55 Hz, 2H), 2.70-2.60 (m, 4H), 2.58-2.47 (m, 2H), 1.79 (dt, J=6.95, 3.35 Hz, 4H).

### Step 4. Preparation of N-(5-(Pyrrolidin-1-yl)pentyl)-5-(thiophen-2-yl)isoxazole-3-carboxamide

To a 30 mL vial with magnetic stir bar was added N-(5-(pyrrolidin-1-yl)pent-3-yn-1-yl)-5-(thiophen-2-yl)isoxazole-3-carboxamide (22 mg, 0.067 mmol) and ethanol (2 mL), the vial was sparged with nitrogen and charged with palladium on carbon (14.21 mg, 0.013 mmol). The reaction mixture was placed under a nitrogen atmosphere and then sparged with hydrogen. The reaction was completed within 2 h as confirmed by LC/MS. The reaction vial was flushed with nitrogen, the reaction mixture was diluted with dichloromethane and filtered over celite. Volatiles were removed by rotary evaporator and the crude material was purified by reverse phase HPLC 15-40% acetonitrile/water 3.5 min gradient, Sunfire 30×50 mm 5 um column acetonitrile/water w/0.1% Formic Acid 75 mL/minl. 5 mL injection with three injections. MS (ESI) m/z 334.2 [M+H]⁺. ¹H NMR (400 MHz, CD₃OD) δ ppm 7.67 (ddd, J=10.11, 4.55, 1.01 Hz, 2H), 7.21 (dd, J=4.80, 3.79 Hz, 1H), 6.91 (s, 1H), 3.40 (t, J=7.07 Hz, 2H), 2.83 (brs, 4H), 2.77-2.66 (m, 2H), 1.90 (dt, J=6.69, 3.47 Hz, 4H), 1.73-1.59 (m, 4H), 1.50-1.36 (m, 2H).

### Example 8: N-(5-(4-Methylpiperazin-1-yl)pentyl)-5-(thiophen-2-yl)isoxazole-3-carboxamide

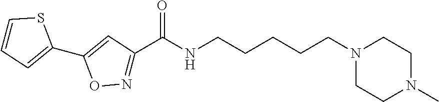 [SMILES: CN1CCN(CCCCCNC(=O)c2cc(-c3cccs3)on2)CC1]

A solution of Intermediate A (1.27 g, 6.51 mmol, 1.2), 5-(4-methylpiperazin-1-yl)pentan-1-amine hydrochloride (2.01 g, 7.16 mmol, 1.1 eq), DIEA (4.2 g, 32.53 mmol, 5.0 eq), HATU (4.95 g, 13.01 mmol, 2.0 eq) in DMF (40 mL) was stirred at 15° C. for 14 hours. The reaction mixture was purified by basic preparative HPLC (Phenomenex Gemini C18 250*50 mm*10 um, gradient: 25-55% B, (A=0.05% ammonia hydroxide/water, B=methanol), flow rate: 120 mL/min) to afford the title compound (1.926 g, 81.6% yield) as a light yellow solid. MS (ESI) m/z 363.1 [M+H]⁺. ¹H NMR (400 MHz, DMSO-d₆) δ ppm 9.09 (t, J=6.0 Hz, 1H), 8.16 (d, J=5.6 Hz, 1H), 8.08 (dd, J=4.0 Hz, 2.8 Hz, 1H), 7.56 (d, J=4.8 Hz, 1H), 7.46 (s, 1H), 3.55-3.50 (m, 2H), 2.79-2.58 (m, 12H), 2.50 (s, 3H), 1.88-1.71 (m, 4H), 1.59-1.57 (m, 2H).

### Example 9: N-(5-Morpholinopentyl)-5-(thiophen-2-yl)isoxazole-3-carboxamide

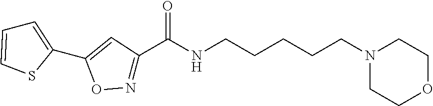 [SMILES: O=C(NCCCCCN1CCOCC1)c1cc(-c2cccs2)on1]

The title compound was prepared by using a procedure similar to that of Example 8. MS (ESI) m/z 350.1 [M+H]⁺. ¹H NMR (400 MHz, DMSO-d₆) δ ppm 8.63 (t, J=5.56 Hz, 1H), 7.84 (dd, J=5.05, 1.01 Hz, 1H), 7.75 (dd, J=3.54, 1.01 Hz, 1H), 7.25 (dd, J=5.05, 3.54 Hz, 1H), 7.09 (s, 1H), 3.94 (brs, 2H), 3.43 (brs, 4H), 3.32-3.25 (m, 2H), 3.12-3.04 (m, 4H), 1.68 (dt, J=15.66, 7.83 Hz, 2H), 1.59 (quin, J=7.20 Hz, 2H), 1.36 (quin, J=7.58 Hz, 2H).

### Example 10: 5-(4-Fluorophenyl)-N-(5-(4-methylpiperazin-1-yl)pentyl)isoxazole-3-carboxamide

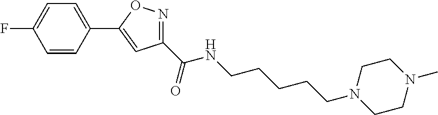 [SMILES: CN1CCN(CCCCCNC(=O)c2cc(-c3ccc(F)cc3)on2)CC1]

To a solution of 5-(4-fluorophenyl)isoxazole-3-carboxylic acid (150 mg, 0.723 mmol, 1.0 eq) in DCM (5 mL) was added (COCl)₂ (186 mg, 1.45 mmol, 2.0 eq) and DMF (1 drop). The mixture was stirred at 7-11° C. for 1 hour. The solvent was volatilized under N₂. The residue was dissolved in DCM (3 mL) and added to a solution of 5-(4-methylpiperazin-1-yl)pentan-1-amine hydrochloride (213 mg, 0.723 mmol, 1.0 eq) and Et₃N (438 mg, 4.33 mmol, 6.0 eq) in DCM (10 mL). The mixture was stirred at 7-11° C. for 16 hour. The mixture was concentrated to obtained the crude product, which was purified by preparative HPLC (Kromasil 150*25 mm*10 um, gradient: 25-55% B (A=0.05% ammonia hydroxide/water, B=MeCN), flow rate: 30 mL/min) to afford the title compound (115.5 mg, 42.6%) as a white solid. MS (ESI) m/z 375.1 [M+H]⁺. ¹H NMR (400 MHz, CD₃OD) δ ppm 7.95-7.89 (m, 2H), 7.31-7.24 (m, 2H), 7.04 (s, 1H), 3.40 (t, J=7.2 Hz, 2H), 3.00-2.00 (m, 13H), 1.68-1.64 (m, 2H), 1.61-1.51 (m, 2H), 1.45-1.36 (m, 2H).

### Example 11: N-(5-(3,8-Diazabicyclo[3.2.1]octan-3-yl)pentyl)-5-(thiophen-2-yl)isoxazole-3-carboxamide

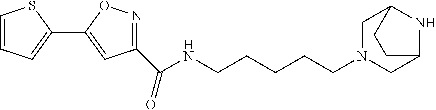 [SMILES: O=C(NCCCCCN1CC2CCC(C1)N2)c1cc(-c2cccs2)on1]

### Step 1: Preparation of tert-Butyl 3-(5-(5-(thiophen-2-yl)isoxazole-3-carboxamido)pentyl)-3,8-diazabicyclo[3.2.1]octane-8-carboxylate

NaBH(OAc)₃ (342.67 mg, 1.62 mmol, 1.5 eq) was added to a stirred solution of Intermediate B (300 mg, 1.08 mmol, 1.0 eq), tert-butyl 3,8-diazabicyclo[3.2.1]octane-8-carboxylate (343.2 mg, 1.62 mmol, 1.5 eq) and HOAc (64.73 mg, 1.08 mmol, 1.0 eq) in 1,2-dichloroethane (12 mL) at 6° C. Then the mixture was stirred at 6° C. for 14 hours. The mixture was basified with saturated NaHCO₃ aqueous solution to pH 8. The water phase was extracted with CH₂Cl₂ (3*3 mL). The combined organic layers were dried over anhydrous sodium sulfate and concentrated under reduced pressure. The residue was purified by silica gel chromatography eluting with petroleum ether/EtOAc from 20/1 to 1/1 to afford the title compound (450 mg, 81.2% yield) as a colorless oil. MS (ESI) m/z 475.2 [M+H]⁺. ¹H NMR (400 MHz, CDCl₃) δ ppm 7.56 (d, J=3.2 Hz, 1H), 7.52 (d, J=4.8 Hz, 1H), 7.18-7.16 (m, 1H), 6.84 (s, 1H), 4.19-4.10 (m, 2H), 3.50-3.45 (m, 2H), 2.64-2.62 (m, 2H), 2.33-2.29 (m, 2H), 2.25-2.18 (m, 2H), 1.85-1.82 (m, 4H), 1.65-1.61 (m, 4H), 1.48 (s, 9H), 1.44-1.42 (m, 2H).

### Step 2: Preparation of N-(5-(3,8-Diazabicyclo[3.2.1]octan-3-yl)pentyl)-5-(thiophen-2-yl)isoxazole-3-carboxamide

To a stirred solution of tert-Butyl 3-(5-(5-(thiophen-2-yl)isoxazole-3-carboxamido)pentyl)-3,8-diazabicyclo[3.2.1]octane-8-carboxylate (0.25 g, 0.5267 mmol, 1.0 eq) in CH₂Cl₂ (2 mL) was added TFA (0.744 mg, 7.8 mmol, 6.53 mmol, 12.4 eq) at 4° C. The mixture was stirred at 4° C. for 5 hours. The solvent was removed under reduced pressure. The residue was purified by basic preparative HPLC (Kromasil 150*25 mm*10 um, gradient: 22-52% B (A=0.05% ammonia hydroxide/water), B=MeCN), flow rate: 30 mL/min) to afford the title compound (34.6 mg, 35% yield) as a white solid. MS (ESI) m/z 375.1 [M+H]⁺. ¹H NMR (400 MHz, DMSO-d₆) δ ppm 8.79 (t, J=5.6 Hz, 1H), 7.88 (d, J=5.2 Hz, 1H), 7.80 (d, J=3.2 Hz, 1H), 7.27 (t, J=4.8 Hz, 1H), 7.18 (s, 1H), 3.38-3.34 (m, 3H), 3.25-3.24 (m, 2H), 2.59-2.57 (m, 2H), 2.21-2.19 (m, 2H), 2.06-2.04 (m, 2H), 1.71-1.69 (m, 2H), 1.57-1.51 (m, 4H), 1.41-1.38 (m, 2H), 1.31-1.29 (m, 2H).

### Example 12: N-(5-(8-Methyl-3,8-diazabicyclo[3.2.1]octan-3-yl)pentyl)-5-(thiophen-2-yl)isoxazole-3-carboxamide

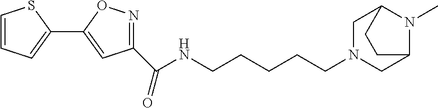 [SMILES: CN1C2CCC1CN(CCCCCNC(=O)c1cc(-c3cccs3)on1)C2]

To a solution of compound Example 11 (103.5 mg, 0.267 mmol, 1.0 eq) in MeOH (2 mL) was added paraformaldehyde (48 mg, 0.534 mmol, 2.0 eq), NaBH₃CN (67 mg, 1.1 mmol, 4.0 eq), DIEA (103.5 mg, 0.801 mmol, 3.0 eq). The mixture was stirred at 7° C. for 5 hours. The mixture was diluted with water (5 mL). The aqueous phase was extracted with CH₂Cl₂ (3*3 mL). The combined organic phase was dried over Na₂SO₄, filtered, and concentrated. The residue was purified by basic preparative HPLC (Waters Xbridge Prep OBD C18 150*30 5 u, gradient: 38-68% B (A=0.05% ammonia hydroxide/water), B=CH₃CN), flow rate: 25 mL/min) to afford the title compound (25.2 mg, 24.3% yield) as a white solid. MS (ESI) m/z 389.1 [M+H]⁺. ¹H NMR (400 MHz, DMSO-d₆) δ ppm 8.80 (t, J=5.6 Hz, 1H), 7.88 (d, J=4.8 Hz, 1H), 7.80 (d, J=3.2 Hz, 1H), 7.27 (q, J=4.0 Hz, 4.8 Hz, 1H), 7.17 (s, 1H), 3.26-3.23 (m, 2H), 2.93 (s, 2H), 2.47-2.45 (m, 2H), 2.17-2.13 (m, 2H), 2.10 (s, 3H), 2.09-2.06 (m, 2H), 1.76-1.75 (m, 2H), 1.60-1.58 (m, 2H), 1.51-1.50 (m, 2H), 1.37-1.28 (m, 4H).

### Example 13: N-(5-(3-Methyl-3,8-diazabicyclo[3.2.1]octan-8-yl)pentyl)-5-(thiophen-2-yl)isoxazole-3-carboxamide

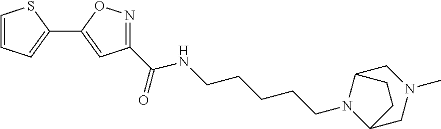 [SMILES: CN1CC2CCC(C1)N2CCCCCNC(=O)c1cc(-c2cccs2)on1]

The title compound was prepared by using a procedure similar to that of Example 11 and Example 12. MS (ESI) m/z 389.2 [M+H]⁺. ¹H NMR (400 MHz, DMSO-d₆) δ ppm 8.80 (t, J=5.2 Hz, 1H), 7.87 (dd, J=4.8 Hz, 0.8 Hz, 1H), 7.79 (dd, J=4.0 Hz, 0.8 Hz, 1H), 7.28-7.26 (m, 1H), 7.17 (s, 1H), 3.26-3.21 (m, 2H), 3.06 (brs, 2H), 2.46-2.43 (m, 2H), 2.23 (t, J=6.4 Hz, 2H), 2.10-2.05 (m, 2H), 2.07 (s, 3H), 1.75-1.72 (m, 2H), 1.65-1.57 (m, 2H), 1.56-1.47 (m, 2H), 1.44-1.36 (m, 2H), 1.35-1.25 (m, 2H).

### Example 14: N-(5-(3,5-Dimethylpiperazin-1-yl)pentyl)-5-(thiophen-2-yl)isoxazole-3-carboxamide

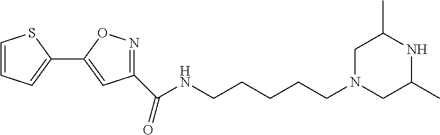 [SMILES: CC1CN(CCCCCNC(=O)c2cc(-c3cccs3)on2)CC(C)N1]

The title compound was prepared by using a procedure similar to that of Example 11. MS (ESI) m/z 377.0 [M+H]⁺. ¹H NMR (400 MHz, DMSO-d₆) δ ppm 8.80 (t, J=5.6 Hz, 1H), 7.88 (d, J=5.2 Hz, 1H), 7.80 (d, J=2.8 Hz, 1H), 7.28 (t, J=4.8 Hz, 1H), 7.18 (s, 1H), 3.27-3.24 (m, 2H), 2.73-2.66 (m, 4H), 2.22-2.18 (m, 2H), 1.55-1.51 (m, 2H), 1.43-1.36 (m, 4H), 1.29-1.28 (m, 2H), 0.91 (d, J=6.0 Hz, 6H).

### Example 15: 5-(Thiophen-2-yl)-N-(5-(3,4,5-trimethylpiperazin-1-yl)pentyl)isoxazole-3-carboxamide

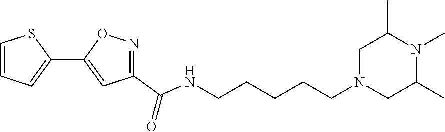 [SMILES: CC1CN(CCCCCNC(=O)c2cc(-c3cccs3)on2)CC(C)N1C]

The title compound was prepared by using a procedure similar to that of Example 12. MS (ESI) m/z 391.2 [M+H]⁺. ¹H NMR (400 MHz, DMSO-d₆) δ ppm 8.80 (t, J=5.6 Hz, 1H), 7.88 (d, J=4.4 Hz, 1H), 7.80 (d, J=3.6 Hz, 1H), 7.27 (dd, J=5.2 Hz, 4.0 Hz, 1H), 7.18 (s, 1H), 3.26-3.21 (m, 2H), 2.67-2.50 (m, 2H), 2.17-2.13 (m, 2H), 2.10-2.06 (m, 5H), 1.65-1.59 (m, 2H), 1.52-1.50 (m, 2H), 1.49-1.41 (m, 2H), 1.29-1.27 (m, 2H), 0.94 (d, J=6.0 Hz, 6H).

### Example 16: N-(5-(3-Acetamidoazetidin-1-yl)pentyl)-5-(thiophen-2-yl)isoxazole-3-carboxamide

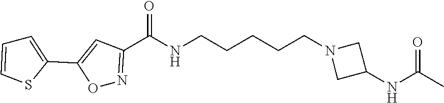 [SMILES: CC(=O)NC1CN(CCCCCNC(=O)c2cc(-c3cccs3)on2)C1]

### Step 1: Preparation of tert-Butyl (1-(5-(5-(thiophen-2-yl)isoxazole-3-carboxamido)pentyl)azetidin-3-yl)carbamate

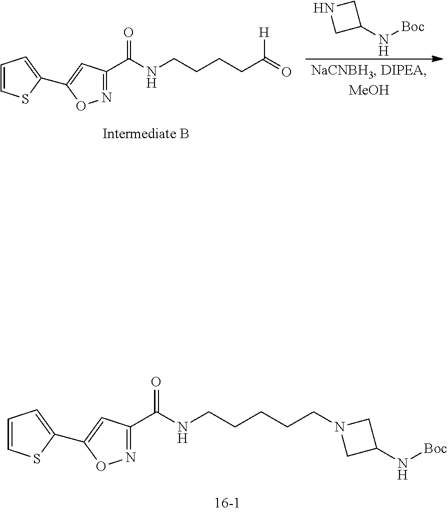 [SMILES: O=CCCCCNC(=O)c1cc(-c2cccs2)on1 ; CC(C)(C)OC(=O)NC1CNC1 ; CC(C)(C)OC(=O)NC1CN(CCCCCNC(=O)c2cc(-c3cccs3)on2)C1]

The title compound was prepared by using a procedure similar to that of step 1 of Example 11 and was obtained as a colorless oil in 100% yield. MS (ESI) m/z 435.2 [M+H]⁺.

### Step 2: Preparation of N-(5-(3-Aminoazetidin-1-yl)pentyl)-5-(thiophen-2-yl)isoxazole-3-carboxamide

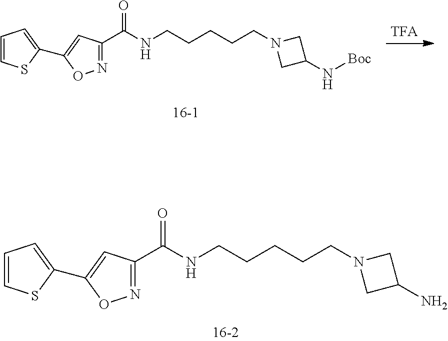 [SMILES: CC(C)(C)OC(=O)NC1CN(CCCCCNC(=O)c2cc(-c3cccs3)on2)C1 ; NC1CN(CCCCCNC(=O)c2cc(-c3cccs3)on2)C1]

The title compound was prepared by using a procedure similar to that of Example 11 and used without further purification.

### Step 3: Preparation of N-(5-(3-Acetamidoazetidin-1-yl)pentyl)-5-(thiophen-2-yl)isoxazole-3-carboxamide

To a solution of tert-butyl (1-(5-(5-(thiophen-2-yl)isoxazole-3-carboxamido)pentyl)azetidin-3-yl)carbamate (246.3 mg, 0.736 mmol, 1.0 eq) in DMF (2 mL) was added DIEA (380.7 mg, 2.95 mmol, 4.0 eq), HOAc (53.1 mg, 0.883 mmol, 1.2 eq), HATU (560 mg, 1.47 mmol, 2.0 eq) at 15° C. The mixture was stirred at 15° C. for 14 hours. The reaction was purified by basic pre-HPLC (Waters Xbridge Prep OBD C18 150*30 5 u, gradient: 55-85% B (A=0.05% ammonia hydroxide/water, B=MeOH), flow rate: 25 mL/min) to afford the title compound (118.6 mg, 42.7% yield) as a white solid. MS (ESI) m/z 377.2 [M+H]⁺. ¹H NMR (400 MHz, DMSO-d₆) δ ppm 8.79 (t, J=5.6 Hz, 1H), 8.24 (d, J=7.2 Hz, 1H), 7.86 (d, J=5.2 Hz, 1H), 7.79-7.78 (m, 1H), 7.28-7.26 (m, 1H), 7.17 (s, 1H), 4.22-4.20 (m, 2H), 3.49-3.33 (m, 2H), 3.25-3.20 (m, 2H), 2.77-2.75 (m, 2H), 2.49-2.35 (m, 2H), 1.77 (s, 3H), 1.51-1.48 (m, 2H), 1.28-1.25 (m, 4H).

### Example 17: Methyl (1-(5-(5-(thiophen-2-yl)isoxazole-3-carboxamido)pentyl)azetidin-3-yl)carbamate

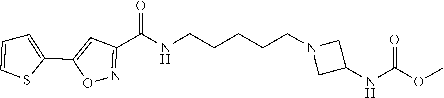 [SMILES: COC(=O)NC1CN(CCCCCNC(=O)c2cc(-c3cccs3)on2)C1]

To a solution of compound 16-2 (289 mg, 0.864 mmol, 1.0 eq) in CH₂Cl₂ (5 mL) was added Et₃N (437.2 mg, 4.32 mmol, 5.0 eq). The mixture was stirred at 15° C. for 10 min, then CDI (1.4 g, 8.64 mmol, 10.0 eq) was added. The mixture was stirred for another 4 hours. MeOH (5 mL) was added. The mixture was heated under reflux for 2 hours. The solvent was removed under reduced pressure. The residue was purified by basic preparative HPLC (Waters Xbridge Prep OBD C18 150*30 5 u, gradient: 55-85% B (A=0.05% ammonia hydroxide/water), B=MeOH), flow rate: 25 mL/min) to afford the title compound (178.4 mg, 47.2% yield) as a white solid. MS (ESI) m/z 393.1 [M+H]⁺. ¹H NMR (400 MHz, DMSO-d₆) δ ppm 8.79 (t, J=5.6 Hz, 1H), 7.86 (d, J=5.2 Hz, 1H), 7.78 (d, J=5.2 Hz, 1H), 7.58-7.57 (m, 1H), 7.28-7.25 (m, 1H), 7.16 (s, 1H), 4.02-4.00 (m, 2H), 3.49-3.41 (m, 4H), 3.22-3.20 (m, 2H), 2.70-2.68 (m, 2H), 2.33-2.29 (m, 2H), 1.50-1.47 (m, 2H), 1.27-1.23 (m, 4H).

### Example 18: N-(5-(3-(3-Methylureido)azetidin-1-yl)pentyl)-5-(thiophen-2-yl)isoxazole-3-carboxamide

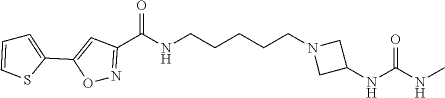 [SMILES: CNC(=O)NC1CN(CCCCCNC(=O)c2cc(-c3cccs3)on2)C1]

The title compound was prepared by using a procedure similar to that of Example 17 by replacing of methanol with methyl amine, and was obtained in 25.6% yield as a white solid. MS (ESI) m/z 392.2 [M+H]⁺. ¹H NMR (400 MHz, DMSO-d₆) δ ppm 8.80 (t, J=6.0 Hz, 1H), 7.86 (d, J=6.0 Hz, 1H), 7.78 (t, J=2.4 Hz, 1H), 7.26 (dd, J=5.2 Hz, 4.0 Hz, 1H), 7.15 (s, 1H), 6.31 (d, J=8.0 Hz, 1H), 5.69-5.67 (m, 1H), 4.12-4.06 (m, 1H), 3.30-3.25 (m, 2H), 3.24-3.21 (m, 2H), 2.65-2.52 (m, 2H), 2.51 (s, 3H), 2.33-2.31 (m, 2H), 1.51-1.47 (m, 2H), 1.26-1.22 (m, 4H).

### Example 19: N-(5-(3-(Methylsulfonamido)azetidin-1-yl)pentyl)-5-(thiophen-2-yl)isoxazole-3-carboxamide

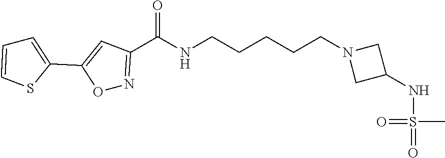 [SMILES: CS(=O)(=O)NC1CN(CCCCCNC(=O)c2cc(-c3cccs3)on2)C1]

To a solution of compound 16-2 (97 mg, 0.29 mmol, 1.0 eq) in pyridine (3 mL) was added MsCl (49.8 mg, 0.435 mmol, 1.5 eq) at 0-5° C. The mixture was allowed to warm to 25° C. and stirred for 14 hours. The mixture was quenched with saturated NaHCO₃ aqueous solution (2 mL). The solvent was removed under reduced pressure. The residue was partioned between water (5 mL) and EtOAc (5 mL). The aqueous phase was extracted with EtOAc (3*5 mL). The combined organic phase was dried over Na₂SO₄, filtered, concentrated and purified by basic preparative HPLC (Waters Xbridge Prep OBD C18 150*30 5 u, gradient: 30-60% B (A=0.05% ammonia hydroxide/water), B=CH₃CN), flow rate: 25 mL/min) to afford the title compound (17.6 mg, 14.7% yield) as a white solid. MS (ESI) m/z 413.0 [M+H]⁺. ¹H NMR (400 MHz, DMSO-d₆) δ ppm 10.72-10.69 (m, 1H), 8.85 (t, J=5.6 Hz, 1H), 8.12 (d, J=7.8 Hz, 1H), 7.87-7.78 (m, 2H), 7.28-7.18 (m, 3H), 4.46-3.90 (m, 5H), 3.27-2.90 (m, 4H), 2.96 (s, 3H), 1.50-1.47 (m, 4H), 1.32-1.29 (m, 2H).

### Example 20: 2-Hydroxyethyl (1-(5-(5-(thiophen-2-yl)isoxazole-3-carboxamido)pentyl)azetidin-3-yl)carbamate

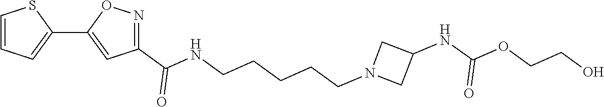 [SMILES: O=C(NC1CN(CCCCCNC(=O)c2cc(-c3cccs3)on2)C1)OCCO]

The title compound was prepared by using a procedure similar to that of Example 17 by replacing of methanol with ethane-1,2-diol, and was obtained as a white solid. MS (ESI) m/z 423.2 [M+H]⁺. ¹H NMR (400 MHz, CD₃OD) δ ppm 7.71-7.68 (m, 2H), 7.22 (t, J=4.8 Hz, 1H), 6.92 (s, 1H), 4.26-4.25 (m, 1H), 4.10-4.09 (m, 2H), 3.71-3.68 (m, 4H), 3.41-3.38 (m, 2H), 2.97-2.95 (m, 2H), 2.53-2.50 (m, 2H), 1.67-1.63 (m, 2H), 1.43-1.40 (m, 4H).

### Example 21: 2-Cyanoethyl (1-(5-(5-(thiophen-2-yl)isoxazole-3-carboxamido)pentyl)azetidin-3-yl)carbamate

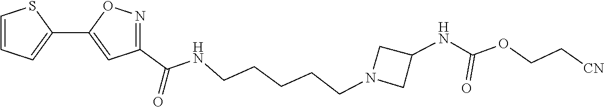 [SMILES: N#CCCOC(=O)NC1CN(CCCCCNC(=O)c2cc(-c3cccs3)on2)C1]

The title compound was prepared by using a procedure similar to that of Example 17 by replacing of methanol with 3-hydroxypropanenitrile, and was obtained as a white solid. MS (ESI) m/z 432.2 [M+H]⁺. ¹H NMR (400 MHz, CD₃OD) δ ppm 7.69-7.66 (m, 2H), 7.21 (t, J=3.6 Hz, 1H), 6.90 (s, 1H), 4.26-4.19 (m, 3H), 3.70-3.68 (m, 2H), 3.40-3.38 (m, 2H), 2.97-2.95 (m, 2H), 2.80-2.77 (m, 2H), 2.51-2.49 (m, 2H), 1.65-1.61 (m, 2H), 1.43-1.40 (m, 4H).

### Example 22: 5-(4-Fluorophenyl)-N-(5-(3-(methylsulfonamido)azetidin-1-yl)pentyl)isoxazole-3-carboxamide

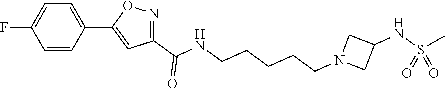 [SMILES: CS(=O)(=O)NC1CN(CCCCCNC(=O)c2cc(-c3ccc(F)cc3)on2)C1]

The title compound was prepared by using a procedure similar to that of Example 19 from Intermediate C as a white solid. MS (ESI) m/z 425.1 [M+H]⁺. ¹H NMR (400 MHz, CD₃OD) δ ppm 7.97-7.93 (m, 2H), 7.32-7.28 (m, 2H), 7.07 (s, 1H), 4.08-4.03 (m, 1H), 3.76-3.72 (m, 2H), 3.43-3.39 (m, 2H), 2.98-2.94 (m, 2H), 2.91 (s, 3H), 2.54-2.50 (m, 2H), 1.67-1.64 (m, 2H), 1.44-1.42 (m, 4H).

### Example 23: 5-(4-Fluorophenyl)-N-(5-(3-(3-methylureido)azetidin-1-yl)pentyl)isoxazole-3-carboxamide

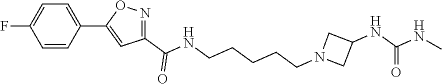 [SMILES: CNC(=O)NC1CN(CCCCCNC(=O)c2cc(-c3ccc(F)cc3)on2)C1]

The title compound was prepared by using a procedure similar to that of Example 18 from Intermediate C as an off-white solid. MS (ESI) m/z 404.2 [M+H]⁺. ¹H NMR (400 MHz, CD₃OD) δ ppm 7.97-7.93 (m, 2H), 7.32-7.28 (m, 2H), 7.07 (s, 1H), 4.34-4.32 (m, 1H), 3.68-3.66 (m, 2H), 3.43-3.39 (m, 2H), 2.93-2.91 (m, 2H), 2.68 (s, 3H), 2.51-2.47 (m, 2H), 1.67-1.64 (m, 2H), 1.44-1.42 (m, 4H).

### Example 24: Methyl 4-(5-(5-(thiophen-2-yl)isoxazole-3-carboxamido)pentyl)piperazine-1-carboxylate

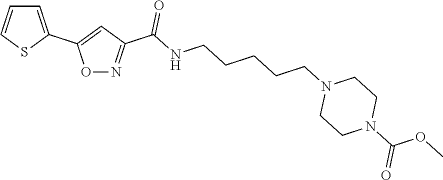 [SMILES: COC(=O)N1CCN(CCCCCNC(=O)c2cc(-c3cccs3)on2)CC1]

The title compound was prepared by using a procedure similar to that of Example 11 and Example 17 by replacing of 5-(4-methylpiperazin-1-yl)pentan-1-amine hydrochloride with tert-butyl piperazine-1-carboxylate, and was obtained as a white solid. MS (ESI) m/z 407.1 [M+H]⁺. ¹H NMR (400 MHz, DMSO-d₆+2 drop D₂O) δ ppm 8.80 (t, J=5.6 Hz, 1H), 7.87 (d, J=4.8 Hz, 1H), 7.79 (d, J=2.8 Hz, 1H), 7.27 (t, J=4.0 Hz, 1H), 7.17 (s, 1H), 3.57 (s, 3H), 3.31 (s, 4H), 3.23 (q, J=6.4 Hz, 2H), 2.28-2.23 (m, 6H), 1.54-1.48 (m, 2H), 1.47-1.40 (m, 2H), 1.32-1.27 (m, 2H).

### Example 25: N-(5-(4-(Methylcarbamoyl)piperazin-1-yl)pentyl)-5-(thiophen-2-yl)isoxazole-3-carboxamide

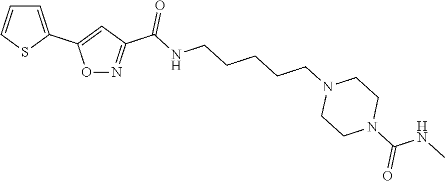 [SMILES: CNC(=O)N1CCN(CCCCCNC(=O)c2cc(-c3cccs3)on2)CC1]

The title compound was prepared by using a procedure similar to that of Example 11 and Example 18 by replacing of 5-(4-methylpiperazin-1-yl)pentan-1-amine hydrochloride with tert-butyl piperazine-1-carboxylate, and was obtained as a white solid. MS (ESI) m/z 406.1 [M+H]⁺. ¹H NMR (400 MHz, DMSO-d₆) δ ppm 8.80 (t, J=6.0 Hz, 1H), 7.87 (dd, J=4.8, 0.8 Hz, 1H), 7.79 (dd, J=4.0, 1.2 Hz, 1H), 7.26 (dd, J=4.8, 3.6 Hz, 1H), 7.17 (s, 1H), 6.38 (d, J=4.4 Hz, 1H), 3.31-3.26 (m, 6H), 2.54 (d, J=4.4 Hz, 3H), 2.27-2.22 (m, 6H), 1.56-1.48 (m, 2H), 1.47-1.40 (m, 2H), 1.32-1.24 (m, 2H).

### Example 26: N-(5-(3-Oxopyrrolidin-1-yl)pentyl)-5-(thiophen-2-yl)isoxazole-3-carboxamide

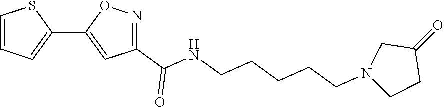 [SMILES: O=C1CCN(CCCCCNC(=O)c2cc(-c3cccs3)on2)C1]

### Step 1: Preparation of N-(5-Bromopentyl)-5-(thiophen-2-yl)isoxazole-3-carboxamide

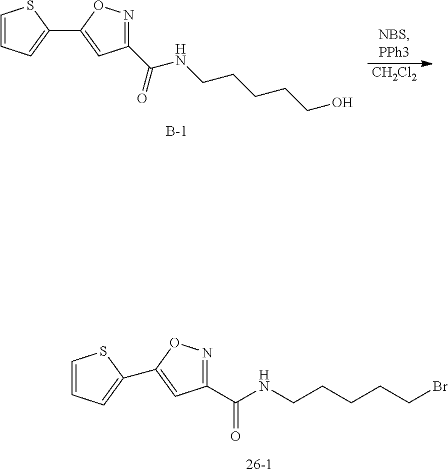

To a mixture of compound B-1 (1.0 g, 3.57 mmol, 1.0 eq) in anhydrous CH₂Cl₂ (50 mL) was added PPh₃ (1.12 g, 4.28 mmol, 1.2 eq) and NBS (716.8 mg, 4.28 mmol, 1.2 eq) at 0-5° C. under nitrogen atmosphere. The mixture was allowed to warm to 25° C. and stirred for 14 hours. The reaction mixture was poured into saturated NaHCO₃ aqueous solution (50 mL). The aqueous layer was extracted with CH₂Cl₂ (3*20 mL). The combined organic phase was dried over Na₂SO₄, filtered, concentrated and purified by silica gel chromatography eluting with petroleum ether/EtOAc from 20/1 to 5/1 to afford the title compound (0.64 g, 52.4% yield) as a light yellow oil. ¹H NMR (400 MHz, CDCl₃) δ ppm 7.55 (d, J=3.6 Hz, 1H), 7.50 (d, J=5.2 Hz, 1H), 7.14 (dd, J=5.2 Hz, 3.6 Hz, 1H), 6.87-6.86 (m, 1H), 6.82 (s, 1H), 3.49-3.40 (m, 4H), 1.93-1.89 (m, 2H), 1.68-1.64 (m, 2H), 1.58-1.54 (m, 2H).

### Step 2: Preparation of N-(5-(3-Oxopyrrolidin-1-yl)pentyl)-5-(thiophen-2-yl)isoxazole-3-carboxamide

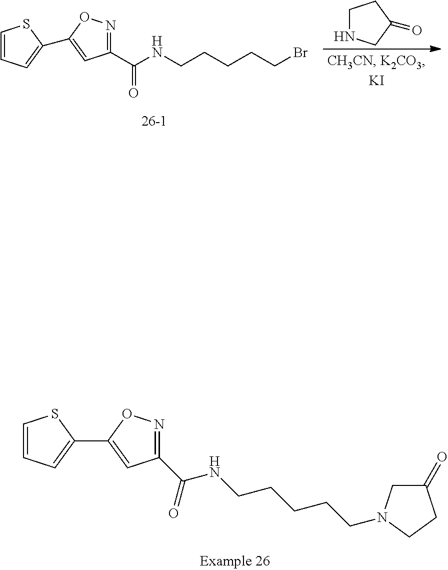 [SMILES: O=C(NCCCCCBr)c1cc(-c2cccs2)on1 ; O=C1CCNC1 ; O=C1CCN(CCCCCNC(=O)c2cc(-c3cccs3)on2)C1]

To a solution of compound 26-1 (0.1 g, 0.291 mmol, 1.0 eq) in CH₃CN (2 mL) was added KI (58 mg, 0.349 mmol, 1.2 eq), pyrrolidin-3-one hydrochloride (70.8 mg, 0.582 mmol, 2.0 eq), K₂CO₃ (120.8 mg, 0.874 mmol, 3.0 eq). The mixture was stirred at 25° C. for 14 hours. To the reaction mixture was added pyrrolidin-3-one hydrochloride (71 mg, 0.582 mmol, 2.0 eq) and K₂CO₃ (121 mg, 0.874 mmol, 3.0 eq). The mixture was stirred at 25° C. for 14 hours. Then another portion of pyrrolidin-3-one hydrochloride (71 mg, 0.582 mmol, 2.0 eq) and K₂CO₃ (121 mg, 0.874 mmol, 3.0 eq) was added and the mixture was stirred at 25° C. for 62 hours. The mixture was diluted with water (5 mL), extracted with EtOAc (3*10 mL). The combined organic phase was dried over Na₂SO₄, filtered, concentrated and purified by basic preparative HPLC (Waters Xbridge Prep OBD C18 150*30 5 u, gradient: 50-80% B (A=0.05% ammonia hydroxide/water, B=MeOH), flow rate: 25 mL/min) to afford the title compound (9.7 mg, 9.58% yield) as a light yellow solid. MS (ESI) m/z 347.9 [M+H]⁺. ¹H NMR (400 MHz, DMSO-d₆) δ ppm 8.81 (s, 1H), 7.88 (d, J=4.8 Hz, 0.8 Hz, 1H), 7.80 (d, J=4.4 Hz, 1.2 Hz, 1H), 7.28 (t, J=4.8 Hz, 3.6 Hz, 1H), 7.18 (s, 1H), 3.34-3.26 (m, 2H), 2.86 (s, 2H), 2.83-2.80 (m, 2H), 2.49-2.48 (m, 2H), 2.33-2.29 (m, 2H), 1.55-1.46 (m, 4H), 1.35-1.34 (m, 2H).

### Example 27: N-(5-(3-Carbamoylazetidin-1-yl)pentyl)-5-(thiophen-2-yl)isoxazole-3-carboxamide

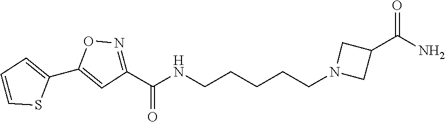 [SMILES: NC(=O)C1CN(CCCCCNC(=O)c2cc(-c3cccs3)on2)C1]

To a mixture of Intermediate B (283.1 mg, 2.07 mmol, 1.0 eq) in MeOH (10 mL) was added Et₃N (230.7 mg, 2.28 mmol, 1.1 eq) at 8° C. The mixture was stirred at 8° C. for 5 min, then azetidine-3-carboxamide hydrochloride (0.577 g, 2.07 mmol, 1.0 eq) was added in one portion. The mixture was stirred at 8° C. for 1.5 hours. To the mixture was added NaBH₃CN (260.5 mg, 4.15 mmol, 2.0 eq) at 8° C. The mixture was stirred at 8° C. for 14 hours. The reaction mixture was quenched with water (20 mL), and MeOH was removed under reduced pressure. The aqueous phase was extracted with EtOAc (3*10 mL). The combined organic phase was dried over Na₂SO₄, filtered and concentrated under reduced pressure. The residue was purified by acidic preparative HPLC (Phenomenex luna C18 250*50 mm*10 um, gradient: 10-40% B (A=0.1% TFA/water), B=MeCN), flow rate: 120 mL/min). The obtained fraction was basified with saturated NaHCO₃ aqueous solution to pH 8, and the aqueous phase was extracted with EtOAc (3*200 mL). The combined organic phase was dried over Na₂SO₄, concentrated. The residue was lyophilized to afford the title compound (350 mg, 22.4% yield) as a white solid. MS (ESI) m/z 363.0 [M+H]⁺. ¹H NMR (400 MHz, DMSO-d₆) δ ppm 8.79 (t, J=5.6 Hz, 1H), 7.87 (d, J=4.0 Hz, 1H), 7.79 (d, J=2.8 Hz, 1H), 7.26 (t, J=4.0 Hz, 1H), 7.16 (s, 1H), 6.82 (s, 1H), 3.30-3.21 (m, 4H), 3.02-2.98 (m, 3H), 2.32-2.28 (m, 2H), 1.49-1.47 (m, 2H), 1.28-1.23 (m, 4H).

The following compounds, as identified in Table 1, were prepared using the general procedures as well as the procedures from the examples described above with the appropriate starting materials and reagents.

TABLE 1

| Example | | |
|---|---|---|
| No. | Structure | MS/NMR |
| 28 | 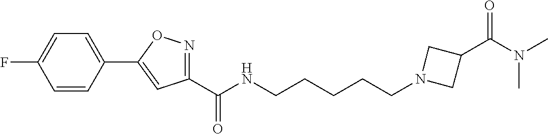 [SMILES: CN(C)C(=O)C1CN(CCCCCNC(=O)c2cc(-c3ccc(F)cc3)on2)C1] | MS (ESI) m/z 403.1 [M + H]⁺. ¹H NMR (400 MHz, CD₃OD) δ ppm 7.97-7.89 (m, 2H), 7.28 (t, J = 8.8 Hz, 2H), 7.05 (s, 1H), 3.66-3.55 (m, 3H), 3.39 (t, J = 7.2 Hz, 2H), 3.30-3.24 (m, 2H), 2.92 (s, 6H), 2.54-2.47 (m, 2H), 1.69-1.59 (m, 2H), 1.48-1.35 (m, 4H). |
| 29 | 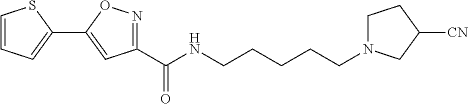 [SMILES: N#CC1CCN(CCCCCNC(=O)c2cc(-c3cccs3)on2)C1] | MS (ESI) m/z 359.1 [M + H]⁺. ¹H NMR (400 MHz, DMSO-d₆) δ ppm 8.80 (t, J = 6.0 Hz, 1H), 7.87 (d, J = 6.0 Hz, 1H), 7.79 (d, J = 3.6 Hz, 1H), 7.26 (dd, J = 4.8 Hz, 4.0 Hz, 1H), 7.17 (s, 1H), 3.35-3.21 (m, 3H), 2.66-2.52 (m, 3H), 2.79-2.37 (m, 3H), 2.40-2.20 (m, 1H), 1.90-1.60 (m, 1H), 1.57-1.43 (m, 4H), 1.32-1.30 (m, 2H). |
| 30 | 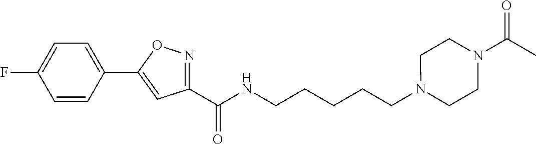 [SMILES: CC(=O)N1CCN(CCCCCNC(=O)c2cc(-c3ccc(F)cc3)on2)CC1] | MS (ESI) m/z 403.1 [M + H]⁺. ¹H NMR (400 MHz, CD₃OD) δ ppm 7.96-7.89 (m, 2H), 7.32-7.24 (m, 2H), 7.05 (s, 1H), 3.67-3.52 (m, 4H), 3.41 (t, J = 7.2 Hz, 2H), 2.63-2.40 (m, 6H), 2.09 (s, 3H), 1.74-1.54 (m, 4H), 1.48-1.38 (m, 2H). |
| 31 | 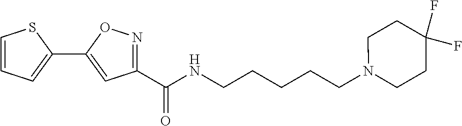 [SMILES: O=C(NCCCCCN1CCC(F)(F)CC1)c1cc(-c2cccs2)on1] | MS (ESI) m/z 384.1 [M + H]⁺. ¹HNMR (400 MHz, DMSO-d₆) δ ppm 8.80 (t, J = 6.0 Hz, 1H), 7.86 (dd, J = 4.8 Hz, 0.8 Hz, 1H), 7.79 (dd, J = 3.6 Hz, 0.8 Hz, 1H), 7.26 (dd, J = 4.4 Hz, 3.6 Hz, 1H), 7.17 (s, 1H), 5.24-5.07 (m, 1H), 3.27-3.21 (m, 2H), 2.45-2.43 (m, 4H), 2.31 (t, J = 7.2 Hz, 2H), 1.96-1.86 (m, 4H), 1.57-1.48 (m, 2H), 1.46-1.40 (m, 2H), 1.33-1.22 (m, 2H). ¹⁹FNMR (400 MHz, DMSO-d₆) δ ppm −95.84. |
| 32 | 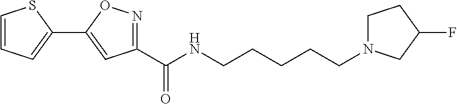 [SMILES: O=C(NCCCCCN1CCC(F)C1)c1cc(-c2cccs2)on1] | MS (ESI) m/z 352.0 [M + H]⁺. ¹HNMR (400 MHz, DMSO-d₆) δ ppm 8.80 (t, J = 5.2 Hz, 1H), 7.87 (dd, J = 4.8 Hz, 0.4 Hz ,1H), 7.79 (dd, J = 3.6 Hz, 0.8 Hz, 1H), 7.26 (dd, J = 4.8 Hz, 3.6 Hz, 1H), 7.17 (s, 1H), 5.24-5.07 (m, 1H), 3.27-3.21 (m, 2H), 2.80-2.66 (m, 2H), 2.59-2.54 (m, 1H), 2.37 (t, J = 7.2 Hz, 2H), 2.26-2.20 (m, 1H), 2.17-2.01 (m, 1H), 1.90-1.75 (m, 1H), 1.57-1.40 (m, 4H), 1.56-1.46 (m, 3H), 1.34-1.26 (m, 2H). ¹⁹FNMR (400 MHz, DMSO-d₆) δ ppm −166.16 |
| 33 | 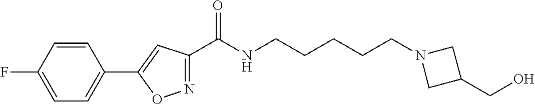 [SMILES: O=C(NCCCCCN1CC(CO)C1)c1cc(-c2ccc(F)cc2)on1] | MS (ESI) m/z 362.0 [M + H]⁺. ¹H NMR (400 MHz, CD₃OD) δ ppm 7.96-7.89 (m, 2H), 7.31-7.24 (m, 2H), 7.05 (s, 1H), 3.63 (d, J = 6.4 Hz, 2H), 3.50 (t, J = 8.4 Hz, 2H), 3.39 (t, J = 6.8 Hz, 2H), 3.18-3.11 (m, 2H), 2.76-2.64 (m, 1H), 2.61-2.54 (m, 2H), 1.71-1.58 (m, 2H), 1.50-1.35 (m, 4H). |
| 34 | 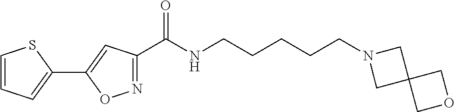 [SMILES: O=C(NCCCCCN1CC2(COC2)C1)c1cc(-c2cccs2)on1] | MS (ESI) m/z 362.0 [M + H]⁺. ¹H NMR (400 MHz, CD₃OD) δ ppm 7.71-7.67 (m, 2H), 7.29-7.18 (m, 1H), 6.92 (s, 1H), 4.74 (s, 4H), 3.43 (s, 4H), 3.40 (t, J = 6.8 Hz, 2H), 2.46 (t, J = 6.8 Hz, 2H), 1.73-1.57 (m, 2H), 1.50-1.33 (m, 4H). |
| 35 | 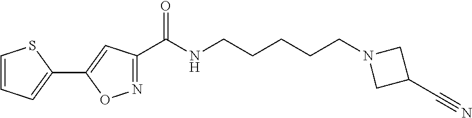 [SMILES: N#CC1CN(CCCCCNC(=O)c2cc(-c3cccs3)on2)C1] | MS (ESI) m/z 344.9 [M + H]⁺. ¹H NMR (400 MHz, CD₃OD) δ ppm 7.77-7.63 (m, 2H), 7.22 (t, J = 4.0 Hz, 1H), 6.92 (s, 1H), 3.64-3.51 (m, 2H), 3.47-3.35 (m, 5H), 2.49 (brs, 2H), 1.72-1.56 (m, 2H), 1.41 (brs, 4H). |
| 36 | 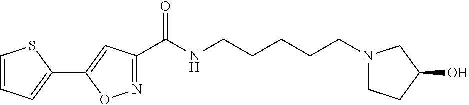 [SMILES: O=C(NCCCCCN1CC[C@H](O)C1)c1cc(-c2cccs2)on1] | MS (ESI) m/z 349.9 [M + H]⁺. ¹H NMR (400 MHz, CD₃OD) δ ppm 7.71-7.67 (m, 2H), 7.23 (dd, J = 5.2, 4.0 Hz, 1H), 6.93 (s, 1H), 4.40-4.35 (m, 1H), 3.41 (t, J = 7.2 Hz, 2H), 2.90-2.87 (m, 1H), 2.82-2.73 (m, 1H), 2.69-2.48 (m, 4H), 2.21-2.09 (m, 1H), 1.80-1.56 (m, 5H), 1.49-1.38 (m, 2H). |
| 37 | 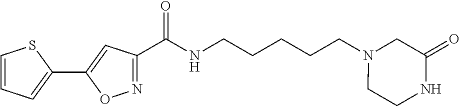 [SMILES: O=C1CN(CCCCCNC(=O)c2cc(-c3cccs3)on2)CCN1] | MS (ESI) m/z 362.9 [M + H]⁺. ¹H NMR (400 MHz, CD₃OD) δ ppm 7.71-7.67 (m, 2H), 7.22 (t, J = 4.4 Hz, 1H), 6.92 (s, 1H), 3.42 (t, J = 6.8 Hz, 2H), 3.37-3.33 (m, 2H), 3.11 (s, 2H), 2.70 (t, J = 5.6 Hz, 2H), 2.52-2.44 (m, 2H), 1.76-1.55 (m, 4H), 1.50-1.39 (m, 2H). |
| 38 | 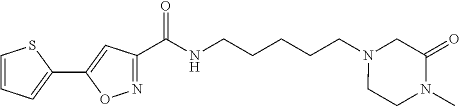 [SMILES: CN1CCN(CCCCCNC(=O)c2cc(-c3cccs3)on2)CC1=O] | MS (ESI) m/z 376.9 [M + H]⁺. ¹H NMR (400 MHz, CD₃OD) δ ppm 7.71-7.67 (m, 2H), 7.28-7.18 (m, 1H), 6.92 (s, 1H), 3.46-3.35 (m, 4H), 3.12 (s, 2H), 2.99-2.92 (m, 3H), 2.74 (t, J = 5.6 Hz, 2H), 2.51-2.40 (m, 2H), 1.74-1.54 (m, 4H), 1.51-1.38 (m, 2H). |
| 39 | 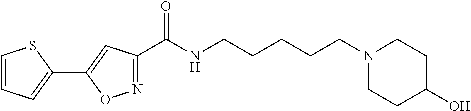 [SMILES: O=C(NCCCCCN1CCC(O)CC1)c1cc(-c2cccs2)on1] | MS (ESI) m/z 363.9 [M + H]⁺. ¹H NMR (400 MHz, CD₃OD) δ ppm7.71-7.67 (m, 2H), 7.23 (dd, J = 5.2, 4.0 Hz, 1H), 6.92 (s, 1H), 3.65 (brs, 1H), 3.41 (t, J = 6.8 Hz, 2H), 2.85 (brs, 2H), 2.40 (t, J = 8.0 Hz, 2H), 2.21 (brs, 2H), 1.92-1.86 (mz, 2H), 1.74-1.51 (m, 6H), 1.44-1.38 (m, 2H). |
| 40 | 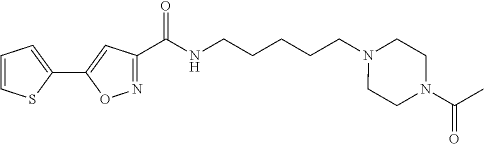 [SMILES: CC(=O)N1CCN(CCCCCNC(=O)c2cc(-c3cccs3)on2)CC1] | MS (ESI) m/z 391.0 [M + H]⁺. ¹H NMR (400 MHz, CD₃OD) δ ppm 7.70-7.63 (m, 2H), 7.23-7.17 (m, 1H), 6.90 (s, 1H), 3.63-3.48 (m, 4H), 3.39 (t, J = 7.2 Hz, 2H), 2.53-2.35 (m, 6H), 2.08 (s, 3H), 1.71-1.54 (m, 4H), 1.46-1.37 (m, 2H). |
| 41 | 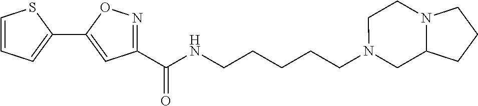 [SMILES: O=C(NCCCCCN1CCN2CCCC2C1)c1cc(-c2cccs2)on1] | MS (ESI) m/z 389.1 [M + H]⁺. ¹H NMR (400 MHz, CD₃OD) δ ppm 7.70-7.63 (m, 2H), 7.20 (t, J = 4.0 Hz, 1H), 6.90 (s, 1H), 3.39 (t, J = 7.2 Hz, 2H), 3.09-2.97 (m, 3H), 2.89 (d, J = 10.8 Hz, 1H), 2.50-2.37 (m, 2H), 2.34-2.08 (m, 4H), 1.91-1.72 (m, 4H), 1.70-1.53 (m, 4H), 1.45-1.33 (m, 3H). |
| 42 | 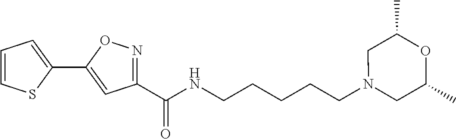 [SMILES: C[C@@H]1CN(CCCCCNC(=O)c2cc(-c3cccs3)on2)C[C@H](C)O1] | MS (ESI) m/z 378.1 [M + H]⁺. ¹H NMR (400 MHz, CD₃OD) δ ppm 7.74-7.66 (m, 2H), 7.26-7.20 (m, 1H), 6.93 (s, 1H), 3.75-3.63 (m, 2H), 3.41 (t, J = 6.8 Hz, 2H), 2.83 (d, J = 11.2 Hz, 2H), 2.43-2.33 (m, 2H), 1.78-1.56 (m, 6H), 1.50-1.38 (m, 2H), 1.15 (s, 3H), 1.13 (s, 3H). |
| 43 | 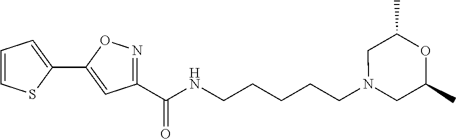 [SMILES: C[C@H]1CN(CCCCCNC(=O)c2cc(-c3cccs3)on2)C[C@H](C)O1] | MS (ESI) m/z 378.0 [M + H]⁺. ¹H NMR (400 MHz, D₂O) δ ppm 7.64 (d, J = 4.0 Hz, 2H), 7.18-7.16 (m, 1H), 6.86 (d, J = 5.2 Hz, 1H), 4.38-4.32 (m, 1H), 4.23-4.11 (m, 1H), 3.50-3.32 (m, 4H), 3.20-3.06 (m, 3H), 2.72 (t, J = 12.0 Hz, 1H), 1.83-1.56 (m, 4H), 1.46-1.31 (m, 5H), 1.21-1.09 (m, 3H). |
| 44 | 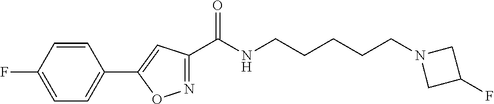 [SMILES: O=C(NCCCCCN1CC(F)C1)c1cc(-c2ccc(F)cc2)on1] | MS (ESI) m/z 350.2 [M + H]⁺. ¹H NMR (400 MHz, CD₃OD) δ ppm 7.95-7.90 (m, 2H), 7.31-7.25 (m, 2H), 7.05 (s, 1H), 5.22-5.01 (m, 1H), 3.68-3.58 (m, 2H), 3.39 (t, J = 7.2 Hz, 2H), 3.29-3.24 (m, 1H), 3.23-3.19 (m, 1H), 2.54 (t, J = 6.8 Hz, 2H), 1.67-1.61 (m, 2H), 1.48-1.32 (m, 4H). |
| 45 | 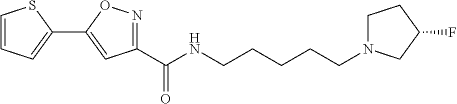 [SMILES: O=C(NCCCCCN1CC[C@H](F)C1)c1cc(-c2cccs2)on1] | MS (ESI) m/z 352.0 [M + H]⁺. ¹HNMR (400 MHz, DMSO-d₆) δ ppm 8.80 (t, J = 6.0 Hz, 1H), 7.86 (d, J = 3.6 Hz, 1H), 7.79 (d, J = 3.6 Hz, 1H), 7.26 (t, J = 4.0 Hz, 1H), 7.17 (s, 1H), 5.24-5.07 (m, 1H), 3.27-3.21 (m,H), 2.81-2.71 (m, 2H), 2.59-2.55 (m, 1H), 2.37 (t, J = 7.2 Hz, 2H), 2.27-2.21 (m, 1H), 2.17-2.01 (m, 1H), 1.90-1.76 (m, 1H), 1.56-1.49 (m, 2H), 1.48-1.40, (m, 2H), 1.34-1.26 (m, 2H). ¹⁹FNMR (400 MHz, DMSO-d₆) 0 ppm −166.22 |
| 46 | 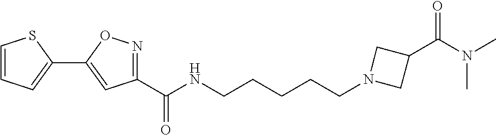 [SMILES: CN(C)C(=O)C1CN(CCCCCNC(=O)c2cc(-c3cccs3)on2)C1] | MS (ESI) m/z 391.1 [M + H]⁺. ¹H NMR: (400 MHz, DMSO-d₆) (t = 80° C.) 5 ppm 10.80 (brs, 1H), 8.52 (s, 1H), 7.84-7.83 (m, 1H), 7.74 (d, J = 3.6 Hz, 1H), 7.26 (t, J = 4.0 Hz, 1H), 7.07 (s, 1H), 4.27-4.19 (m, 4H), 3.99-3.80 (m, 1H), 3.30-3.16 (m, 2H), 3.10-3.00 (m, 2H), 2.87 (s, 6H), 1.60-1.55 (m, 4H), 1.39-1.37 (m, 2H). |
| 47 | 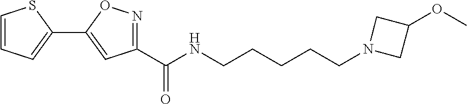 [SMILES: COC1CN(CCCCCNC(=O)c2cc(-c3cccs3)on2)C1] | MS (ESI) m/z 349.9 [M + H]⁺. ¹H NMR (400 MHz, CD₃OD) δ ppm 7.73-7.62 (m, 2H), 7.21 (dd, J = 4.0, 4.4 Hz, 1H), 6.92 (s, 1H), 4.55-4.46 (m, 1H), 4.35-4.11 (m, 3H), 3.96-3.87 (m, 1H), 3.41 (t, J = 6.8 Hz, 2H), 3.35 (d, J = 8.0 Hz, 3H), 3.28-3.18 (m, 2H), 1.72-1.59 (m, 4H), 1.49-1.40 (m, 2H). |
| 48 | 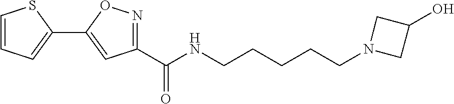 [SMILES: O=C(NCCCCCN1CC(O)C1)c1cc(-c2cccs2)on1] | MS (ESI) m/z 336.0 [M + H]⁺. ¹H NMR: (400 MHz, DMSO-d₆) δ ppm 8.80 (t, J = 6.0 Hz, 1H), 7.87 (d, J = 5.2 Hz, 1H), 7.79 (d, J = 3.2 Hz, 1H), 7.27 (t, J = 4.8 Hz, 1H), 7.17 (s, 1H), 5.21 (d, J = 6.4 Hz, 1H), 4.14-4.10 (m, 1H), 3.47-3.44 (m, 2H), 3.23-3.19 (m, 2H), 2.60-2.56 (m, 2H), 2.33-2.30 (m, 2H), 1.51-1.47 (m, 2H), 1.31-1.21 (m, 4H). |
| 49 | 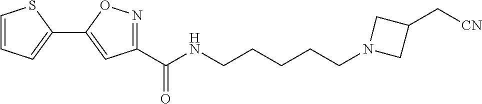 [SMILES: N#CCC1CN(CCCCCNC(=O)c2cc(-c3cccs3)on2)C1] | MS (ESI) m/z 359.1 [M + H]⁺. ¹H NMR: (400 MHz, DMSO-d₆) δ ppm 8.84 (t, J = 6.0 Hz, 1H), 7.87 (d, J = 6.0 Hz, 1H), 7.79 (d, J = 4.4 Hz, 1H), 7.27 (dd, J = 5.2 Hz, 3.6 Hz, 1H), 7.18 (s, 1H), 6.54 (s, 1H), 4.17-4.09 (m, 2H), 3.88-3.79 (m, 2H), 3.26-3.22 (m, 2H), 3.18-3.07 (m, 4H), 2.90-2.80 (m, 1H), 1.54-1.47 (m, 4H), 1.32-1.30 (m, 2H). |
| 50 | 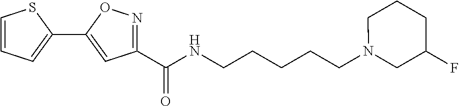 [SMILES: O=C(NCCCCCN1CCCC(F)C1)c1cc(-c2cccs2)on1] | MS (ESI) m/z 366.1 [M + H]⁺. ¹HNMR (400 MHz, DMSO-d₆) δ ppm 8.80 (t, J = 5.6 Hz, 1H), 7.87 (dd, J = 4.8 Hz, 0.8 Hz ,1H), 7.79 (dd, J = 3.6 Hz, 0.8 Hz, 1H), 7.27 (dd, J = 4.4 Hz, 3.6 Hz, 1H), 7.17 (s, 1H), 4.67-4.49 (m, 1H), 3.26-3.21 (m, 2H), 2.71-2.64 (m, 1H), 2.40-2.36 (m, 1H), 2.29-2.22 (m, 3H), 2.17-2.12 (m, 1H), 1.86-1.76 (m, 1H), 1.71-1.63 (m, 1H), 1.56-1.46 (m, 3H), 1.45-1.39 (m, 3H), 1.31-1.23 (m, 2H). ¹⁹FNMR (400 MHz, DMSO-d₆) δ ppm −178.26 |
| 51 | 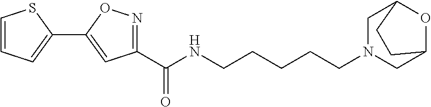 [SMILES: O=C(NCCCCCN1CC2CCC(C1)O2)c1cc(-c2cccs2)on1] | MS (ESI) m/z 376.0 [M + H]⁺. ¹H NMR (400 MHz, DMSO-d₆) δ ppm 8.79 (t, J = 5.6 Hz, 1H), 7.87 (d, J = 4.8 Hz, 1H), 7.79 (d, J = 3.2 Hz, 1H), 7.27 (d, J = 4.4 Hz, 1H), 7.17 (s, 1H), 4.16 (s, 2H), 3.26-3.21 (m, 2H), 2.53 (s, 2H), 2.20 (t, J = 6.4 Hz, 2H), 2.07-2.04 (m, 2H), 1.79-1.74 (m, 2H), 1.65-1.63 (m, 2H), 1.56-1.48 (m, 2H), 1.42-1.35 (m, 2H), 1.32-1.27 (m, 2H). |
| 52 | 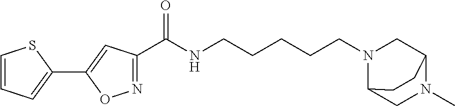 [SMILES: CN1CC2CCC1CN2CCCCCNC(=O)c1cc(-c2cccs2)on1] | MS (ESI) m/z 389.1 [M + H]⁺. ¹H NMR (400 MHz, DMSO-d₆) δ ppm 8.80 (t, J = 6.0 Hz, 1H), 7.86 (dd, J = 4.8, 0.8 Hz 1H), 7.78 (dd, J = 3.6, 1.2 Hz, 1H), 7.28-7.23 (m, 1H), 7.17 (s, 1H), 3.22 (m, 2H), 2.81-2.74 (m, 2H), 2.59-2.55 (m, 2H), 2.48-2.44 (m, 1H), 2.43-2.30 (m, 3H), 2.21 (s, 3H), 1.81-1.72 (m, 2H), 1.55-1.26 (m, 8H). |
| 53 | 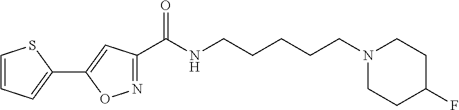 [SMILES: O=C(NCCCCCN1CCC(F)CC1)c1cc(-c2cccs2)on1] | MS (ESI) m/z 366.0 [M + H]⁺. ¹H NMR (400 MHz, CDCl₃) δ ppm 7.54 (dd, J = 3.6, 0.8 Hz, 1H), 7.48 (dd, J = 5.2, 1.2 Hz, 1H), 7.17-7.11 (m, 1H), 6.88 (br, 1H), 6.81 (s, 1H), 4.74-4.70 (m, 0.5H), 4.62-4.57 (m, 0.5H), 3.48-3.43 (m, 2H), 2.63-2.51 (m, 2H), 2.43-2.23 (m, 4H), 1.94-1.84 (m, 4H), 1.70-1.59 (m, 2H), 1.58-1.48 (m, 2H), 1.45-1.32 (m, 2H). |
| 54 | 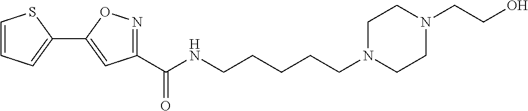 [SMILES: O=C(NCCCCCN1CCN(CCO)CC1)c1cc(-c2cccs2)on1] | MS (ESI) m/z 393.1 [M + H]⁺. ¹H NMR (400 MHz, DMSO-d₆ + 1 drop D₂O) δ ppm 8.81-8.79 (m, 1H), 7.84 (d, J = 4.8 Hz, 1H), 7.77 (d, J = 3.2 Hz, 1H), 7.25 (t, J = 4.0 Hz, 1H), 7.13 (s, 1H), 3.53-3.47 (m, 2H), 3.44 (t, J = 6.4 Hz, 2H), 3.22 (t, J = 6.8 Hz, 2H), 2.41-2.31 (m, 8H), 2.20 (t, J = 7.2 Hz, 2H), 1.54-1.47 (m, 2H), 1.47-1.36 (m, 2H), 1.29-1.21 (m, 2H). |
| 55 | 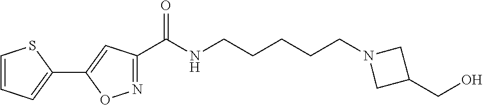 [SMILES: O=C(NCCCCCN1CC(CO)C1)c1cc(-c2cccs2)on1] | MS (ESI) m/z 349.9 [M + H]⁺. ¹H NMR (400 MHz, CD₃OD) δ ppm 7.72-7.63 (m, 2H), 7.24-7.18 (m, 1H), 6.91 (s, 1H), 3.63 (d, J = 6.0 Hz, 2H), 3.57-3.49 (m, 2H), 3.39 (t, J = 7.2 Hz, 2H), 3.23-3.15 (m, 2H), 2.78-2.66 (m, 1H), 2.64-2.57 (m, 2H), 1.69-1.57 (m, 2H), 1.50-1.35 (m, 4H). |
| 56 | 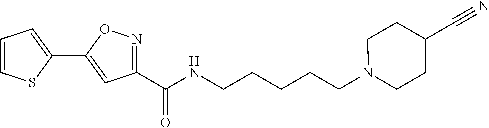 [SMILES: N#CC1CCN(CCCCCNC(=O)c2cc(-c3cccs3)on2)CC1] | MS (ESI) m/z 373.1 [M + H]⁺. ¹H NMR: (400 MHz, CDCl₃) δ ppm 7.54 (dd, J = 4.0, 1.2 Hz, 1H), 7.49 (dd, J = 4.8, 0.8 Hz, 1H), 7.20-7.30 (m, 1H), 7.16-7.12 (m, 1H), 6.86 (t, J = 4.8 Hz, 1H), 6.81 (s, 1H), 3.47-3.42 (m, 2H), 2.71-2.55 (m, 3H), 2.35-2.31 (m, 4H), 1.93-1.85 (m, 4H), 1.65-1.62 (m, 2H), 1.53-1.48 (m, 2H), 1.41-1.36 (m, 2H). |
| 57 | 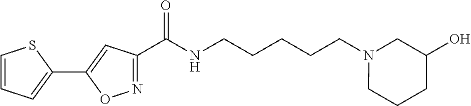 [SMILES: O=C(NCCCCCN1CCCC(O)C1)c1cc(-c2cccs2)on1] | MS (ESI) m/z 364.1 [M + H]⁺. ¹H NMR: (400 MHz, CDCl₃) δ ppm 7.54 (dd, J = 4.0, 1.2 Hz, 1H), 7.49 (dd, J = 4.8, 0.8 Hz, 1H), 7.15-7.13 (m, 1H), 6.86 (br, 1H), 6.81 (s, 1H), 3.89-3.79 (m, 1H), 3.48-3.43 ( m, 2H), 2.52-2.43 (m, 3H), 2.33 (t, J = 7.2 Hz, 2H), 2.28-2.24 (m, 1H), 1.81-1.77 (m, 1H), 1.68-1.60 (m, 3H), 1.56-1.50 (m, 4H), 1.42-1.37 (m, 2H). |
| 58 | 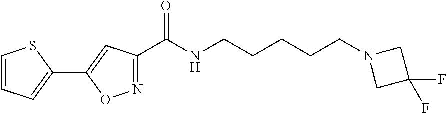 [SMILES: O=C(NCCCCCN1CC(F)(F)C1)c1cc(-c2cccs2)on1] | MS (ESI) m/z 355.9 [M + H]⁺. ¹H NMR (400 MHz, MeOD) δ ppm 7.71-7.67 (m, 2H), 7.23 (t, J = 5.2 Hz, 1H), 6.92 (s, 1H), 3.63 (t, J = 12.0 Hz, 4H), 3.41 (t, J = 7.2 Hz, 2H), 2.64-2.60 (m, 2H), 1.69-1.62 (m, 2H), 1.53-1.37 (m, 4H). |
| 59 | 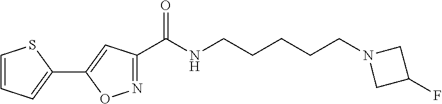 [SMILES: O=C(NCCCCCN1CC(F)C1)c1cc(-c2cccs2)on1] | MS (ESI) m/z 337.9 [M + H]⁺. ¹H NMR (400 MHz, CD₃OD) δ ppm 7.70-7.66 (m, 2H), 7.22 (t, J = 4.6 Hz, 1H), 6.92 (s, 1H), 5.22-5.03 (m, 1H), 3.72-3.54 (m, 2H), 3.40 (t, J = 6.8 Hz, 2H), 3.30-3.16 (m, 2H), 2.54 (t, J = 6.8 Hz, 2H), 1.68-1.61 (m, 2H), 1.51-1.34 (m, 4H). |
| 60 | 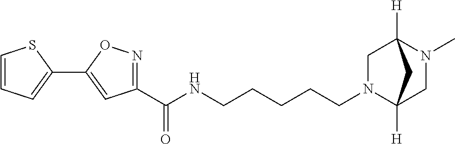 [SMILES: CN1C[C@H]2C[C@@H]1CN2CCCCCNC(=O)c1cc(-c2cccs2)on1] | MS (ESI) m/z 375.1 [M + H]⁺. ¹H NMR (400 MHz, DMSO-d₆) (t = 80° C.) δ ppm 8.50 (t, J = 4.0 Hz, 1H), 7.82 (d, J = 4.8 Hz, 1H), 7.72 (d, J = 2.8 Hz, 1H), 7.26-7.24 (m, 1H), 7.08 (s, 1H), 4.47-4.39 (m, 2H), 4.07-3.92 (m, 2H), 3.51-3.37 (m,2H), 3.36-3.21 (m, 6H), 2.88 (s, 3H), 1.85-1.71 (m, 2H), 1.64-1.57 (m, 2H), 1.44-1.37 (m, 2H). |
| 61 | 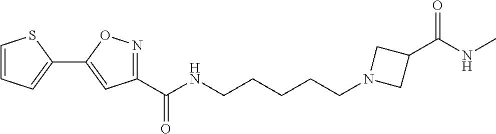 [SMILES: CNC(=O)C1CN(CCCCCNC(=O)c2cc(-c3cccs3)on2)C1] | MS (ESI) m/z 377.0 [M + Na]⁺. ¹H NMR (400 MHz, DMSO-d₆) δ ppm 8.80 (t, J = 5.6 Hz, 1H), 7.87 (dd, J = 4.8 Hz, 0.8 Hz, 1H), 7.79 (dd, J = 3.6 Hz, 0.8 Hz, 1H), 7.74-7.72 (m, 1H), 7.26 (dd, J = 4.8 Hz, 3.6 Hz, 1H), 7.17 (s, 1H), 3.29 (t, J = 6.8 Hz, 2H), 3.22 (q, J = 6.4 Hz, 2H), 3.08-3.03 (m, 1H), 3.01-2.96 (m, 2H), 2.55 (d, J = 4.8 Hz, 3H), 2.28 (t, J = 6.0 Hz, 2H), 1.51-1.46 (m, 2H), 1.26-1.24 (m, 4H). |
| 62 | 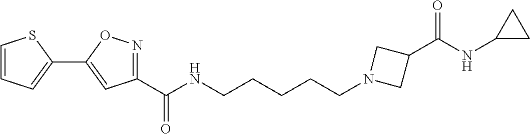 [SMILES: O=C(NCCCCCN1CC(C(=O)NC2CC2)C1)c1cc(-c2cccs2)on1] | MS (ESI) m/z 403.1 [M + H]⁺. ¹HNMR (400 MHz, DMSO-d₆) (t = 80° C.) δ ppm 8.51 (s, 1H), 8.08 (s, 1H), 7.83 (dd, J = 4.8 Hz, 0.8 Hz, 1H), 7.74 (dd, J = 3.6 Hz, 0.8 Hz, 1H), 7.27-7.24 (m, 1H), 7.07 (s, 1H), 4.17-4.01 (m, 4H), 3.49-3.40 (m, 1H), 3.31-3.26 (m, 2H), 3.13-3.11 (m, 2H), 2.71-2.65 (m, 1H), 1.61-1.53 (m, 4H), 1.40-1.32 (m, 2H), 0.67-0.62 (m, 2H), 0.47-0.42 (m, 2H). |
| 63 | 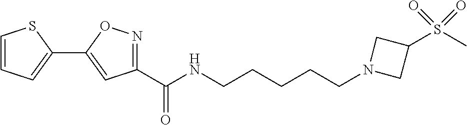 [SMILES: CS(=O)(=O)C1CN(CCCCCNC(=O)c2cc(-c3cccs3)on2)C1] | MS (ESI) m/z 398.0 [M + H]⁺. ¹H NMR: (400 MHz, DMSO-d₆) δ ppm 11.26-11.23 (m, 0.5H), 10.52-10.38 (m, 0.5H), 8.85 (d, J = 5.2 Hz, 1H), 7.88-7.78 (m, 2H), 7.35-7.09 (m, 3H), 4.52-4.43 (m, 5H), 3.27-3.06 (m, 7H), 1.52-1.47 (m, 4H), 1.33-1.30 (m, 2H). |
| 64 | 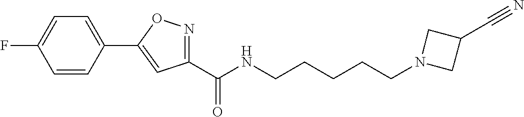 [SMILES: N#CC1CN(CCCCCNC(=O)c2cc(-c3ccc(F)cc3)on2)C1] | MS (ESI) m/z 357.1 [M + H]⁺. ¹H NMR (400 MHz,CD₃OD) δ ppm 7.95-7.89 (m, 2H), 7.27 (t, J = 8.8 Hz, 2H), 7.05 (s, 1H), 3.56 (t, J = 6.8 Hz, 2H), 3.44-3.32 (m ,5H), 2.51-2.44 (m, 2H), 1.68-1.58 (m, 2H), 1.45-1.34 (m, 4H). |
| 65 | 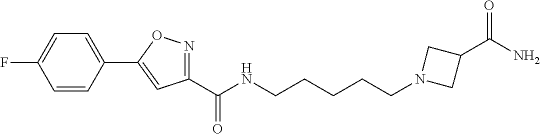 [SMILES: NC(=O)C1CN(CCCCCNC(=O)c2cc(-c3ccc(F)cc3)on2)C1] | MS (ESI) m/z 375.0 [M + H]⁺. ¹H NMR (400 MHz, CD₃OD) δ ppm 7.98-7.91 (m, 2H), 7.34-7.26 (m, 2H), 7.07 (s, 1H), 3.68-3.60 (m, 2H), 3.45-3.36 (m, 5H), 2.59 (t, J = 6.8 Hz, 2H), 1.72-1.62 (m, 2H), 1.51-1.37 (m, 4H). |
| 66 | 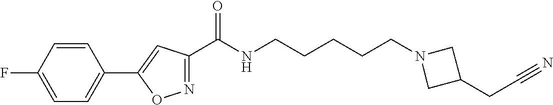 [SMILES: N#CCC1CN(CCCCCNC(=O)c2cc(-c3ccc(F)cc3)on2)C1] | MS (ESI) m/z 371.2 [M + H]⁺. ¹H NMR (400 MHz, CD₃OD) δ ppm 7.95-7.90 (m, 2H), 7.28 (t, J = 8 Hz, 2H), 7.05 (s, 1H), 3.49 (t, J = 8 Hz, 2H), 3.39 (t, J = 7.2 Hz, 2H), 2.98 (t, J = 7.6 Hz, 2H), 2.82-2.75 (m, 1H), 2.69 (d, J = 6.8 Hz, 2H), 2.49 (t, J = 6.8 Hz, 2H), 1.65-1.60 (m, 2H), 1.44-1.36 (m, 4H). |
| 67 | 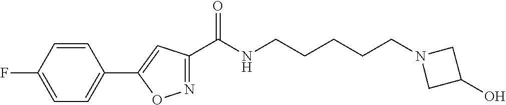 [SMILES: O=C(NCCCCCN1CC(O)C1)c1cc(-c2ccc(F)cc2)on1] | MS (ESI) m/z 348.2 [M + H]⁺. ¹H NMR (400 MHz, MeOD) δ ppm 7.96-7.89 (m, 2H), 7.27 (t, J = 8.8 Hz, 2H), 7.05 (s, 1H), 4.36-4.31 (m, 1H), 3.71-3.66 (m, 2H), 3.39 (t, J = 6.8 Hz, 2H), 2.95-2.90 (m, 2H), 2.54 (t, J = 6.4 Hz, 2H), 1.66-1.62 (m, 2H), 1.48-1.35 (m, 4H). |
| 68 | 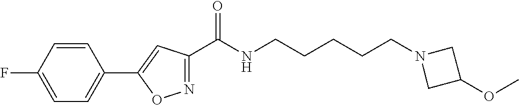 [SMILES: COC1CN(CCCCCNC(=O)c2cc(-c3ccc(F)cc3)on2)C1] | MS (ESI) m/z 362.2 [M + H]⁺. ¹H NMR (400 MHz, CD₃OD) δ ppm 7.94-7.90 (m, 2H), 7.27 (t, J = 8.8 Hz, 2H), 7.05 (s, 1H), 4.05-4.02 (m, 1H), 3.61-3.56 (m, 2H), 3.39 (t, J = 6.8 Hz, 2H), 3.24 (s, 3H), 2.98-2.93 (m, 2H), 2.50 (t, J = 6.8 Hz, 2H), 1.65-1.61 (m, 2H), 1.47-1.34 (m, 4H). |
| 69 | 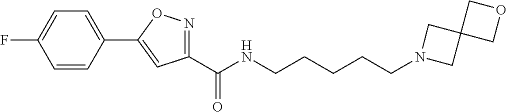 [SMILES: O=C(NCCCCCN1CC2(COC2)C1)c1cc(-c2ccc(F)cc2)on1] | MS (ESI) m/z 374.2 [M + H]⁺. ¹H NMR (400 MHz, CD₃OD) δ ppm 7.95-7.90 (m, 2H), 7.28 (t, J = 8.8 Hz, 2H), 7.05 (s, 1H), 4.72 (s, 4H), 3.41-3.36 (m, 6H), 2.43 (t, J = 6 Hz, 2H), 1.67-1.58 (m, 2H), 1.44-1.34 (m, 4H). |
| 70 | 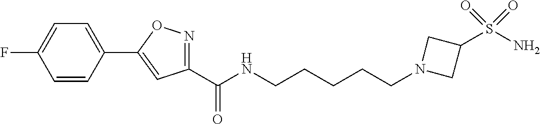 [SMILES: NS(=O)(=O)C1CN(CCCCCNC(=O)c2cc(-c3ccc(F)cc3)on2)C1] | MS (ESI) m/z 411.2 [M + H]⁺. ¹H NMR (400 MHz, CD₃OD) δ ppm 7.96-7.93 (m, 2H), 7.32-7.28 (m, 2H), 7.07 (s, 1H), 4.07-4.05 (m, 1H), 3.67-3.65 (m, 2H), 3.49-3.47 (m, 2H), 3.41-3.39 (m, 2H), 2.58-2.55 (m, 2H), 1.68-1.64 (m, 2H), 1.44-1.40 (m, 4H). |
| 71 | 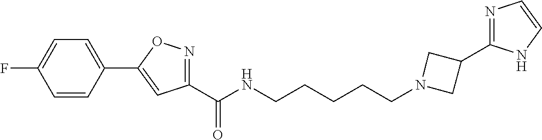 [SMILES: O=C(NCCCCCN1CC(c2ncc[nH]2)C1)c1cc(-c2ccc(F)cc2)on1] | MS (ESI) m/z 398.2 [M + H]⁺. ¹H NMR (400 MHz, CD₃OD) δ ppm 7.94-7.91 (m, 2H), 7.30-7.26 (m, 2H), 7.05 (s, 1H), 6.96 (s, 2H), 3.77-3.74 (m, 3H), 3.40-3.38 (m, 2H), 3.37-3.32 (m, 2H), 2.60-2.57 (m, 2H), 1.67-1.63 (m, 2H), 1.45-1.42 (m, 4H). |
| 72 | 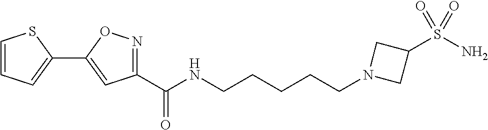 [SMILES: NS(=O)(=O)C1CN(CCCCCNC(=O)c2cc(-c3cccs3)on2)C1] | MS (ESI) m/z 399.2 [M + H]⁺. ¹H NMR (400 MHz, DMSO-d₆) δ ppm 7.69-7.66 (m, 2H), 7.21 (dd, J = 3.6 Hz, 8.8 Hz, 1H), 6.91 (s, 1H), 4.05-4.03 (m, 1H), 3.65-3.63 (m, 2H), 3.47-3.45 (m, 2H), 3.39-3.38 (m, 2H), 2.56-2.53 (m, 2H), 1.65-1.60 (m, 2H), 1.42-1.38 (m, 4H). |

### Example 73: 5-(5-Fluorothiophen-2-yl)-N-(5-(4-methylpiperazin-1-yl)pentyl)isoxazole-3-carboxamide

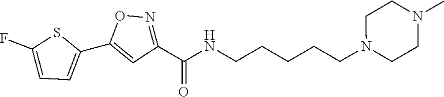 [SMILES: CN1CCN(CCCCCNC(=O)c2cc(-c3ccc(F)s3)on2)CC1]

### Step 1: Preparation of 5-fluoro-N-methoxy-N-methylthiophene-2-carboxamide

To a solution of 5-fluorothiophene-2-carboxylic acid (3.0 g, 20.4 mmol, 1.0 eq.) in THF (300 mL) was added N,O-dimethylhydroxylamine hydrochloride (3.99 g, 40.8 mmol, 2.0 eq), HOBt (4.11 g, 30.6 mmol, 1.5 eq), DIEA (10.5 g, 81.6 mmol, 4.0 eq) and EDCI (7.83 g, 40.8 mmol, 2.0 eq) under N₂ protection at 0° C. The mixture was allowed to warm to 20° C. for about 8 hours. Then the mixture was quenched with H₂O (100 mL), extracted with EtOAc. The combined organic phase was dried over anhydrous sodium sulfate, filtered and concentrated in vacuo to give the crude product, which was purified by silica gel chromatography eluting with 15% EtOAc in hexane to give the title compound (3.5 g, 90% yield) as a yellow oil. MS (ESI) m/z 189.8 [M+H]⁺. ¹H NMR (400 MHz, DMSO-d₆) δ ppm δ 7.60 (t, J=4.4 Hz, 1H), 7.95 (d, J=7.2 Hz, 1H), 3.77 (s, 3H), 3.26 (s, 3H).

### Step 2: Preparation of 1-(5-fluorothiophen-2-yl)ethan-1-one

To a stirred solution of 5-fluoro-N-methoxy-N-methylthiophene-2-carboxamide (3.5 g, 18.5 mmol, 1.0 eq.) in THF (30 mL) was added compound MeMgCl (3 M solution in THF, 9.25 mL, 27.75 mmol, 1.5 eq) over a period of 25 minutes at 0° C. under N₂ protection, while maintaining the internal temperature below 10° C. The cooling bath was removed and the solution was allowed to warm to room temperature over 1 hour. Then the reaction mixture was quenched by a saturated solution of ammonium chloride (30 mL) and was stirred for 10 minutes. The mixture was extracted with EtOAc and the combined extracts were dried over anhydrous sodium sulfate, filtered and concentrated to give the crude product as a yellow oil which was purified by silica gel chromatography eluting with 15% EtOAc in hexane to give the title compound (2.0 g, 75% yield) as a yellow oil. MS (ESI) m/z 144.8 [M+H]⁺. ¹H NMR (400 MHz, DMSO-d₆) δ 7.67 (t, J=4.0 Hz, 1H), 7.95 (dd, J=1.2 Hz, 4.4 Hz, 1H), 2.48 (s, 3H).

### Step 3: Preparation of ethyl 4-(5-fluorothiophen-2-yl)-2,4-dioxobutanoate

To a solution of 1-(5-fluorothiophen-2-yl)ethan-1-one (1.5 g, 10 mmol, 1.0 eq.) and (CO₂Et)₂ (1.75 g, 12 mmol, 1.2 eq.) in toluene (30 mL) was added t-BuOK (1.35 g, 12 mmol, 1.2 eq.). The reaction mixture was stirred at 25° C. for 4 hours. The mixture was quenched with 1N HCl to pH 4. The solution was transferred to a separatory funnel. The organic layer was washed with H₂O, followed by brine, dried over anhydrous sodium sulfate, filtered and concentrated in vacuo to give the crude compound which was purified by HPLC to give the compound (1.5 g, 60% yield) as a yellow solid. MS (ESI) m/z 244.8 [M+H]⁺. ¹H NMR (400 MHz, DMSO-d₆) δ 8.15 (s, 1H), 7.05 (br s, 1H), 7.02 (d, J=3.2 Hz, 1H), 4.30 (q, J=6.8 Hz, 2H), 1.30 (t, J=6.8 Hz, 3H).

### Step 4: Preparation of ethyl 5-(5-fluorothiophen-2-yl)isoxazole-3-carboxylate

To a solution of ethyl 4-(5-fluorothiophen-2-yl)-2,4-dioxobutanoate (500 mg, 5.10 mmol, 1.0 eq.) in EtOH (60 mL) was added NH₂OH.HCl (285 mg, 8.2 mmol, 2.0 eq.). The reaction mixture was stirred at 90° C. for 16 hours. The reaction mixture was concentrated and the residue was dissolved in EtOAc (30 mL). The mixture was washed with H₂O (30 mL) and brine (30 mL), dried over anhydrous sodium sulfate, filtered and concentrated to obtain the crude product, which was purified by silica gel chromatography eluting with 6% EtOAc in hexane to give the title compound (400 mg, 81% yield) as yellow oil. MS (ESI) m/z 241.8 [M+H]⁺.

]⁺. ¹H NMR (400 MHz, DMSO-d₆) δ 7.60 (t, J=8.0 Hz, 1H), 7.33 (s, 1H), 6.98 (dd, J=2.0 Hz, 4.0 Hz, 1H), 4.38 (q, J=6.8 Hz, 2H), 1.33 (t, J=7.2 Hz, 3H).

### Step 5: Preparation of 5-(5-fluorothiophen-2-yl)-N-(5-(4-methylpiperazin-1-yl)pentyl)isoxazole-3-carboxamide

To a solution of ethyl 5-(5-fluorothiophen-2-yl)isoxazole-3-carboxylate (500 mg, 2.07 mmol, 1.0 eq.) and 5-(4-methylpiperazin-1-yl)pentan-1-amine (382.6 mg, 2.07 mmol, 1.0 eq.) in THF (30 mL) was added TEA (626.3 mg, 6.21 mmol, 3.0 eq.). The mixture was cooled to 0° C. Me₃Al (2M in toluene, 10 mL, 20.7 mmol, 10.0 eq.) was added dropwise, then the mixture was stirred at 22-29° C. for 16 hours. The mixture was quenched with H₂O (30 mL) and filtered though a Celite Pad. The filtration was concentrated to obtained the crude product, which was purified by pre-HPLC to give the title compound (261 mg, 33% yield) as a white solid. MS (ESI) m/z 241.8 [M+H]⁺. ¹H NMR (400 MHz, CDCl₃) δ 7.17 (t, J=4.0 Hz, 1H), 6.81 (br s, 1H), 6.73 (s, 1H), 6.56 (dd, J=1.2 Hz, 4.0 Hz, 1H), 3.44 (q, J=6.4 Hz, 2H), 2.48-2.33 (m, 10H), 2.29 (s, 3H), 1.67-1.60 (m, 2H), 1.58-1.51 (m, 2H), 1.43-1.36 (m, 2H).

### Example 74: N-(3,3-Difluoro-5-(4-methylpiperazin-1-yl)pentyl)-5-(thiophen-2-yl)isoxazole-3-carboxamide

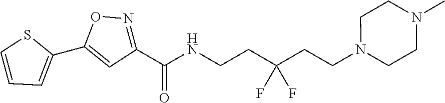 [SMILES: CN1CCN(CCC(F)(F)CCNC(=O)c2cc(-c3cccs3)on2)CC1]

### Step 1: Preparation of tert-Butyl 3-(5-(thiophen-2-yl)isoxazole-3-carboxamido)propanoate

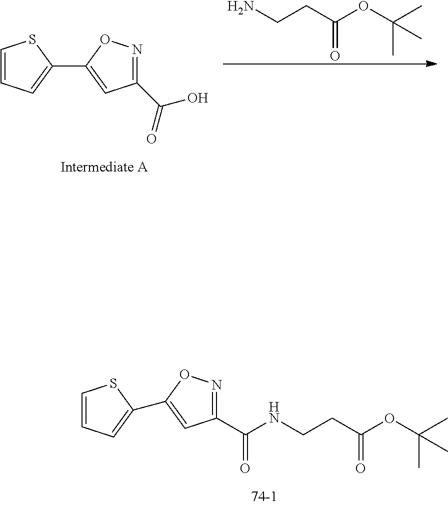

To a solution of Intermediate A (1.0 g, 5.12 mmol, 1.0 eq) in DCM (10 mL anhydrous) were added (COCl)₂ (779.2 mg, 6.14 mmol, 1.2 eq) and DMF (0.1 mL, anhydrous, catalytic amount). Then the mixture was stirred at 18° C. for 1 hour and the mixture was concentrated to give the yellow solid. Then the solid was dissolved in DCM (5.0 mL, anhydrous) and the mixture was added to a solution of tert-butyl 3-aminopropanoate (743.4 mg, 5.12 mmol, 1.0 eq) and triethylamine (1.04 g, 10.24 mmol, 2.0 eq) in DCM (5.0 mL, anhydrous) dropwise over 3 minutes. After that, the mixture was stirred at 18° C. for 16 hours. The mixture was concentrated to give the crude product which was purified by silica gel chromatography eluting with 20% EtOAc in petroleum ether to afford the title compound (1.5 g, 90.9% yield) as a yellow solid. MS (ESI) m/z 344.9 [M+Na]⁺.

¹H NMR (400 MHz, CDCl₃) δ ppm 7.53 (d, J=3.6 Hz, 1H), 7.48 (d, J=4.8 Hz, 1H), 7.35 (br, 1H), 7.13 (t, J=4.0 Hz, 1H), 6.80 (s, 1H), 3.71-3.66 (m, 2H), 2.56 (t, J=6.0 Hz, 2H), 1.46 (s, 9H).

### Step 2: Preparation of 3-(5-(Thiophen-2-yl)isoxazole-3-carboxamido)propanoic Acid

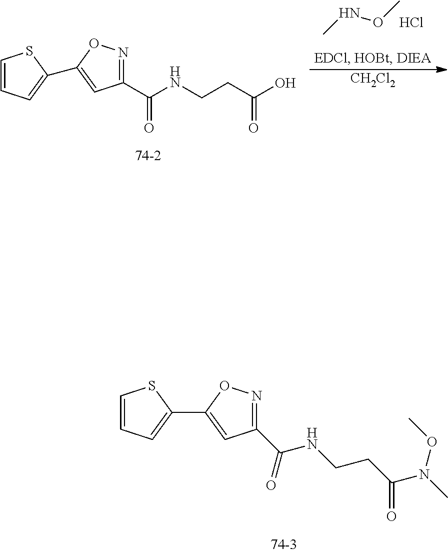

To a solution of compound 74-1 (500 mg, 1.55 mmol, 1.0 eq) in DCM (6.0 mL, anhydrous) was added TFA (2.0 mL) and the mixture was stirred at 15° C. for 1.5 hours. The mixture was concentrated to give the title compound (412.8 mg, 100% yield) as a yellow solid, which was used to next step without further purification. MS (ESI) m/z 267.0 [M+H]⁺.

### Step 3: Preparation of N-(3-(Methoxy(methyl)amino)-3-oxopropyl)-5-(thiophen-2-yl)isoxazole-3-carboxamide

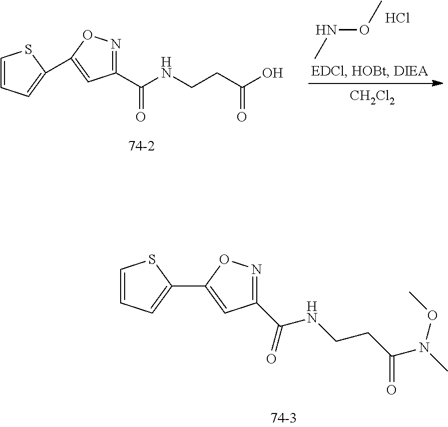

To a solution of compound 74-2 (825.5 mg, 3.1 mmol, 1.0 eq), N, O-dimethylhydroxylamine hydrochloride (362.7 mg, 3.72 mmol, 1.2 eq) and DIEA (2.0 g, 15.5 mmol, 5.0 eq) in DCM (30 mL anhydrous) were added EDCI (892.8 mg, 4.65 mmol, 1.5 eq) and HOBt (628.2 mg, 4.65 mmol, 1.5 eq). Then the mixture was stirred at 15° C. for 16 hours. The mixture was quenched with water (20 mL) and the organic phase was separated, washed with brine (20 mL), dried over anhydrous Na₂SO₄, filtered and concentrated to give the crude product which was purified by silica gel chromatography eluting with 1% methanol in DCM to give the title compound (1.1 g, 76.4% yield) as a yellow solid. MS (ESI) m/z 309.9 [M+H]⁺. ¹H NMR (400 MHz, CDCl₃) δ ppm 7.53-7.47 (m, 3H), 7.13 (t, J=4.0 Hz, 1H), 6.79 (s, 1H), 3.78-3.73 (m, 2H), 3.67 (s, 3H), 3.19 (s, 3H), 2.77 (brs, 2H).

### Step 4: Preparation of N-(3-Oxopent-4-en-1-yl)-5-(thiophen-2-yl)isoxazole-3-carboxamide

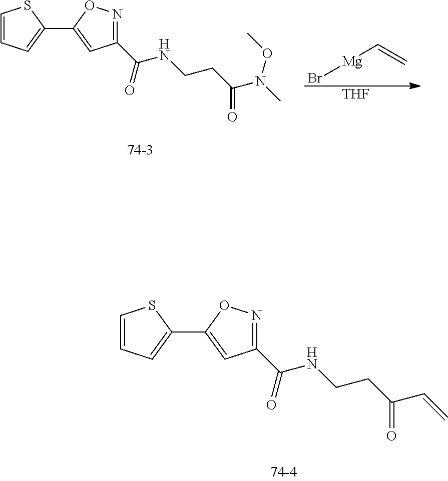

To a solution of compound 74-3 (1.1 g, 3.56 mmol, 1.0 eq) in THF (10 mL anhydrous) was added vinyl magnesium bromide (14.2 mL, 14.2 mmol, 4.0 eq, 1.0 M in tetrahydrofuran) dropwise at 0° C. over 5 minutes. Then the mixture was stirred at 0° C. for 2 hours. The mixture was quenched with NH₄Cl (20 mL aqueous) at 0° C. and extracted with EtOAc (2*30 mL). The combined organic phase was washed with brine (30 mL), dried over anhydrous Na₂SO₄, filtered and concentrated to give the crude product which was purified by silica gel chromatography eluting with petroleum ether/EtOAc from 6/1 to 3/1 to give the title compound (450 mg, 45.7% yield) as a yellow solid. MS (ESI) m/z 276.9 [M+H]⁺. ¹H NMR (400 MHz, CDCl₃) δ ppm 7.53 (dd, J=3.6 Hz, 1.2 Hz, 1H), 7.48 (dd, J=5.2 Hz, 1.2 Hz, 1H), 7.32 (brs, 1H), 7.15-7.12 (m, 1H), 6.79 (s, 1H), 6.39-6.34 (m, 1H), 6.26 (dd, J=18.0 Hz, 1.2 Hz, 1H), 5.91 (dd, J=10.4 Hz, 1.2 Hz, 1H), 3.79-3.74 (m, 2H), 2.97 (t, J=5.6 Hz, 2H).

### Step 5: Preparation of tert-Butyl 4-(3-oxo-5-(5-(thiophen-2-yl)isoxazole-3-carboxamido)pentyl)piperazine-1-carboxylate

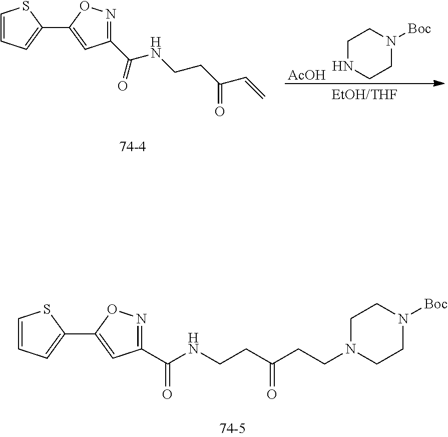

To a solution of compound 74-4 (2.69 mg, 14.45 mmol, 5.0 eq), AcOH (0.5 mL, catalytic amount) in THF (10.0 mL anhydrous) and ethanol (10.0 mL anhydrous) was added a solution of tert-butyl piperazine-1-carboxylate (800 mg, 2.89 mmol, 1.0 eq) in THF (10.0 mL anhydrous) dropwise over 3 minutes. After that, the mixture was stirred at 30° C. for 3 hours. The mixture was concentrated and the residue was dissolved with EtOAc (40 mL), washed with sodium bicarbonate (20 mL, saturated), brine (40 mL), dried over anhydrous Na₂SO₄, filtered and concentrated to give the crude product which was purified by silica gel chromatography eluting with DCM/MeOH from 200/1 to 50/1 to give the title compound (1.2 g, 90.2% yield) as a yellow solid. MS (ESI) m/z 463.1 [M+H]⁺.

¹H NMR (400 MHz, CDCl₃) δ ppm 7.53 (dd, J=4.0 Hz, 1.2 Hz, 1H), 7.48 (dd, J=5.2 Hz, 1.6 Hz, 1H), 7.27 (brs, 1H), 7.15-7.12 (m, 1H), 6.78 (s, 1H), 3.72-3.67 (m, 2H), 3.38 (t, J=4.8 Hz, 4H), 2.81 (t, J=6.0 Hz, 2H), 2.70-2.68 (m, 2H), 2.63-2.59 (m, 2H), 2.38-2.36 (m, 4H), 1.43 (s, 9H).

### Step 6: Preparation of tert-Butyl 4-(3,3-difluoro-5-(5-(thiophen-2-yl)isoxazole-3-carboxamido)pentyl)piperazine-1-carboxylate

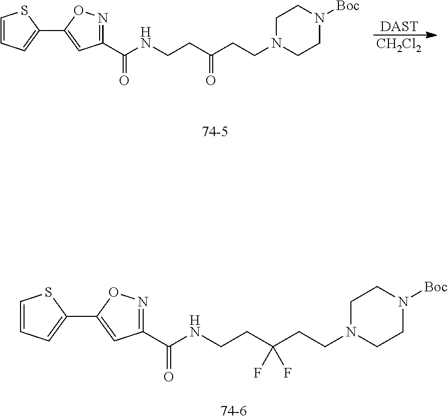

To a solution of compound 74-5 (130 mg, 0.28 mmol, 1.0 eq) in DCM (10.0 mL, anhydrous) was DAST (902.7 mg, 5.6 mmol, 20.0 eq) at −78° C. and the mixture was stirred from −78° C. to 24° C. for 16 hours. The mixture was poured into ice-cold NaHCO₃ (saturated aqueous, 200 mL) and filtered. After that, the organic phase was separated and the aqueous phase was extracted with DCM (2*50 mL). The combined organic phase was washed with brine (100 mL), dried over anhydrous Na₂SO₄, filtered and concentrated to give the crude product which was purified by preparative HPLC (column: Kromasil 150*25 mm*10 um, gradient: 50-60% B (A=0.05% ammonia hydroxide/water, B=acetonitrile)) to give the title compound (18 mg, 13.2% yield) as a yellow solid. MS (ESI) m/z 485.1 [M+H]⁺. ¹H NMR (400 MHz, CDCl₃) δ ppm 7.55 (dd, J=3.6 Hz, 1.2 Hz, 1H), 7.50 (dd, J=5.2 Hz, 1.2 Hz, 1H), 7.16-7.14 (m, 1H), 7.05 (t, J=6.0 Hz, 1H), 6.81 (s, 1H), 3.72-3.67 (m, 2H), 3.44-3.42 (m, 4H), 2.58 (t, J=7.6 Hz, 2H), 2.42-2.40 (m, 4H), 2.27-2.05 (m, 4H), 1.45 (s, 9H). ¹⁹F NMR (400 MHz, CDCl₃) δ ppm −97.54.

### Step 7: Preparation of N-(3,3-Difluoro-5-(piperazin-1-yl)pentyl)-5-(thiophen-2-yl)isoxazole-3-carboxamide

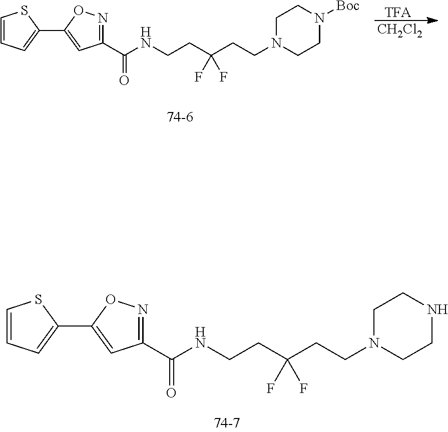

To a solution of compound 74-6 (100 mg, 0.20 mmol, 1.0 eq) in DCM (3.0 mL, anhydrous) was added TFA (1.5 mL) and the mixture was stirred at 32° C. for 30 minutes. The mixture was concentrated to give the crude title compound (76.8 mg, 100% yield) as a yellow oil, which was used to next step without further purification. MS (ESI) m/z 385.1 [M+H]⁺.

### Step 8: N-(3,3-Difluoro-5-(4-methylpiperazin-1-yl)pentyl)-5-(thiophen-2-yl)isoxazole-3-carboxamide

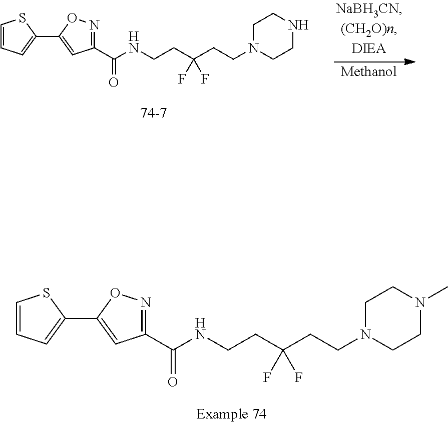 [SMILES: O=C(NCCC(F)(F)CCN1CCNCC1)c1cc(-c2cccs2)on1 ; CN1CCN(CCC(F)(F)CCNC(=O)c2cc(-c3cccs3)on2)CC1]

To a solution of compound 74-7 (76.8 mg, 0.20 mmol, 1.0 eq), paraformaldehyde (30 mg, 1.0 mmol, 5.0 eq) and DIEA (77.5 mg, 0.6 mmol, 3.0 eq) in MeOH (5.0 mL, anhydrous) was added sodium cyanoborohydride (62.8 mg, 1.0 mmol, 5.0 eq) and the mixture was stirred at 32° C. for 1 hour. The mixture was quenched with water (5.0 mL) and extracted with DCM (2*20 mL). The combined organic phase was concentrated to give the crude which was purified by preparative HPLC (column: Xtimate C18 150*25 mm*5 um, gradient: 25-55% B (A=0.05% ammonia hydroxide/water, B=acetonitrile)) to give the title compound (34.2 mg, 42.9% yield) as a light yellow solid. MS (ESI) m/z 399.2 [M+H]⁺. ¹H NMR (400 MHz, CDCl₃) δ ppm 7.54 (dd, J=4.0 Hz, 1.2 Hz, 1H), 7.49 (dd, J=5.2 Hz, 1.2 Hz, 1H), 7.16-7.14 (m, 1H), 7.06 (t, J=6.0 Hz, 1H), 6.81 (s, 1H), 3.72-3.67 (m, 2H), 2.58-2.40 (m, 10H), 3.29 (s, 3H), 2.27-2.04 (m, 4H). ¹⁹F NMR (400 MHz, DMSO-d₆) δ ppm −94.41.

### Example 75: N-(5-(3-Carbamoylazetidin-1-yl)-3,3-difluoropentyl)-5-(thiophen-2-yl)isoxazole-3-carboxamide

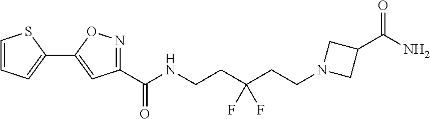 [SMILES: NC(=O)C1CN(CCC(F)(F)CCNC(=O)c2cc(-c3cccs3)on2)C1]

### Step 1: Preparation of Methyl 1-(3-oxo-5-(5-(thiophen-2-yl)isoxazole-3-carboxamido)pentyl)azetidine-3-carboxylate

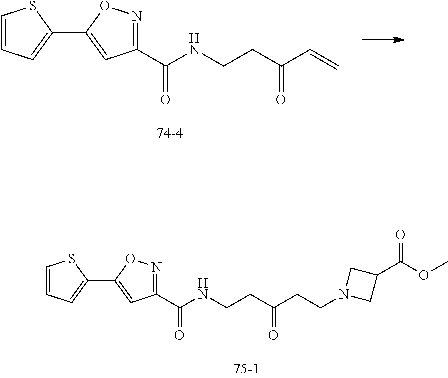 [SMILES: C=CC(=O)CCNC(=O)c1cc(-c2cccs2)on1 ; COC(=O)C1CN(CCC(=O)CCNC(=O)c2cc(-c3cccs3)on2)C1]

The title compound was prepared by using a procedure similar to that of compound 74-5 by replacing of tert-butyl piperazine-1-carboxylate with methyl azetidine-3-carboxylate hydrochloride as a yellow oil. MS (ESI) m/z 392.0 [M+H]⁺. ¹H NMR (400 MHz, CDCl₃) δ ppm 7.53 (dd, J=3.6 Hz, 0.8 Hz, 1H), 7.48 (dd, J=5.2 Hz, 1.6 Hz, 1H), 7.42 (brs, 1H), 7.15-7.13 (m, 1H), 6.79 (s, 1H), 3.72-3.68 (m, 2H), 3.69 (s, 3H), 3.54-3.50 (m, 2H), 3.33-3.24 (m, 3H), 2.79-2.72 (m, 4H), 2.46 (t, J=6.8 Hz, 2H).

### Step 2: Preparation of Methyl 1-(3,3-difluoro-5-(5-(thiophen-2-yl)isoxazole-3-carboxamido)pentyl)azetidine-3-carboxylate

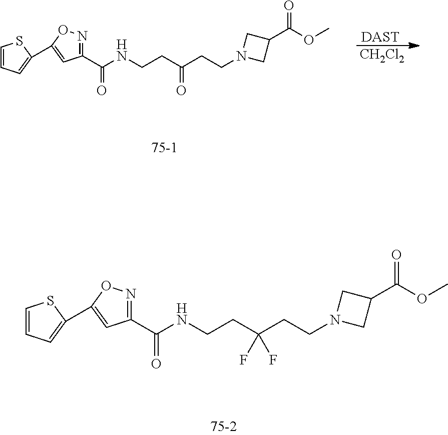

The title compound was prepared by using a procedure similar to that of compound 74-6 by replacing of compound 74-5 with compound 75-1 as a yellow solid. MS (ESI) m/z 414.0 [M+H]⁺.

### Step 3: Preparation of 1-(3,3-Difluoro-5-(5-(thiophen-2-yl)isoxazole-3-carboxamido)pentyl)azetidine-3-carboxylic Acid

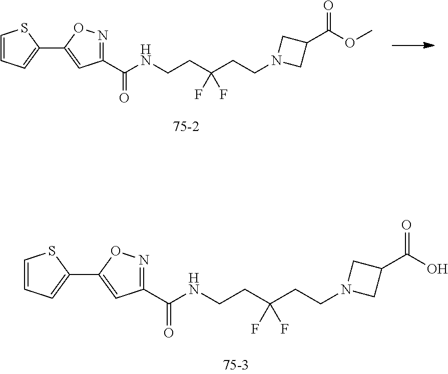 [SMILES: COC(=O)C1CN(CCC(F)(F)CCNC(=O)c2cc(-c3cccs3)on2)C1 ; O=C(NCCC(F)(F)CCN1CC(C(=O)O)C1)c1cc(-c2cccs2)on1]

A mixture of compound 75-2 (50 mg, 0.12 mmol, 1.0 eq) in ammonia hydroxide (3.0 mL, 25%-28% wt) was stirred under microwave irradiation at 60° C. for 1 hour. The mixture was concentrated to give the crude title compound (40 mg, 83.6% yield) as a yellow solid, which was used to next step without further purification. MS (ESI) m/z 400.1 [M+H]⁺.

### Step 4: Preparation of N-(5-(3-Carbamoylazetidin-1-yl)-3,3-difluoropentyl)-5-(thiophen-2-yl)isoxazole-3-carboxamide

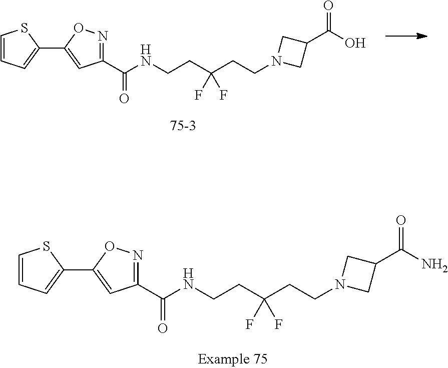 [SMILES: O=C(NCCC(F)(F)CCN1CC(C(=O)O)C1)c1cc(-c2cccs2)on1 ; NC(=O)C1CN(CCC(F)(F)CCNC(=O)c2cc(-c3cccs3)on2)C1]

To a solution of compound 75-3 (40 mg, 0.10 mmol, 1.0 eq), NH₄Cl (16.0 mg, 0.30 mmol, 3.0 eq) and DIEA (38.7 mg, 0.30 mmol, 3.0 eq) in DMF (3.0 mL, anhydrous) was added HATU (57.3 mg, 0.15 mmol, 1.5 eq) and the mixture was stirred at 34° C. for 16 hours. The mixture was concentrated and the residue was purified by preparative HPLC (column: Xtimate C18 150*25 mm*5 um, gradient: 23-53% B (A=0.05% ammonia hydroxide/water, B=acetonitrile)) to give the title compound (8.8 mg, 22.1% yield) as a white solid. MS (ESI) m/z 399.1 [M+H]⁺. ¹H NMR (400 MHz, DMSO-d₆) δ ppm 8.90 (t, J=4.8 Hz, 1H), 7.87 (d, J=4.4 Hz, 1H), 7.80 (d, J=3.2 Hz, 1H), 7.28-7.26 (m, 2H), 7.19 (s, 1H), 6.84 (br, 1H), 3.45-3.41 (m, 2H), 3.28 (br, 2H), 3.05-3.01 (m, 3H), 2.46-2.44 (m, 2H), 2.25-2.05 (m, 2H), 1.98-1.82 (m, 2H). ¹⁹F NMR (400 MHz, DMSO-d₆) δ ppm 94.48.

### Example 76: N-(5-(3-Carbamoylazetidin-1-yl)-3,3-difluoropentyl)-5-(4-fluorophenyl)isoxazole-3-carboxamide

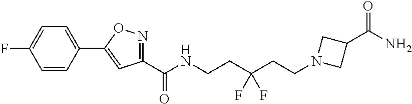 [SMILES: NC(=O)C1CN(CCC(F)(F)CCNC(=O)c2cc(-c3ccc(F)cc3)on2)C1]

The title compound was prepared by using a procedure similar to that of compound 74-4 and Example 75 by replacing of 5-(thiophen-2-yl)isoxazole-3-carboxylic acid with 5-(4-fluorophenyl)isoxazole-3-carboxylic acid as a white solid. MS (ESI) m/z 411.2 [M+H]⁺. ¹H NMR (400 MHz, CDCl₃) δ ppm 7.81-7.77 (m, 2H), 7.21-7.13 (m, 3H), 6.90 (s, 1H), 6.19 (br, 1H), 5.30 (br, 1H), 3.72-3.67 (m, 2H), 3.46-3.42 (m, 2H), 3.40-3.36 (m, 2H), 3.13-3.06 (m, 1H), 2.66 (t, J=7.6 Hz, 2H), 2.28-2.16 (m, 2H), 2.00-1.89 (m, 2H). ¹⁹F NMR (400 MHz, CDCl₃) δ ppm −108.36, 97.18.

### Example 77: N-(5-(3-((Cyanomethyl)carbamoyl)azetidin-1-yl)pentyl)-5-(thiophen-2-yl)isoxazole-3-carboxamide

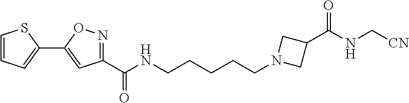 [SMILES: N#CCNC(=O)C1CN(CCCCCNC(=O)c2cc(-c3cccs3)on2)C1]

### Step 1: Preparation of Methyl 1-(5-(5-(thiophen-2-yl)isoxazole-3-carboxamido)pentyl)azetidine-3-carboxylate

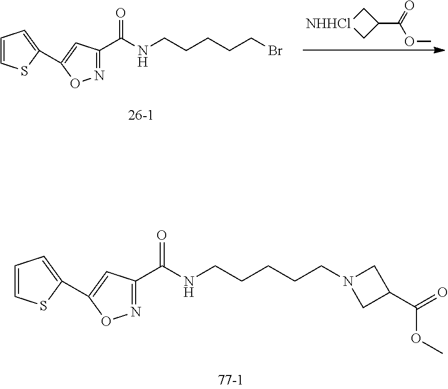

To a suspension of compound 26-1 (2 g, 5.83 mmol, 1 eq) in CH₃CN (20 mL) was added K₂CO₃ (2.42 g, 17.48 mmol, 3 eq) and KI (968 mg, 5.83 mmol, 1 eq) at 0° C. After addition, methyl azetidine-3-carboxylate hydrochloride (1.80 g, 11.65 mmol, 2.0 eq) was added and the mixture was stirred at 30° C. for 18 hours. The mixture was filtered. The filtrate was concentrated under reduced pressure to give the crude title compound (2.41 g) as a light yellow oil. MS (ESI) m/z 378.0 [M+H]⁺.

### Step 2: Preparation of 1-(5-(5-(Thiophen-2-yl)isoxazole-3-carboxamido)pentyl)azetidine-3-carboxylic Acid

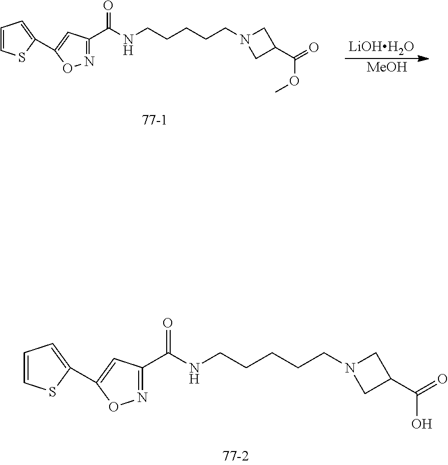

To a stirred solution of compound 77-1 (2.38 g, 6.31 mmol, 1.0 eq) in H₂O/MeOH (8 mL/16 mL) was added LiOH*H₂O (529 mg, 12.61 mmol, 2.0 eq) at 0° C. Then the mixture was stirred at 28° C. for 1.5 hours. Acidify the reaction mixture by adding, with stirring, 14 mL of 1N HCl to pH 5-6, and then extracted with EtOAc (3*25 mL). The combined organic layers were dried over anhydrous Na₂SO₄, filtered and concentrated under reduced pressure to afford the crude title compound (1.8 g, 78.55% yield) as a yellow gum which was used without further purification. MS (ESI) m/z 364.1 [M+H]⁺. ¹H NMR (400 MHz, CD₃OD) δ ppm 7.71-7.68 (m, 2H), 7.23 (dd, J=3.6 Hz, 4.8 Hz, 1H), 6.93 (s, 1H), 4.23-4.21 (m, 4H), 3.45-3.34 (m, 3H), 3.20-3.18 (m, 2H), 1.71-1.61 (m, 4H), 1.47-1.44 (m, 2H).

### Step 3: Preparation of N-(5-(3-((Cyanomethyl)carbamoyl)azetidin-1-yl)pentyl)-5-(thiophen-2-yl)isoxazole-3-carboxamide

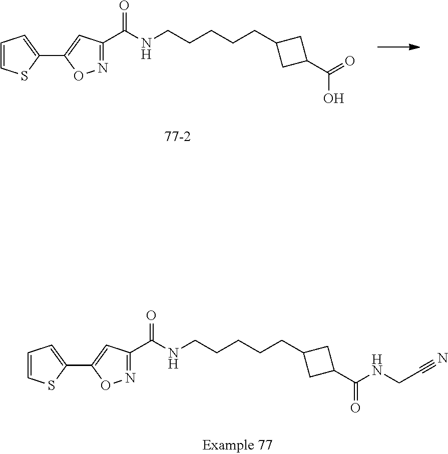 [SMILES: O=C(NCCCCCC1CC(C(=O)O)C1)c1cc(-c2cccs2)on1 ; N#CCNC(=O)C1CC(CCCCCNC(=O)c2cc(-c3cccs3)on2)C1]

To a solution of 77-2 (70 mg, 0.192 mmol, 1.0 eq) in DMF (1 mL) was added compound 2-aminoacetonitrile (53.5 mg, 0.577 mmol, 3.0 eq), DIEA (124.5 mg, 0.963 mmol, 5.0 eq), HATU (146.4 mg, 0.385 mmol, 2.0 eq). The mixture was stirred at 27° C. for 14 hours. The mixture was filtered and the filtrate was purified by preparative HPLC (Xtimate C18 150*25 mm*5 um, gradient: 20-50% B (A=0.05% HCl/water, B=CH₃CN), flow rate: 25 mL/min) to afford the title compound (23.2 mg, 30% yield) as an off-white solid. MS (ESI) m/z 402.1 [M+H]⁺. ¹H NMR (400 MHz, CD₃OD) δ ppm 7.71-7.68 (m, 2H), 7.24-7.22 (m, 1H), 6.92 (s, 1H), 4.17 (s, 2H), 3.55-3.54 (m, 2H), 3.42-3.40 (m, 2H), 3.33-3.29 (m, 3H), 2.53-2.49 (m, 2H), 1.67-1.63 (m, 2H), 1.433-1.40 (m, 4H).

### Example 78: N-(5-(3-((2-Hydroxyethyl)carbamoyl)azetidin-1-yl)pentyl)-5-(thiophen-2-yl)isoxazole-3-carboxamide

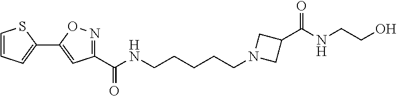 [SMILES: O=C(NCCCCCN1CC(C(=O)NCCO)C1)c1cc(-c2cccs2)on1]

The title compound was prepared by using a procedure similar to that of Example 77 by replacing of 2-aminoacetonitrile with 2-aminoethan-1-ol as a light yellow solid. MS (ESI) m/z 407.1 [M+H]⁺. ¹H NMR (400 MHz, CDCl₃) δ ppm 7.55 (dd, J=1.6 Hz, 4 Hz, 1H), 7.50 (d, J=5.2 Hz, 1H), 7.16 (d, J=5.2 Hz, 1H), 6.97 (m, 2H), 6.82 (s, 1H), 3.78-3.75 (m, 2H), 3.47-3.45 (m, 4H), 3.35-3.33 (m, 4H), 3.07-3.05 (m, 1H), 2.46-2.42 (m, 2H), 1.43-1.41 (m, 2H) 1.40-1.39 (m, 2H).

### Example 79: N-(5-(3-(((1,3-cis)-3-Hydroxycyclobutyl)carbamoyl)azetidin-1-yl)pentyl)-5-(thiophen-2-yl)isoxazole-3-carboxamide

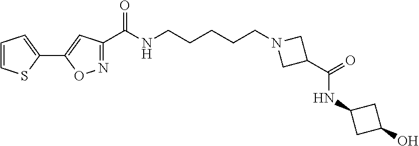 [SMILES: O=C(NCCCCCN1CC(C(=O)N[C@H]2C[C@@H](O)C2)C1)c1cc(-c2cccs2)on1]

The title compound was prepared by using a procedure similar to that of Example 77 by replacing of 2-aminoacetonitrile with (1,3-cis)-3-aminocyclobutan-1-ol as a white solid. MS (ESI) m/z 433.1 [M+H]⁺. ¹H NMR (400 MHz, CDCl₃) δ ppm 7.55 (dd, J=1.2 Hz, 4 Hz, 1H), 7.51 (dd, J=0.8 Hz, 4.8 Hz, 1H), 7.15 (dd, J=1.2 Hz, 4.8 Hz, 1H), 6.95 (brs, 1H), 6.84 (brs, 1H), 6.83 (s, 1H), 4.08-4.06 (m, 1H), 3.96-3.95 (m, 1H), 3.58-3.42 (m, 2H), 3.34-3.30 (m, 4H), 2.95-2.90 (m, 1H) 2.84-2.81 (m, 2H), 2.47-2.44 (m, 2H), 1.89-1.87 (m, 2H), 1.63-1.60 (m, 2H), 1.42-1.40 (m, 4H).

### Example 80: N-(5-(3-(((1,3-trans)-3-Hydroxycyclobutyl)carbamoyl)azetidin-1-yl)pentyl)-5-(thiophen-2-yl)isoxazole-3-carboxamide

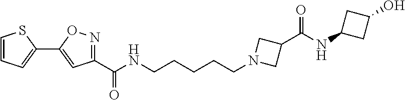 [SMILES: O=C(NCCCCCN1CC(C(=O)N[C@H]2C[C@H](O)C2)C1)c1cc(-c2cccs2)on1]

The title compound was prepared by using a procedure similar to that of Example 77 by replacing of 2-aminoacetonitrile with (1,3-trans)-3-aminocyclobutan-1-ol as a white solid. MS (ESI) m/z 433.1 [M+H]⁺. ¹H NMR (400 MHz, CDCl₃) δ ppm 7.55 (dd, J=1.2 Hz, 2.4 Hz, 1H), 7.51 (dd, J=1.2 Hz, 5.2 Hz, 1H), 7.15 (dd, J=1.2 Hz, 5.2 Hz, 1H), 6.92 (brs, 1H), 6.82 (s, 1H), 6.59 (brs, 1H), 4.54-4.44 (m, 2H), 3.48-3.43 (m, 2H), 3.38-3.36 (m, 2H), 3.29-3.26 (m, 2H), 3.02-2.98 (m, 1H), 2.44-2.34 (m, 4H), 2.28-2.24 (m, 2H), 1.51-1.50 (m, 2H) 1.42-1.38 (m, 4H).

### Example 81: N-(5-(3-((3-Hydroxypropyl)carbamoyl)azetidin-1-yl)pentyl)-5-(thiophen-2-yl)isoxazole-3-carboxamide

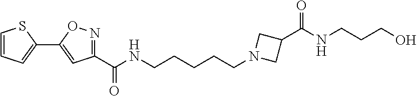 [SMILES: O=C(NCCCCCN1CC(C(=O)NCCCO)C1)c1cc(-c2cccs2)on1]

The title compound was prepared by using a procedure similar to that of Example 77 by replacing of 2-aminoacetonitrile with 3-aminopropan-1-ol as a light yellow solid. MS (ESI) m/z 421.1 [M+H]⁺. ¹H NMR (400 MHz, CD₃OD) δ ppm 7.69-7.66 (m, 2H), 7.20 (dd, J=4.0 Hz, 4.8 Hz, 1H), 6.90 (s, 1H), 3.58-3.54 (m, 2H), 3.53-3.49 (m, 2H), 3.32-3.31 (m, 2H), 3.30-3.29 (m, 5H), 2.50-2.46 (m, 2H), 1.71-1.61 (m, 4H), 1.40-1.39 (m, 4H).

### Example 82: N-(5-(3-((3-Hydroxycyclopentyl)carbamoyl)azetidin-1-yl)pentyl)-5-(thiophen-2-yl)isoxazole-3-carboxamide

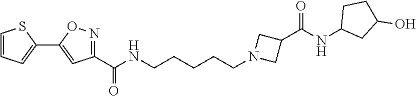 [SMILES: O=C(NCCCCCN1CC(C(=O)NC2CCC(O)C2)C1)c1cc(-c2cccs2)on1]

The title compound was prepared by using a procedure similar to that of Example 77 by replacing of 2-aminoacetonitrile with 3-aminocyclopentan-1-ol as a red solid. MS (ESI) m/z 447.2 [M+H]⁺. ¹H NMR (400 MHz, CD₃OD) δ ppm 7.71-7.68 (m, 2H), 7.24-7.22 (dd, J=3.6 Hz, 4.8 Hz, 1H), 6.92 (s, 1H), 4.34-4.13 (m, 2H), 3.59-3.31 (m, 2H), 3.40-3.38 (m, 2H), 3.30-3.29 (m, 3H), 2.55-2.51 (m, 2H), 2.27-2.15 (m, 1H), 2.00-1.96 (m, 1H), 1.67-1.55 (m, 5H), 1.44-1.41 (m, 5H).

### Example 83: N-(5-(3-((2-Hydroxycyclopentyl)carbamoyl)azetidin-1-yl)pentyl)-5-(thiophen-2-yl)isoxazole-3-carboxamide

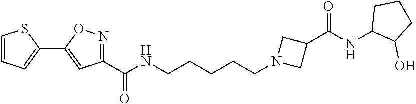 [SMILES: O=C(NCCCCCN1CC(C(=O)NC2CCCC2O)C1)c1cc(-c2cccs2)on1]

The title compound was prepared by using a procedure similar to that of Example 77 by replacing of 2-aminoacetonitrile with 2-aminocyclopentan-1-ol as a brown solid. MS (ESI) m/z 447.2 [M+H]⁺. ¹H NMR (400 MHz, CD₃OD) δ ppm 7.71-7.67 (m, 2H), 7.24-7.22 (dd, J=3.6 Hz, 5.2 Hz, 1H), 6.92 (s, 1H), 3.95-3.93 (m, 2H), 3.53-3.50 (m, 2H), 3.42-3.40 (m, 2H), 3.29-3.24 (m, 3H), 2.53-2.49 (m, 2H), 2.10-2.08 (m, 1H), 1.95-1.90 (m, 1H), 1.78-1.59 (m, 5H), 1.47-1.41 (m, 5H).

### Example 84: N-(5-(3-((2-Cyanoethyl)carbamoyl)azetidin-1-yl)pentyl)-5-(thiophen-2-yl)isoxazole-3-carboxamide

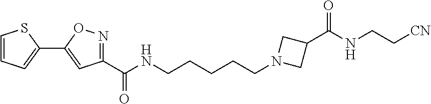 [SMILES: N#CCCNC(=O)C1CN(CCCCCNC(=O)c2cc(-c3cccs3)on2)C1]

The title compound was prepared by using a procedure similar to that of Example 77 by replacing of 2-aminoacetonitrile with 3-aminopropanenitrile as a white solid. MS (ESI) m/z 416.1 [M+H]⁺. ¹H NMR (400 MHz, CDCl₃) δ ppm 7.55 (dd, J=1.2 Hz, 4 Hz, 1H), 7.51 (dd, J=1.2 Hz, 5.2 Hz, 1H), 7.21 (brs, 1H), 7.15 (dd, J=5.2 Hz, 4 Hz, 1H), 6.97 (brs, 1H), 6.82 (s, 1H), 3.57-3.52 (m, 2H), 3.47-3.42 (m, 2H), 3.36-3.32 (m, 4H), 3.04 (m, 1H), 2.67-2.65 (m, 2H), 2.45-2.42 (m, 2H), 1.64-1.59 (m, 2H), 1.41-1.39 (m, 4H).

### Example 85: 5-(4-Fluorophenyl)-N-(5-(3-(methylcarbamoyl)azetidin-1-yl)pentyl)isoxazole-3-carboxamide

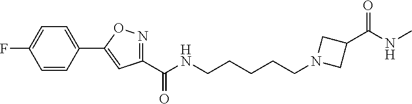 [SMILES: CNC(=O)C1CN(CCCCCNC(=O)c2cc(-c3ccc(F)cc3)on2)C1]

### Step 1: Preparation of 5-(4-Fluorophenyl)-N-(5-hydroxypentyl)isoxazole-3-carboxamide

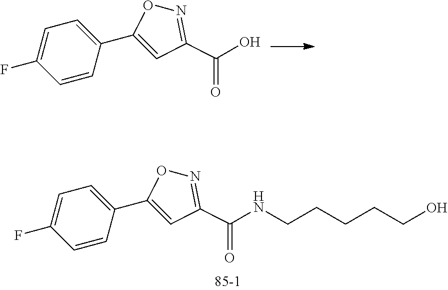 [SMILES: O=C(O)c1cc(-c2ccc(F)cc2)on1 ; O=C(NCCCCCO)c1cc(-c2ccc(F)cc2)on1]

The title compound was prepared by using a procedure similar to that of Intermediated B-1 by replacing of Intermediate A with 5-(4-fluorophenyl)isoxazole-3-carboxylic acid as a white solid. MS (ESI) m/z 293.0 [M+H]⁺.

### Step 2: Preparation of N-(5-Bromopentyl)-5-(4-fluorophenyl)isoxazole-3-carboxamide

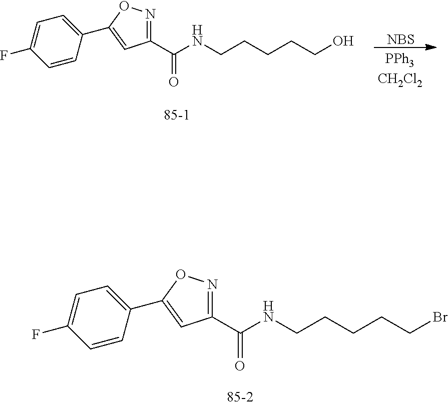

The title compound was prepared by using a procedure similar to that of 26-1 as an off-white solid. MS (ESI) m/z 355.0 [M+H]⁺.

### Step 3: Preparation of Methyl 1-(5-(5-(4-fluorophenyl)isoxazole-3-carboxamido)pentyl)azetidine-3-carboxylate

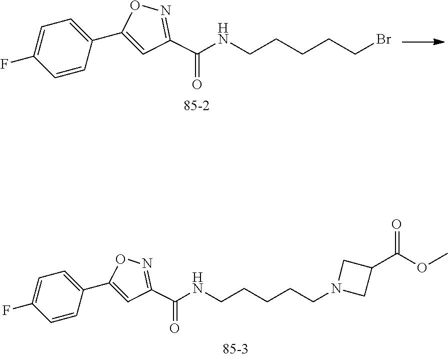 [SMILES: O=C(NCCCCCBr)c1cc(-c2ccc(F)cc2)on1 ; COC(=O)C1CN(CCCCCNC(=O)c2cc(-c3ccc(F)cc3)on2)C1]

The title compound was prepared by using a procedure similar to that of compound 77-1 as a white solid. MS (ESI) m/z 390.2 [M+Na]⁺.

### Step 4: Preparation of 1-(5-(5-(4-Fluorophenyl)isoxazole-3-carboxamido)pentyl)azetidine-3-carboxylic Acid

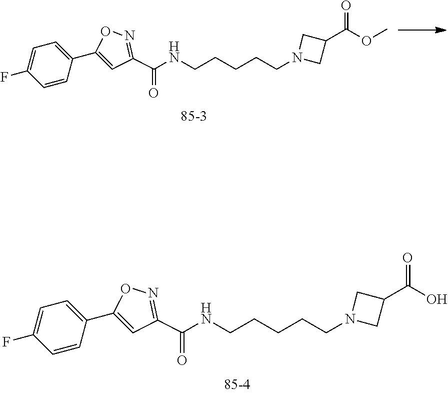 [SMILES: COC(=O)C1CN(CCCCCNC(=O)c2cc(-c3ccc(F)cc3)on2)C1 ; O=C(NCCCCCN1CC(C(=O)O)C1)c1cc(-c2ccc(F)cc2)on1]

The title compound was prepared by using a procedure similar to that of compound 77-2.

### Step 5: Preparation of 5-(4-Fluorophenyl)-N-(5-(3-(methylcarbamoyl)azetidin-1-yl)pentyl)isoxazole-3-carboxamide

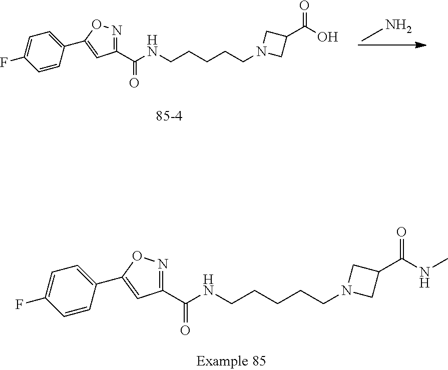

The title compound was prepared by using a procedure similar to that of Example 77 by replacing of 2-aminoacetonitrile with methylamine as a white solid. MS (ESI) m/z 389.0 [M+H]⁺. ¹H NMR (400 MHz, CD₃OD) δ ppm 7.94-7.91 (m, 2H), 7.27 (t, J=8.8 Hz, 2H), 7.04 (s, 1H), 3.51-3.50 (m, 2H), 3.41-3.39 (m, 2H), 3.24-3.23 (m, 3H), 2.71 (s, 3H), 2.48-2.46 (m, 2H), 1.63-1.62 (m, 2H), 1.41-1.39 (m, 4H).

### Example 86: N-(5-(3-(Ethylcarbamoyl)azetidin-1-yl)pentyl)-5-(4-fluorophenyl)isoxazole-3-carboxamide

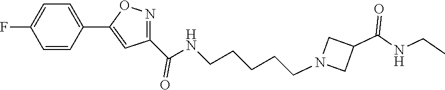 [SMILES: CCNC(=O)C1CN(CCCCCNC(=O)c2cc(-c3ccc(F)cc3)on2)C1]

The title compound was prepared by using a procedure similar to that of Example 85 by replacing of methylamine with ethylamine as a white solid. MS (ESI) m/z 403.2 [M+H]⁺.

¹H NMR (400 MHz, CD₃OD) δ ppm 7.97-7.93 (m, 2H), 7.30 (t, J=8.8 Hz, 2H), 7.07 (s, 1H), 3.55-3.54 (m, 2H), 3.42-3.41 (m, 2H), 3.30-3.25 (m, 3H), 3.22-3.20 (m, 2H), 2.52-2.50 (m, 2H), 1.67-1.64 (m, 2H), 1.43-1.42 (m, 4H), 1.12 (t, J=7.2 Hz, 3H).

### Example 87: N-(5-(3-((2-Cyanoethyl)carbamoyl)azetidin-1-yl)pentyl)-5-(4-fluorophenyl)isoxazole-3-carboxamide

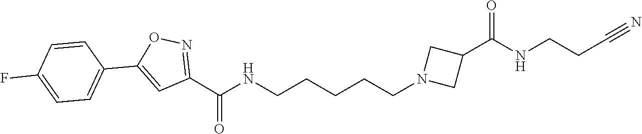 [SMILES: N#CCCNC(=O)C1CN(CCCCCNC(=O)c2cc(-c3ccc(F)cc3)on2)C1]

The title compound was prepared by using a procedure similar to that of Example 85 by replacing of methylamine with 3-aminopropanenitrile as a white solid. MS (ESI) m/z 428.2 [M+H]⁺. ¹H NMR (400 MHz, CD₃OD) δ ppm 7.96-7.93 (m, 2H), 7.30 (t, J=8.8 Hz, 2H), 7.07 (s, 1H), 3.55-3.54 (m, 2H), 3.44-3.39 (m, 4H), 3.29-3.28 (m, 3H), 2.70-2.67 (m, 2H), 2.51-2.49 (m, 2H), 1.67-1.64 (m, 2H), 1.43-1.41 (m, 4H).

### Example 88: N-(5-(3-((Cyanomethyl)carbamoyl)azetidin-1-yl)pentyl)-5-(4-fluorophenyl)isoxazole-3-carboxamide

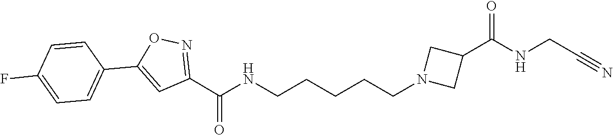 [SMILES: N#CCNC(=O)C1CN(CCCCCNC(=O)c2cc(-c3ccc(F)cc3)on2)C1]

The title compound was prepared by using a procedure similar to that of Example 85 by replacing of methylamine with 2-aminoacetonitrile as a yellow solid. MS (ESI) m/z 436.3 [M+Na]⁺. ¹H NMR (400 MHz, CD₃OD) δ ppm 7.94-7.90 (m, 2H), 7.27 (t, J=8.8 Hz, 2H), 7.04 (s, 1H), 4.14 (s, 2H), 3.54-3.50 (m, 2H), 3.40-3.37 (m, 2H), 3.30-3.29 (m, 3H), 2.51-2.48 (m, 2H), 1.65-1.61 (m, 2H), 1.41-1.39 (m, 4H).

### Example 89: 5-(4-Fluorophenyl)-N-(5-(3-(((1,3-trans)-3-hydroxycyclobutyl)carbamoyl)azetidin-1-yl)pentyl)isoxazole-3-carboxamide

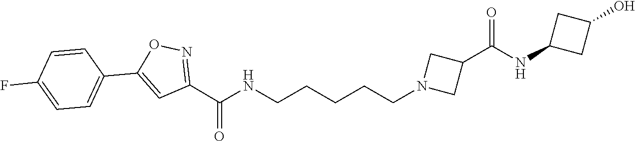 [SMILES: O=C(NCCCCCN1CC(C(=O)N[C@H]2C[C@H](O)C2)C1)c1cc(-c2ccc(F)cc2)on1]

The title compound was prepared by using a procedure similar to that of Example 85 by replacing of methylamine with (1,3-trans)-3-aminocyclobutan-1-ol hydrochloride as a white solid. MS (ESI) m/z 445.3 [M+H]⁺. ¹H NMR (400 MHz, CD₃OD) δ ppm 7.93-7.90 (m, 2H), 7.26 (t, J=8.8 Hz, 2H), 7.04 (s, 1H), 4.35-4.28 (m, 2H), 3.54-3.53 (m, 2H), 3.42-3.39 (m, 2H), 3.27-3.26 (m, 3H), 2.48-2.46 (m, 2H), 2.24-2.20 (m, 4H), 1.62-1.60 (m, 2H), 1.40-1.38 (m, 4H).

### Example 90: 5-(4-Fluorophenyl)-N-(5-(3-((2-hydroxyethyl)carbamoyl)azetidin-1-yl)pentyl)isoxazole-3-carboxamide

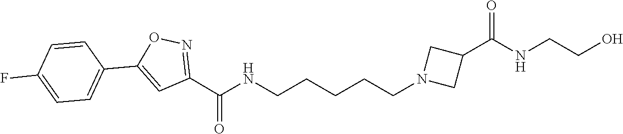 [SMILES: O=C(NCCCCCN1CC(C(=O)NCCO)C1)c1cc(-c2ccc(F)cc2)on1]

The title compound was prepared by using a procedure similar to that of Example 85 by replacing of methylamine with 2-aminoethan-1-ol as an off-white solid. MS (ESI) m/z 419.2 [M+H]⁺. ¹H NMR (400 MHz, CD₃OD) δ ppm 7.93-7.90 (m, 2H), 7.26 (t, J=8.8 Hz, 2H), 7.04 (s, 1H), 3.58-3.56 (m, 4H), 3.41-3.32 (m, 5H), 3.29-3.28 (m, 2H), 2.55-2.52 (m, 2H), 1.64-1.61 (m, 2H), 1.41-1.39 (m, 4H).

### Example 91: 5-(4-Fluorophenyl)-N-(5-(3-(((1S,2S)-2-hydroxycyclopentyl)carbamoyl)azetidin-1-yl)pentyl)isoxazole-3-carboxamide

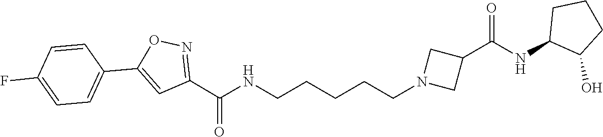 [SMILES: O=C(NCCCCCN1CC(C(=O)N[C@H]2CCC[C@@H]2O)C1)c1cc(-c2ccc(F)cc2)on1]

The title compound was prepared by using a procedure similar to that of Example 85 by replacing of methylamine with (1S,2S)-2-aminocyclopentan-1-ol hydrochloride as an off-white solid. MS (ESI) m/z 459.3 [M+H]⁺. ¹H NMR (400 MHz, CD₃OD) δ ppm 7.97-7.93 (m, 2H), 7.30 (t, J=8.8 Hz, 2H), 7.08 (s, 1H), 4.27-4.20 (m, 4H), 3.98-3.93 (m, 2H), 3.58-3.55 (m, 1H), 3.46-3.42 (m, 2H), 3.23-3.19 (m, 2H), 2.18-2.10 (m, 1H), 1.98-1.94 (m, 1H), 1.78-1.60 (m, 7H), 1.48-1.45 (m, 3H).

### Example 92: 5-(4-Fluorophenyl)-N-(5-(3-(((1,3-cis)-3-hydroxycyclobutyl)carbamoyl)azetidin-1-yl)pentyl)isoxazole-3-carboxamide

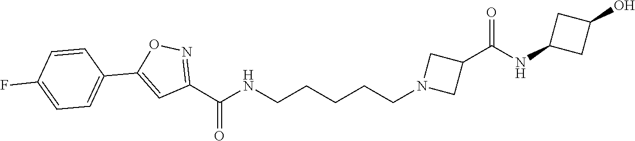 [SMILES: O=C(NCCCCCN1CC(C(=O)N[C@H]2C[C@@H](O)C2)C1)c1cc(-c2ccc(F)cc2)on1]

The title compound was prepared by using a procedure similar to that of Example 85 by replacing of methylamine with (1,3-cis)-3-aminocyclobutan-1-ol hydrochloride as an off-white solid. MS (ESI) m/z 445.2 [M+H]⁺. ¹H NMR (400 MHz, CDCl₃) δ ppm 7.81-7.78 (m, 2H), 7.19 (t, J=8.8 Hz, 2H), 7.00-6.95 (m, 1H), 6.93 (s, 1H), 6.84-6.78 (m, 1H), 4.10-3.97 (m, 2H), 3.51-3.46 (m, 2H) 3.30-3.28 (m, 4H), 2.85-2.84 (m, 1H), 2.82-2.81 (m, 2H), 2.72-2.71 (m, 1H), 2.45-2.42 (m, 2H), 1.90-1.87 (m, 2H), 1.66-1.63 (m, 2H), 1.45-1.37 (m, 4H).

### Example 93: N-(5-(3-Acetylazetidin-1-yl)pentyl)-5-(thiophen-2-yl)isoxazole-3-carboxamide

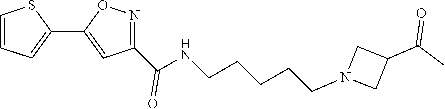 [SMILES: CC(=O)C1CN(CCCCCNC(=O)c2cc(-c3cccs3)on2)C1]

### Step 1: Preparation of N-(5-(3-(Methoxy(methyl)carbamoyl)azetidin-1-yl)pentyl)-5-(thiophen-2-yl)isoxazole-3-carboxamide

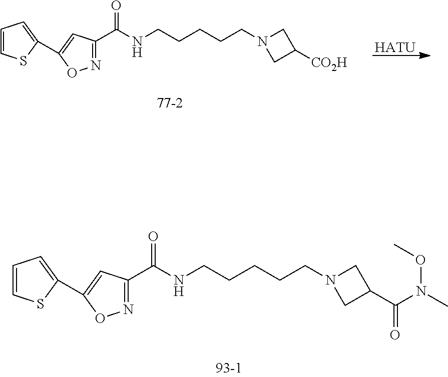 [SMILES: O=C(NCCCCCN1CC(C(=O)O)C1)c1cc(-c2cccs2)on1 ; "HATU" ; CON(C)C(=O)C1CN(CCCCCNC(=O)c2cc(-c3cccs3)on2)C1]

To a solution of compound 77-2 (0.2 g, 0.55 mmol, 1.0 eq) in DMF (2 mL) was added N,O-dimethylhydroxylamine hydrochloride (161 mg, 1.65 mmol, 3.0 eq), HATU (419 mg, 1.1 mmol, 2.0 eq) and DIEA (356 mg, 2.75 mmol, 5.0 eq). The mixture was stirred at 25° C. for 14 hours. The mixture was diluted with water (10 mL), the aqueous phase was extracted with DCM (3*10 mL). The combined organic phase was dried over Na₂SO₄, filtered and the filtrate was concentrated. The residue was purified by silica gel chromatography eluting with DCM/MeOH from 30/1 to 10/1 to afford the title compound (0.2 g, 89.4% yield) as a light yellow solid. MS (ESI) m/z 407.1 [M+H]⁺.

### Step 2: Preparation of N-(5-(3-Acetylazetidin-1-yl)pentyl)-5-(thiophen-2-yl)isoxazole-3-carboxamide

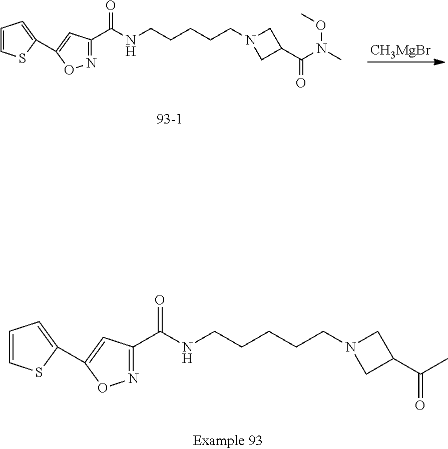

To a solution of 93-1 (0.15 g, 0.369 mmol, 1 eq) in THF (2 mL) was added CH₃MgBr (1.23 mL, 3.69 mmol, 10 eq) at 0° C. The mixture was stirred at 0° C. for 4 hours. The reaction mixture was poured into 10 mL of saturated NH₄Cl aqueous solution. The aqueous phase was extracted with EtOAc (3*10 mL). The combined organic phase was dried over Na₂SO₄, and filtered. The filtrate was concentrated under reduced pressure. The residue was purified by basic preparative HPLC (Kromasil 150*25 mm*10 um, gradient: 25-55% B (A=0.05% ammonia hydroxide/water, B=CH₃CN), flow rate: 30 mL/min) to afford the title compound (14.5 mg, 10.8% yield) as white solid. MS (ESI) m/z 362.2 [M+H]⁺. ¹H NMR (400 MHz, DMSO-d₆) δ ppm 8.78 (t, J=5.2 Hz, 1H), 7.86 (dd, J=1.2 Hz, 5.2 Hz, 1H), 7.79 (d, J=2.8 Hz, 1H), 7.26 (dd, J=4.0 Hz, 5.2 Hz, 1H), 7.16 (s, 1H), 3.30-3.29 (m, 3H), 3.23-3.21 (m, 2H), 3.07-3.06 (m, 2H), 2.28-2.26 (m, 2H), 2.06 (s, 3H), 1.51-1.47 (m, 2H), 1.26-1.24 (m, 4H).

### Example 94: N-(5-(5,6-dihydroimidazo[1,5-a]pyrazin-7(8H)-yl)pentyl)-5-(thiophen-2-yl)isoxazole-3-carboxamide

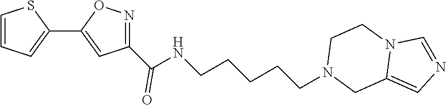 [SMILES: O=C(NCCCCCN1CCn2cncc2C1)c1cc(-c2cccs2)on1]

To a solution of intermediate B (150 mg, 0.54 mmol, 1.0 eq) in CH2ClCH2Cl (10 mL) were added imidazo[1,5-a]pyrazine,5,6,7,8-tetrahydro-(9Cl) (132.7 g, 1.07 mmol, 2.0 eq), NaBH(OAc)3 (685.3 mg, 3.24 mmol, 6.0 eq), acetic acid (97.1 mg, 1.62 mmol, 3.0 eq). Then the mixture was stirred at 15° C. for 12 hours. The mixture was quenched with water (10 mL). The mixture was extracted with DCM. The combined organic phase was concentrated to obtain the crude product which was purified by preparative HPLC (column: Xtimate C18 150*25 mm*5 um, gradient: 33-63% B (A=0.05% ammonia hydroxide/water, B=acetonitrile) to give the title compound (95 mg, 45.7% yield) as a light yellow solid. MS (ESI) m/z 386.0 [M+H]⁺. ¹H NMR (400 MHz, DMSO-d₆) δ 8.80 (t, J=5.6 Hz, 1H), 7.86 (dd, J=5.2, 0.8 Hz, 1H), 7.78 (dd, J=4.0, 1.2 Hz, 1H), 7.49 (s, 1H), 7.27-7.25 (m, 1H), 7.16 (s, 1H), 6.61 (s, 1H), 3.97 (t, J=5.6 Hz, 2H), 3.53 (s, 2H), 3.28-3.23 (m, 2H), 2.73 (t, J=5.6 Hz, 2H), 2.45 (t, J=6.8 Hz, 2H), 1.58-1.48 (m, 4H), 1.36-1.30 (m, 2H).

### Example 95: N-(5-(3-(1H-imidazol-2-yl)azetidin-1-yl)pentyl)-5-(thiophen-2-yl)isoxazole-3-carboxamide

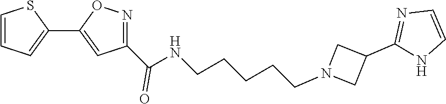 [SMILES: O=C(NCCCCCN1CC(c2ncc[nH]2)C1)c1cc(-c2cccs2)on1]

### Step 1: Preparation of tert-butyl 3-(1H-imidazol-2-yl)azetidine-1-carboxylate

Ammonia gas was bubbled through a mixture of tert-butyl 3-formylazetidine-1-carboxylate (1.0 g, 5.4 mmol, 1.0 eq) and glyoxal (10.9 g, 40 wt % in water, 75.59 mmol, 14 eq.) at 0° C. for 10 min, until the weight of the solution increase 1.84 g (about 107.98 mmol of NH₃). The mixture was allowed to warm to 26° C. and stirred for 14 hours. The aqueous layers were extracted with CH₂Cl₂. The combined organic phase was dried over Na₂SO₄, filtered and concentrated under reduced pressure. The residue was purified by silica gel chromatography eluting with 70% EtOAc in hexane to afford the title compound (0.51 g, 42% yield) as a light yellow solid. MS (ESI) m/z 224.0 [M+H]⁺.

¹H NMR (400 MHz, CDCl₃) δ 7.01 (s, 2H), 4.28 (t, J=8.8 Hz, 2H), 4.16-4.11 (m, 2H), 3.88-3.86 (m, 1H), 1.44 (s, 9H).

### Step 2: Preparation of 2-(azetidin-3-yl)-1H-imidazole

To a solution of tert-butyl 3-(1H-imidazol-2-yl)azetidine-1-carboxylate (0.3 g, 1.34 mmol, 1.0 eq) in CH₂Cl₂ (2 mL) was added TFA (0.5 mL). The mixture was stirred for 48 hours at 27° C. The volatile was removed under reduced pressure to afford the title compound (0.5 g, 100% yield, 94.3% wt) as a light yellow oil which was used without further purification.

### Step 3: Preparation of N-(5-(3-(1H-imidazol-2-yl)azetidin-1-yl)pentyl)-5-(thiophen-2-yl)isoxazole-3-carboxamide

The title compound was prepared by using a procedure similar to that of Example 94 by replacing of imidazo[1,5-a]pyrazine,5,6,7,8-tetrahydro-(9Cl) with 2-(azetidin-3-yl)-1H-imidazole in 13% yield as a white solid. MS (ESI) m/z 386.1 [M+H]⁺. ¹H NMR (400 MHz, CD₃OD) δ 7.71-7.68 (m, 2H), 7.23 (dd, J=4 Hz, 5.2 Hz, 1H), 6.79 (s, 2H), 6.92 (s, 1H), 3.79-3.76 (m, 3H), 3.41-3.33 (m, 4H), 2.62-2.58 (m, 2H), 1.69-1.65 (m, 2H), 1.48-1.43 (m, 4H).

### Pharmaceutical Compositions and Combinations

The compounds of the present disclosure are typically used as a pharmaceutical composition (e.g., a compound of the present disclosure and at least one pharmaceutically acceptable carrier). A “pharmaceutically acceptable carrier (diluent or excipient)” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

In one aspect, the present disclosure provides a pharmaceutical composition comprising a compound of the present disclosure, or a pharmaceutically acceptable salt thereof, and a pharmaceutically acceptable carrier. In a further embodiment, the composition comprises at least two pharmaceutically acceptable carriers, such as those described herein. For purposes of the present disclosure, unless designated otherwise, solvates and hydrates are generally considered compositions. Preferably, pharmaceutically acceptable carriers are sterile. The pharmaceutical composition can be formulated for particular routes of administration such as oral administration, parenteral administration, and rectal administration, etc. In addition, the pharmaceutical compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Tablets may be either film coated or enteric coated according to methods known in the art.

Suitable compositions for oral administration include an effective amount of a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present disclosure further provides anhydrous pharmaceutical compositions and dosage forms comprising the compounds of the present disclosure as active ingredients, since water may facilitate the degradation of certain compounds.

Anhydrous pharmaceutical compositions and dosage forms of the disclosure can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The present disclosure further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present disclosure is typically formulated into pharmaceutical dosage forms to provide an easily controllable dosage of the drug and to give the patient an elegant and easily handleable product. The dosage regimen for the compounds of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. Compounds of this disclosure may be administered in a single daily dose, or the total daily dosage may be administered in divided doses of two, three, or four times daily.

The present disclosure further provides pharmaceutical compositions which can be delivered locally to the subject, including administration in the form of solid, semi-solid, liquid, gels, and microspheres, etc., into the outer ear, middle ear or inner ear. Compositions of the present disclosure can be administered by a number of methods sufficient to deliver the composition to the inner ear. Such methods include, but are not limited to, auricular administration (e.g., by transtympanic wicks or catheters), intraauricular administration, intratympanic administration, intracochlear administration, intravestibular administration and intralabyrinth administration.

As used herein, the term “auricular administration” refers to a method of using a catheter or wick device to administer a composition across the tympanic membrane to the inner ear of the subject. To facilitate insertion of the wick or catheter, the tympanic membrane may be pierced using a suitably sized syringe. The devices could also be inserted using any other methods known to those of skill in the art, e.g., surgical implantation of the device. In particular embodiments, the wick or catheter device may be a stand alone device, meaning that it is inserted into the ear of the subject and then the composition is controllably released to the inner ear. In other particular embodiments, the wick or catheter device may be attached or coupled to a pump or other device that allows for the administration of additional compositions. The pump may be automatically programmed to deliver dosage units or may be controlled by the subject or medical professional.

As used herein, the term “Intraauricular” administration refers to administration of a composition to the outer, the middle or inner ear of a subject by directly injecting the composition. “intratympanic” administration refers to the injection or perfusion of a composition across the tympanic membrane into the middle ear, such that the composition may diffuse across the round window membrance into the inner ear. “Intracochlear” administration refers to direct delivery of a composition into the cochlea. “Intravestibular” administration refers to direct delivery of a composition into the vestibular organs. “Intralabyrinth” administration refers to direct delivery of a composition into the inner ear fluid compartment to expose the inner ear including the semicircular canals, the vestibule and cochlea to the composition.

In one embodiment, a syringe and needle apparatus is used to administer compositions to a subject using auricular administration. A suitably sized needle is used to pierce the tympanic membrane and a wick or catheter comprising the composition is inserted through the pierced tympanic membrane and into the middle ear of the subject. The device may be inserted such that it is in contact with the round window or immediately adjacent to the round window. Exemplary devices used for auricular administration include, but are not limited to, transtympanic wicks, transtympanic catheters, transtympanic pumps, round window microcatheters (small catheters that deliver medicine to the round window), and Silverstein Microwicks™ (small tube with a “wick” through the tube to the round window, allowing regulation by subject or medical professional).

In another embodiment, a syringe and needle apparatus is used to administer compositions to a subject into the middle and/or inner ear. The formulation may be administered directly onto the round window membrane via intratympanic injection, or may be administered directly to the cochlea via intracochlear injection, or directly to the vestibular organs via intravestibular injection, or directly to the semicircular canals, the vestibule and the cochlea via intralabyrinth injection.

In still another embodiment, the delivery device can be an apparatus designed for administration of compositions to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. Patent Application Publication 2007/0167918, which is incorporated herein by reference for such disclosure, further describes a combined otic aspirator and medication dispenser for transtympanic fluid sampling and medicament application.

In one embodiment, the compositions may be locally administered to the subject. In another embodiment, the compositions may be administered to the subject by auricular administration. In still another embodiment, the compositions may be administered to the subject by intraauricular administration. In still another embodiment, the compositions may be administered to the subject by intratympanic administration. In still another embodiment, the compositions may be administered to the subject by intracochlear administration. In still another embodiment, the compositions may be administered to the subject by intravestibular administration. In still another embodiment, the compositions may be administered to the subject by intralabyrinth administration.

In one embodiment, the compositions comprise one or more components that enhance the availability of the active ingredients of the composition to the cochlea, and/or provide extended or immediate release of active ingredients of the composition to the inner ear. In one embodiment, the one or more components are pharmaceutically acceptable carriers.

In another embodiment, the compositions comprise one or more pharmaceutically acceptable carriers that will facilitate the delivery of the composition across biological barriers that separate the middle and inner ear, e.g., the round window, thereby efficiently delivery a therapeutically effective amount of the composition to the inner ear. Efficient delivery to the cochlea, Organ of Corti, vestibular organs, and/or the inner ear perilymph or endolymph fluid space is desired because these tissues/organs host the supporting cells that promote sensory hair cell regeneration when treated or contacted with compositions of the present disclosure.

Intratympanic delivery to the inner ear can be performed via the injection or perfusion of the composition to the middle ear with the aim of the composition diffusion through the round window membrane into the inner ear. Delivery systems suitable for the intratympanic administration are well known and can be found in, for example, Liu et al., Acta Pharmaceutica Sinica B 2013; 3(2):86-96; Kechai et al., International Journal of Pharmaceutics 2015; 494: 83-101; and Ayoob et al., Expert Opinion on Drug Delivery, 2015; 12(3): 465-479.

In certain instances, it may be advantageous to administer the compound of the present disclosure in combination with one or more therapeutically active agents, for example, those therapeutically active agents related to relevant hair cell development/regeneration pathways, including but not limited to, Notch signaling, FGF signaling, Wnt Signaling, Shh signaling, cell cycle/stem cell aging, miRNA and epigenetic regulations.

The term “combination therapy” refers to the administration of two or more therapeutic agents to treat a therapeutic disease, disorder or condition described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. The compound of the present disclosure and additional therapeutic agents can be administered via the same administration route or via different administration routes. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the diseases, conditions or disorders described herein.

In one embodiment, the present disclosure provides pharmaceutical compositions comprising at least one compound of the present disclosure or a pharmaceutically acceptable salt thereof together with a pharmaceutically acceptable carrier suitable for administration to a human or animal subject, either alone or together with one or more other therapeutically active agents related to those relevant hair cell development/regeneration pathways as described in the above.

In another embodiment, the present disclosure provides methods of treating a human or animal subject for hearing loss or balance disorder, comprising administering to the subject a therapeutically effective amount of a compound of the present disclosure or a pharmaceutically acceptable salt thereof, either alone or in combination with one or more other therapeutically active agents related to those relevant hair cell development/regeneration pathways as described in the above.

In particular, compositions will either be formulated together as a combination therapeutic or administered separately.

In combination therapy for treatment of hearing loss or balance disorder, the compound of the present disclosure and other therapeutically active agent(s) may be administered simultaneously, concurrently or sequentially with no specific time limits, wherein such administration provides therapeutically effective levels of the two compounds in the body of the subject.

In a preferred embodiment, the compound of the present disclosure and the other therapeutically active agent(s) is generally administered sequentially in any order by infusion, orally or locally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present disclosure and other therapeutically active agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present disclosure, a kit comprising two or more separate pharmaceutical compositions, at least one of which contains a compound of the present disclosure is provided. In one embodiment, the kit comprises means for separately retaining said compositions, such as a container, divided bottle, or divided foil packet. An example of such a kit is a blister pack, as typically used for the packaging of tablets, capsules and the like.

The kit of the present disclosure may be used for administering different dosage forms, for example, oral and parenteral, for administering the separate compositions at different dosage intervals, or for titrating the separate compositions against one another. To assist compliance, the kit of the present disclosure typically comprises directions for administration.

In the combination therapies of the present disclosure, the compound of the present disclosure and the other therapeutic agent may be manufactured and/or formulated by the same or different manufacturers. Moreover, the compound of the present disclosure and the other therapeutic (or pharmaceutical agent) may be brought together into a combination therapy: (i) prior to release of the combination product to physicians (e.g. in the case of a kit comprising the compound of the present disclosure and the other therapeutic agent); (ii) by the physician themselves (or under the guidance of the physician) shortly before administration; (iii) in the patient themselves, e.g. during sequential administration of the compound of the present disclosure and the other therapeutic agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition or combination of the present disclosure can be in unit dosage of about 1-10000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties may be demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg

### Pharmacology and Utility

The present disclosure relates generally to compounds, compositions and methods for treating hearing loss and balance disorder associated with the damage or loss of sensory hair cells in the inner ear by increasing, promoting, stimulating or inducing the regeneration of sensory hair cells in the inner ear. Therefore, a brief review of the anatomy of the ear may be helpful in understanding the present disclosure.

The anatomy of the ear is well known to those of ordinary skill in the art (see, e.g., Gray's Anatomy, Revised American Edition (1977), pages 859-867). The ear is generally divided into three portions: the outer ear, middle ear, and inner ear. The outer ear is composed of auricle (the pinna), the auditory canal, and the outward facing portion of the tympanic membrane (ear drum). The function of the outer ear, in part, is to collect and direct sound waves through the auditory canal towards the tympanic membrane and the middle ear.

The middle ear is an air-filled cavity that includes the tympanic cavity, three ear bones (auditory ossicles): the malleus, the incus and the stapes, oval window and round window, which connects the middle ear with the inner ear. The auditory ossicles are arranged to provide a mechanical linkage between the tympanic membrane and the oval window to the fluid-filled inner ear, where sound is transformed and transduced to the inner ear for further processing.

The inner ear contains sensory organs for hearing and balance. The cochlea senses sound; the balance organ includes semicircular canals, which sense angular acceleration; and the otolithic organs (utricle and saccule), which sense linear acceleration. The round window that connects the cochlea to the middle ear. In each of these sensory portions, specialized sensory hair cells are arrayed upon one or more layers of inner ear supporting cells. Supporting cells underlie, at least partially surround, and physically support sensory hair cells within the inner ear. The stereocilia on the sensory hair cells are physically deflected in response to sound or motion, and their deflection is transmitted to nerves which send nerve impulses to the brain for processing and interpretation.

In particular, the cochlea includes the Organ of Corti which is primarily responsible for sensing sound. The Organ of Corti includes a basilar membrane upon which are located a variety of supporting cells, including border cells, inner pillar cells, outer pillar cells, inner phalangeal cells, Dieter's cells and Hensen's cells. Supporting cells surround and seperate inner hair cells and outer hair cells. The tectorial membrane is disposed above inner hair cells and outer hair cells.

Hearing loss and balance disorders are mainly caused by damage or loss of the sensory hair cells in the cochlea. In mammals, loss or damage to sensory hair cells results in permanent hearing loss or balance disorders, because they are generated only during embryonic development and do not spontaneously regenerate upon damage or cell loss during one's life time. It is widely accepted that although cells capable of generating sensory hair cells are present in the inner ear, natural sensory hair cell regeneration in the inner ear is low (Li et al., Trends Mol. Med., 10, 309-315 (2004); Li et al., Nat. Med., 9, 1293-1299 (2003); Rask-Andersen et al., Hear. Res., 203, 180-191 (2005)). As a result, lost or damaged sensory hair cells may not be adequately replaced by natural physiological processes (e.g., cell differentiation) and a loss of hair cells occurs. In many individuals, such sensory hair cell loss can result in, e.g., sensorineural hearing loss and balance disorders. Therefore, therapeutic strategies that increase the number of sensory hair cells in the inner ear will benefit a patient with sensory hair cell loss or damage.

Sensory hair cell fate determination in the inner ear is controlled by specific genes and pathways. Atonal protein homologue 1 (Atoh1 or atonal) is the master regulator of inner ear hair cell development and regeneration. The importance of Atoh1 in hair cell genesis is well documented. For example, Math1 (Atoh1 homolog in mouse) is required for hair cell development and the differentiation of inner ear progenitor cells to inner ear support cells and/or sensory hair cells (Bermingham et al., Science, 284:1837-1841, 1999). In addition, adenovirus mediated Math1 overexpression in the endolymph of the mature guinea pig results in the differentiation of non-sensory cells in the mature cochlea into immature hair cells (Kawamoto et al., J. Neurosci., 23:4395-4400, 2003). The implications of these studies are twofold. First, they demonstrate that non-sensory cells of the mature cochlear retain the ability to differentiate into sensory cells, e.g., sensory hair cells. Second, they demonstrate that Math1 overexpression is necessary and sufficient to direct supporting cells transdifferentiation into hair cells. A later study furthered these findings by demonstrating that adenovirus mediated Atoh1 overexpression induces sensory hair cell regeneration and substantially improves hearing thresholds in an experimentally deafened animal model (Izumikawa et al., Nat. Med., 11:271-276, 2005).

This suggests that although the mammalian cochlear sensory epithelium has lost the ability to spontaneously regenerate, the molecular activity required for inducing hair cell fate is still present and functional in mature supporting cells. These findings also suggest that activation of endogenous Atoh1 expression by pharmacological intervention could be an effective approach to stimulate sensory hair cell regeneration for treating hearing loss and balance disorders.

The present disclosure provides compounds, compositions and methods which are capable of increasing Atoh1 expression and/or activity in a subject. The present disclosure also provides compounds, compositions and methods which can increase or promote sensory hair cell regeneration. The present disclosure also provides compounds, compositions and methods which can increase the number of sensory hair cells in the inner ear of the subject. Consequently, the compounds, compositions and methods described herein can be used to treat hearing loss and/or balance disorders that result from the damage or loss of sensory hair cells in a subject.

The compounds of present disclosure in free form or in pharmaceutically acceptable salt form, exhibit valuable pharmacological properties, which can be demonstrated at least by using any one of the following test procedures. Compounds of the present disclosure were assessed for their ability to increase the Atoh1 expression in mouse cerebellar neural precursor cells. The ability of compounds of the present disclosure to induce new hair cell formation was assessed in ex vivo hair cell induction assay using 6-day-postnatal mouse cochlea explants with hair cell damage.

### Atoh1 Induction Assay in Mouse Cerebellar Neural Precursor Cells (NPCs)

Atoh1 induction assay was conducted with in vitro cultured cerebellar neural precursor cells isolated from neonatal transgenic Atoh1-GFP mice. Atoh1 expression is mainly regulated by the enhancer, and the nuclear GFP was driven by the cloned enhancer sequence at 3′ of Atoh1 which had high conservation among mammalians. So Atoh1 induction could be reflected by GFP activation in cerebellar neural precursor cells (Helms et al., Development 2000; 127: 1185-1196; Lumpkin et al., Gene Expression Patterns 2003; 3: 389-395). Postnatal 3 days pups were dissected for cerebellum tissue isolation. The cerebellum tissue was cut into small pieces, and dissociated with 0.05% Trypsin for about 10 minutes at 37° C., and then filtered with a 70 uM cell strainer. The cells were cultured as neuropsheres for the first 2 days in ultra-low attachment dish/well-plate with DMEM/F12+1% N2 &2% B27 with 1% P/S, 20 ng/ml rhFGF2 and 20 ng/ml rhEGF (R&D Systems). Then the spheres were plated to the matrigel (1:30 diluted in DMEM/F12)-coated tissue culture dish for monolayer culture. After 4.5-5.5 days culture in vitro (DIV), cells were dissociated with 0.05% trypsin into single cells, and frozen after cell number calculation.

The cerebellar neural precursor cells (NPCs) were re-thawed from stock and cultured for another 2 days before used for Atoh1 induction assay. On the first day of assay, NPCs were seeded into matrigel-coated 384 well plates (Black view-plate, PE) at 2500 cells/well. After over-night culture, the NPCs were treated with representative compounds of the present disclosure with 1:2 serial dilutions for 10 doses, from 50 μM to 200 nM, with DMSO as negative control. After 72 hours treatment without medium change, the cells were fixed with 4% formalin for staining. Assay plates were stained with GFP antibody (Abcam, #13970, 1:1000) to amplify endogenous GFP signal and then read by Cellomics. The GFP average intensity in cell nuclie which is defined by DAPI staining for the tested compounds were calculated and compared to DMSO control, and the difference is expressed in a fold difference format according to the equation of (the GFP average intensity of the tested compound/(the DMSO control). The maximum fold difference of each tested compound over the DMSO control is described in below Table 2 (see the column with the title “fold difference”). Note the value of the DMSO control is 1 in the equation, and any fold difference more than 5 is considered as a significant difference As shown in Table 2, all of the tested compounds of the present disclosure have demonstrated significant fold difference in terms of GFP average intensity over the DMSO control. Therefore, all of the tested compounds were active for the activation of Atoh1 and significantly increase the Atoh1 expression.

TABLE 2

| Example No. | fold difference |
|---|---|
| DMSO | 1.0 |
| 1 | 22.4 |
| 2 | 35.4 |
| 3 | 21.0 |
| 4 | 30.7 |
| 5 | 32.5 |
| 6 | 9.0 |
| 7 | 31.0 |
| 8 | 30.9 |
| 10 | 17.6 |
| 11 | 12.4 |
| 12 | 21.1 |
| 13 | 23.0 |
| 14 | 29.1 |
| 15 | 15.3 |
| 16 | 25.3 |
| 17 | 34.6 |
| 18 | 17.6 |
| 19 | 31.7 |
| 20 | 29.4 |
| 21 | 13.0 |
| 22 | 25.5 |
| 23 | 25.7 |
| 24 | 24.0 |
| 25 | 10.1 |
| 26 | 7.1 |
| 27 | 33.7 |
| 28 | 28.6 |
| 29 | 20.1 |
| 30 | 13.4 |
| 31 | 16.0 |
| 32 | 26.8 |
| 33 | 26.1 |
| 34 | 20.8 |
| 35 | 29.7 |
| 36 | 15.7 |
| 37 | 13.5 |
| 38 | 34.3 |
| 39 | 20.5 |
| 40 | 21.5 |
| 41 | 21.9 |
| 42 | 23.7 |
| 43 | 21.5 |
| 44 | 34.7 |
| 45 | 24.0 |
| 46 | 18.0 |
| 47 | 27.8 |
| 48 | 37.5 |
| 49 | 37.1 |
| 50 | 23.4 |
| 51 | 9.9 |
| 52 | 26.7 |
| 53 | 22.9 |
| 54 | 27.8 |
| 55 | 29.0 |
| 56 | 36.9 |
| 57 | 15.4 |
| 58 | 10.0 |
| 59 | 29.1 |
| 60 | 18.7 |
| 61 | 33.7 |
| 62 | 26.6 |
| 63 | 19.8 |
| 64 | 36.2 |
| 65 | 29.8 |
| 66 | 25.1 |
| 67 | 18.9 |
| 68 | 22.3 |
| 69 | 33.5 |
| 70 | 14.0 |
| 71 | 26.8 |
| 72 | 15.5 |
| 73 | 19.1 |
| 74 | 19.4 |
| 75 | 20.6 |
| 76 | 20.4 |
| 77 | 24.1 |
| 78 | 23.1 |
| 79 | 26.6 |
| 80 | 47.2 |
| 81 | 33.0 |
| 82 | 31.9 |
| 83 | 34.4 |
| 84 | 35.1 |
| 85 | 20.7 |
| 86 | 36.7 |
| 87 | 27.8 |
| 88 | 9.3 |
| 89 | 11.0 |
| 90 | 19.1 |
| 91 | 34.5 |
| 92 | 13.0 |
| 93 | 12.4 |
| 94 | 20.6 |
| 95 | 21.8 |

Ex Vivo Hair Cell Induction Assay Using 6-Day-Postnatal Mouse Cochlea Explants with Hair Cell Damage

P6, postnatal 6 days, Atoh1-GFP mice, the same mouse strain used for Atoh1 induction assay described before, were used in this assay. The otic capsule was exposed and the cochleae were micro-dissected. The basilar membrane was separated from the organ of Corti and in vitro cultured in serum free medium (culture medium: DMEM/F12+1% N2+2% B27+5 μg/ml ampicillin) at 37° C. under a standard gas atmosphere of humidified air/5% CO₂. Inner ear hair cells were damaged by 1 mM Neomycin treatment for 1.25 h. After the neomycin treatment, explants were cultured in blank culture medium for 7 days before the treatment of selected compounds.

For compound administration, the cochlea explants were treated with 3 to 10 μM compound of the present disclosure, with DMSO as the negative control for 8 days with once compound/medium change. After 8 days treatment, the tested compound was removed. The explants were cultured in blank medium for additional 4 days. The cochlea explant cultures then were fixed with 4% w/v formalin and processed for Myo7a immunofluorescence (Myo7a is a specific marker for sensory hair cell) using the rabbit anti-Myo7a antibody (Protus Biosci #25-6790, 1:250 diluted in PBS containing 3% BSA). Rhodamine labeled Goat-anti-rabbit IgG (Molecular Prob. #R6394, 1:1000 diluted in PBS containing 3% BSA) was used as the secondary antibody to visualize the Myo7a positive cells. The images were collected and analyzed using the EVOS image system (Thermo-Fisher Scientific). It was found that treatment with tested compounds significantly increased the number of Atoh1-GFP and Myo7a positive cells. The hair cell identity of the ectopically formed cells was confirmed by staining the cells with multiple hair cell markers.

The efficacy of hair cells induction in this assay is represented by the responsive length percentage of Atoh1 and Myo7a double positive cells in the damaged whole explants after compound treatment. The responsive length percentage was calculated according to the equation of ((the explant length with Atoh1 and Myo7a double positive cells/the full length of cochlea explant)*100%). Note the value of DMSO control is 0% due to total damage of hair cells, and any responsive length percentage more than 20% is considered as significant hair cell induction. As shown in Table 3, representative compounds of the present disclosure have demonstrated significant hair cell induction.

TABLE 3

| | Responsive | | Responsive |
|---|---|---|---|
| Example No. | length % | Example No. | length % |
| DMSO | 0 | 36 | 28.1 ± 6.3 |
| 8 | 54.0 ± 6.3 | 15 | 50.1 ± 9.9 |
| 48 | 75.8 ± 4.5 | 14 | 45.3 ± 6.7 |
| 47 | 64.4 ± 17.2 | 51 | 48.6 ± 3.0 |
| 59 | 54.1 ± 5.8 | 17 | 52.9 ± 6.8 |
| 10 | 59.1 ± 9.1 | 56 | 26.5 ± 9.4 |
| 40 | 36.9 ± 7.4 | 54 | 35.6 ± 11.8 |
| 27 | 66.0 ± 2.4 | 53 | 34.7 ± 9.8 |
| 55 | 53.1 ± 6.2 | 52 | 28.2 ± 11.9 |
| 67 | 58.2 ± 3.5 | 65 | 43.1 ± 7.6 |
| 66 | 61.5 ± 6.0 | 45 | 51.9 ± 14.9 |
| 42 | 28.8 ± 8.3 | 12 | 39.7 ± 8.2 |
| 11 | 51.7 ± 8.1 | 49 | 47.5 ± 9.2 |

Note:
the responsive length % is a mean ± SD.
SD: standard deviation

What is claimed is:

1. A compound of Formula (I) 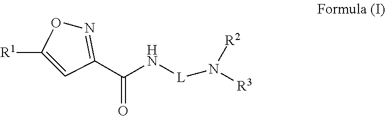
or a pharmaceutically acceptable salt thereof, wherein:
R¹ is selected from phenyl, thienyl, and furanyl, which are each independently optionally substituted by 1-2 F;
L is C₅-C₆ alkylene optionally substituted with 1-4 substituents independently selected from C₁₋₆ alkyl and halogen, wherein optionally a C₁₋₆ alkyl substituent is taken together with the carbon atoms to which it is attached to form a 3-membered cycloalkyl ring;
R² and R³ are taken together with the nitrogen atom to which they are attached to form a 4- to 10-membered heterocyclyl comprising carbon atoms and 1-3 heteroatoms independently selected from N and O, which is optionally substituted with 1-4 R⁴;
each R⁴ is independently selected from C₁₋₆ alkyl, C₃₋₈ cycloalkyl, halogen, (C₀-C₃ alkylene)-CN, C₁₋₆ haloalkyl, C₁-C₆ haloalkoxy, (C₀-C₆ alkylene)-OR⁵, (═O), NH(C═O)R⁵, NH(C═O)OR⁷, NH(C═O)N(R⁵)₂, (C═O)N(R⁷)₂, (C═O)R⁵, (C═O)O(C₁₋₆alkyl), (C═O)O(C₃₋₈ cycloalkyl), S(═O)₂R⁵, S(═O)₂N(R⁷)₂, NHS(═O)₂R⁵, phenyl optionally substituted with 1-3 R⁶ and 5- to 6-membered heteroaryl comprising carbon atoms and 1-3 heteroatoms independently selected from N, O and S optionally substituted with 1-3 R⁶;
each R⁵ is independently selected from H, C₁₋₆ alkyl and C₃₋₈ cycloalkyl;
each R⁶ is independently selected from C₁₋₆ alkyl, C₃₋₈ cycloalkyl, halogen, CN, C₁₋₆ haloalkyl, C₁-C₆ haloalkoxy, OR⁵, N(R⁵)₂, NH(C═O)R⁵, (C═O)N(R⁵)₂, (C═O)R⁵, (C═O)OR⁵, S(═O)₂R⁵ and S(═O)₂N(R⁵)₂; and
each R⁷ is independently selected from H, C₁₋₆ alkyl, C₃₋₈ cycloalkyl optionally substituted with 1-2 OR⁵, (C₀-C₃ alkylene)-CN and (C₀-C₃ alkylene)-OR⁵.

2. The compound or a pharmaceutically acceptable salt thereof according to claim 1, wherein R¹ is selected from phenyl, phenyl substituted with one F, 2-thienyl, 3-thienyl, 2-furanyl and 3-furanyl.

3. The compound or a pharmaceutically acceptable salt thereof according to claim 1, wherein R¹ is

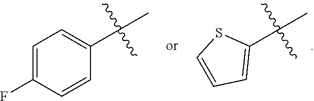 [SMILES: *c1ccc(F)cc1 ; *c1cccs1]

4. The compound or a pharmaceutically acceptable salt thereof according to claim 1, wherein L is C₅ alkylene optionally substituted with 1-4 halogen.

5. The compound or a pharmaceutically acceptable salt thereof according to claim 1, wherein L is C₅ alkylene optionally substituted with two F.

6. The compound or a pharmaceutically acceptable salt thereof according to claim 1, wherein R² and R³ are taken together with the nitrogen atom to which they are attached form a 4- to 10-membered heterocyclyl having the structure selected from: 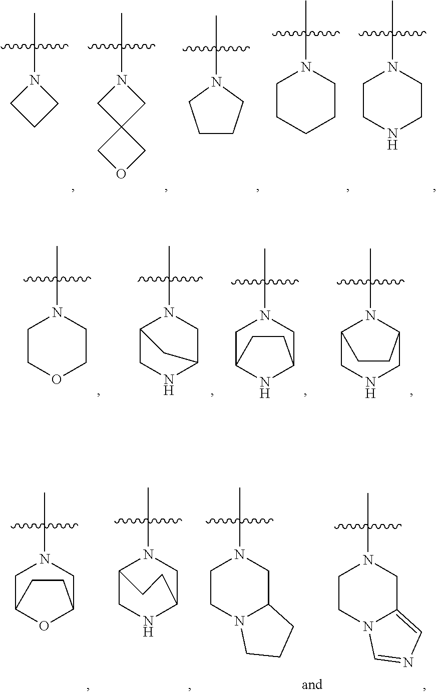
which are each independently optionally substituted with 1-2 R⁴.

7. The compound or a pharmaceutically acceptable salt thereof according to claim 1, wherein R² and R³ are taken together with the nitrogen atom to which they are attached form a 4- to 10-membered heterocyclyl having the structure selected from: 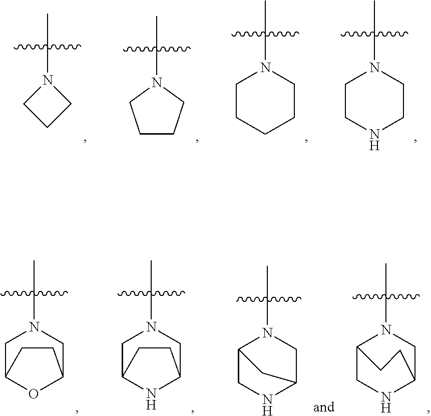
which are each independently optionally substituted with 1-2 R⁴.

8. The compound or a pharmaceutically acceptable salt thereof according to claim 1, wherein each R⁴ is independently selected C₁₋₆ alkyl, halogen, (C₀-C₃ alkylene)-CN, (C₀-C₆ alkylene)-OR⁵, (═O), NH(C═O)R⁵, NH(C═O)OR⁷, NH(C═O)N(R⁵)₂, (C═O)N(R⁷)₂, (C═O)R⁵, (C═O)O(C₁₋₆ alkyl), (C═O)O(C₃₋₈ cycloalkyl), S(═O)₂N(R⁷)₂, NHS(═O)₂R⁵, phenyl optionally substituted with 1-3 R⁶ and 5- to 6-membered heteroaryl comprising carbon atoms and 1-3 heteroatoms independently selected from N, O and S optionally substituted with 1-3 R⁶.

9. The compound or a pharmaceutically acceptable salt thereof according to claim 1, wherein each R⁴ is independently selected from CH₃, CH₂CH(CH₃)₂, F, CN, CH₂—CN, OH, OCH₃, CH₂—OH, (CH₂)₂—OH, NH(C═O)OCH₃, NH(C═O)CH₃, NH(C═O)NHCH₃, (C═O)NH₂, (C═O)NHCH₃, (C═O)NH(cyclopentyl-OH), (C═O)NH(CH₂—CN), (C═O)NH(CH₂CH₂—CN), (C═O)NH(CH₂CH₂—OH), C(═O)CH₃, S(═O)₂NH₂, NHS(═O)₂CH₃, phenyl and imidazolyl.

10. The compound or a pharmaceutically acceptable salt thereof according to claim 1, wherein each R⁴ is independently selected from CH₃, F, (CH₂)₂—OH, (C═O)NH₂, S(═O)₂NH₂, (C═O)NH(CH₂—CN), (C═O)NH(CH₂CH₂—CN), (C═O)NH(cyclopentyl-OH) and NHS(═O)₂CH₃.

11. The compound or a pharmaceutically acceptable salt thereof according to claim 1 selected from:
Example 8: N-(5-(4-methylpiperazin-1-yl)pentyl)-5-(thiophen-2-yl)isoxazole-3-carboxamide;
Example 19: N-(5-(3-(methylsulfonamido)azetidin-1-yl)pentyl)-5-(thiophen-2-yl)isoxazole-3-carboxamide;
Example 27: N-(5-(3-carbamoylazetidin-1-yl)pentyl)-5-(thiophen-2-yl)isoxazole-3-carboxamide;
Example 45: (S)—N-(5-(3-fluoropyrrolidin-1-yl)pentyl)-5-(thiophen-2-yl)isoxazole-3-carboxamide;
Example 51: N-(5-(8-oxa-3-azabicyclo[3.2.1]octan-3-yl)pentyl)-5-(thiophen-2-yl)isoxazole-3-carboxamide;
Example 52: N-(5-(5-methyl-2,5-diazabicyclo[2.2.2]octan-2-yl)pentyl)-5-(thiophen-2-yl)isoxazole-3-carboxamide;
Example 54: N-(5-(4-(2-hydroxyethyl)piperazin-1-yl)pentyl)-5-(thiophen-2-yl)isoxazole-3-carboxamide;
Example 61: N-(5-(3-(methylcarbamoyl)azetidin-1-yl)pentyl)-5-(thiophen-2-yl)isoxazole-3-carboxamide;
Example 65: N-(5-(3-carbamoylazetidin-1-yl)pentyl)-5-(4-fluorophenyl)isoxazole-3-carboxamide;
Example 72: N-(5-(3-sulfamoylazetidin-1-yl)pentyl)-5-(thiophen-2-yl)isoxazole-3-carboxamide;
Example 73: 5-(5-fluorothiophen-2-yl)-N-(5-(4-methylpiperazin-1-yl)pentyl)isoxazole-3-carboxamide;
Example 74: N-(3,3-difluoro-5-(4-methylpiperazin-1-yl)pentyl)-5-(thiophen-2-yl)isoxazole-3-carboxamide;
Example 77: N-(5-(3-((cyanomethyl)carbamoyl)azetidin-1-yl)pentyl)-5-(thiophen-2-yl)isoxazole-3-carboxamide;
Example 83: N-(5-(3-((2-hydroxycyclopentyl)carbamoyl)azetidin-1-yl)pentyl)-5-(thiophen-2-yl) isoxazole-3-carboxamide;
Example 85: 5-(4-fluorophenyl)-N-(5-(3-(methylcarbamoyl)azetidin-1-yl)pentyl)isoxazole-3-carboxamide; and
Example 88: N-(5-(3-((Cyanomethyl)carbamoyl)azetidin-1-yl)pentyl)-5-(4-fluorophenyl)isoxazole-3-carboxamide.

12. A pharmaceutical composition, comprising:
a therapeutically effective amount of a compound of Formula (I) according to claim 1 or a pharmaceutically acceptable salt thereof, and
one or more pharmaceutically acceptable carriers.

13. A pharmaceutical combination, comprising:
a therapeutically effective amount of a compound of Formula (I) according to claim 1 or a pharmaceutically acceptable salt thereof, and
one or more therapeutically active agents.

14. A method of treating hearing loss or balance disorder, comprising administering to a subject in need thereof a therapeutically effective amount of a compound according to claim 1 or a pharmaceutically acceptable salt thereof.
